(19) United States
(12) Patent Application Publication
(10) Pub. No.: US 2004/0167131 A1
(43) Pub. Date: Aug. 26, 2004

(54) Inhibitors of factor Xa and other serine proteases involved in the coagulation cascade

(75) Inventors: Gary Louis Bolton, Ann Arbor, MI (US); Kevin James Filipski, Canton, MI (US); Jeffrey Thomas Kohrt, Ann Arbor, MI (US); Frances La, Gainesville, FL (US); Daniele Marie Leonard, Ann Arbor, MI (US)

Correspondence Address: PFIZER INC, 150 EAST 42ND STREET, 5TH FLOOR - STOP 49, NEW YORK, NY, 10017-5612, US

(73) Assignee: Pfizer Inc.

(21) Appl. No.: 10/662,046

(22) Filed: Sep. 11, 2003

Related U.S. Application Data
(60) Provisional application No. 60/409,891, filed on Sep. 11, 2002.

(51) Int. Cl. A61K 31/537; A61K 31/495

(52) U.S. Cl. 514/237.5; 514/252.12; 514/597; 544/162; 544/399; 564/49; 564/50

(57) ABSTRACT

This invention discloses amino acid derivatives which display inhibitory effects on the serine protease factor Xa. The invention also discloses pharmaceutically acceptable salts of the compounds, pharmaceutically acceptable compositions comprising the compounds or their salts, methods for the preparation of the compounds, and methods of using them as therapeutic agents for treating or preventing disease states in mammals characterized by abnormal thrombosis.

### FIELD OF THE INVENTION

The present invention relates to amino acid derivatives which display inhibitory effects of the serine protease factor Xa. The invention also discloses methods for the preparation of the compounds, pharmaceutically acceptable salts of the compounds, pharmaceutically acceptable compositions comprising the compounds or their salts, and methods of using them as therapeutic agents for treating or preventing disease states in mammals characterized by abnormal thrombosis.

### BACKGROUND OF THE INVENTION

In economically developed countries, cardiovascular disease represents a major cause of mortality. In particular, abnormal coagulation and inappropriate thrombus formation within blood vessels precipitates many acute cardiovascular disease states. While it has long been recognized that a variety of plasma proteins such as fibrinogen, serine proteases, and cellular receptors are involved in hemostasis, it is abnormal regulation that has emerged as important contributing factors to cardiovascular disease.

Thrombin can be considered the key or principal regulatory enzyme in the coagulation cascade; it serves a pluralistic role as both a positive and negative feedback regulator in normal hemostasis. However, in some pathologic conditions, the positive feedback regulation is amplified through catalytic activation of cofactors required for thrombin generation. Such cofactors include factor Xa, a serine protease which occupies a pivotal position in the coagulation cascade. Factor X is the zymogen of factor Xa. Factor X can be activated either the intrinsic or extrinsic pathways of the coagulation system. Initiation of coagulation by either pathway in response to vascular injury activates factor X to factor Xa. Factor Xa and its cofactor, factor Va, combine on a phospholipid membrane to form the “prothombinase” complex, which activates prothrombin to thrombin. Thrombin cleaves fibrinogen to fibrin, activates platelets, and converts factor XIII to XIIIa which is the principal enzyme involved in thrombus generation, growth, and stabilization. Accordingly, the location of the prothrombinase complex at the convergence of both the intrinsic and extrinsic coagulation pathways suggests that inhibition of factor Xa, and hence thrombin generation, may be a viable approach to limiting the procoagulant activity of thrombin.

Evidence exists for the role of factor Xa inhibitors as anticoagulants. Antistasin, a potent inhibitor of blood coagulation factor Xa from the Mexican leech, Haementeria officinalis, displays antithrombotic activity in various models of arterial and venous thrombosis (Lapatto et al., Embo. J, 1997:5151-5161). Other protein or polypeptide factor Xa inhibitors include recombinant tick anticoagulant peptide (rTAP), which is known to accelerate the recombinant tissue plasminogen activator mediated clot lysis and prevent acute reocclusion in the dog, hence indicating factor Xa inhibitors may be useful as an adjunct to thrombolytic therapy (Mellott et al., Fibrinolysis, 1993: 195-202). Furthermore, in a canine coronary artery electrolytic lesion model, rTAP was demonstrated to reduce thrombus mass and time to occlusion in the absence of dramatic hemodynamic or hemostatic changes indicating the primary role for factor Xa in the process of arterial thrombosis (Lynch et al., Thromb. Haemostasis, 1995:640-645; Schaffer et al., Circulation, 1991: 1741- 1748). On the venous side, rTAP was also demonstrated to reduce fibrin deposition in a rabbit model of venous thrombosis while having little affect on systemic hemostatic parameters (Fioravanti et al., Thromb. Res., 1993: 317-324). In addition to these relatively high molecular weight proteins that are not suitable as oral antithrombotic agents, there also exist examples of low molecular weight factor Xa inhibitors. In particular DX9065a, a low molecular weight synthetic factor Xa inhibitor, has also shown antithrombotic potential in various experimental thrombosis rat models. In both arteriovenous shunt and venous stasis models, inhibition of thrombus formation was achieved at doses that had little effect on APTT, indicating that DX9065a is effective in preventing thrombosis and hence has therapeutic antithrombotic potential (Wong et al., Thromb. Res., 1996: 117-126).

Recently, it has been appreciated that factor Xa inhibition may provide sustained antithrombotic protection. Specifically, several animal studies show that inhibition of short term exposure to factor Xa produces a sustained antithrombotic effect. (Leadley, Curr. Top. Med. Chem., 2001: v. 1, 151-159.) Finally, the article by Leadley observes that factor Xa inhibition potentially provides a large therapeutical window between antithrombotic efficacy and bleeding tendency. Consequently, there may exist a range in which factor Xa inhibition is achieved without a concurrent increase in a paitents susceptibility to bleeding.

The majority of factor Xa inhibitors known to date have been summarized in two reviews (Edmunds et al., Annual Reports in Medicinal Chemistry, 1996:51 and Kunitada and Nagahara, Curr. Pharm. Des., 1996:531-542). However, it is readily apparent that there still exists a need for more effective agents that regulate factor Xa proteolytic activity.

### SUMMARY OF THE INVENTION

The present invention provides compounds of Formula I:

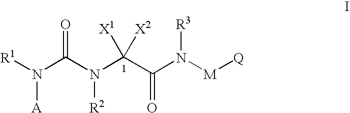

and pharmaceutically acceptable salts thereof, where:

X¹ and X² are hydrogen, alkyl, alkenyl, alkynyl, cycloalkyl, aryl, heteroaryl, aralkyl, cycloalkylalkyl, —(CH₂)ₘ-halogen, —(CH₂)ₘ-heteroaryl, —(CH₂)ₘ—SOR³, —(CH₂)ₘ—OCOR³, —(CH₂)ₘ—OSO₂R³, —(CH₂)ₘ—OSO₂NR⁴R⁵, (CH₂)ₘ—NR⁶COR³, —(CH₂)ₘ—NR⁶SO₂R³, —(CH₂)ₘ—NR³SO₂NR⁴R⁵, —(CH₂)ₘNR⁴R⁵, —(CH₂)ₘOR³, —CN, —NO₂, —CF₍₃₋ₙ₎Hₙ, —(CH₂)ₘ—O(CH₂)ₘR³, —(CH₂)ₘ—O(CH₂)ₘ—OR³, —(CH₂)ₘ—O(CH₂)ₘ—NR⁴R⁵, —(CH₂)ₘR³, —(CH₂)ₘCO₂R³, —(CH₂)ₘCOR³, —(CH₂)ₘCONR⁴R⁵, —(CH₂)ₘNR⁶COR³, —(CH₂)ₘNR⁶CONR⁴R⁵, —(CH₂)ₘSO₂R³, —(CH₂)ₘSO₂NR⁴R⁵,

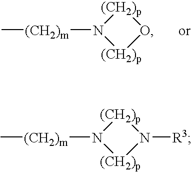

or are joined together to form a substituted or unsubstituted three to eight member ring wherein 0 to 3 atoms of the ring are heteroatoms;

A is aryl, arylcycloalkyl, heteroaryl, heteroarylcycloalkyl, cycloalkyl, or cycloalkenyl;

M is arylene, heteroarylene, cycloalkylene, heterocycloalkylene, cycloalkenylene or heterocycloalkenylene;

Q is —CONR⁴R⁵, aryl, heteroaryl, cycloalkyl, cycloalkenyl, heterocycloalkyl, or heterocycloalkenyl;

R¹ is hydrogen, alkyl, aryl, heteroaryl or alkenyl;

R² is hydrogen, alkyl, aryl, heteroaryl, alkenyl, cycloalkyl, cycloalkylalkyl, aralkyl, heteroaralkyl, heterocycloalkylalkyl, carboxy, —(CH₂)ₘNR⁴R⁵, —(CH₂)ₘOR³, —(CH₂)ₘSR³, —(CH₂)ₘCONR⁴R⁵, or —(CH₂)_{NR}⁶COR³;

R³ is hydrogen, alkyl, aryl, heteroaryl, alkenyl, alkynyl, cycloalkyl, cycloalkylalkyl, aralkyl, or heteroarylalkyl;

R⁶ is hydrogen, alkyl, aryl, heteroaryl, alkenyl, alkynyl, cycloalkyl, cycloalkylalkyl, aralkyl, or heteroarylalkyl;

R⁴ and R⁵ are each independently hydrogen, alkyl, aryl, heteroaryl, alkenyl, alkynyl, cycloalkyl, cycloalkylalkyl, aralkyl, heteroarylalkyl,

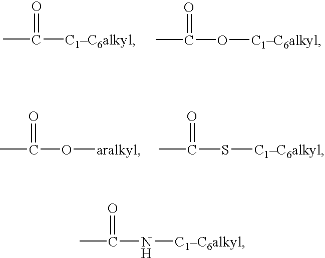

or joined together to form a 3 to 8 member ring;

m is 0 to 8;

n is 0 to 2; and

p is 1 to 3;

with the proviso that when R¹ and R² are H, neither X¹ nor X² is H.

The present invention also provides a compound which is:

1-[3-(4-Chloro-phenyl)-ureido]-cyclopropanecarboxylic acid (3-fluoro-2′-methanesulfonyl-biphenyl-4-yl)-amide;

1-[3-(5-Chloro-pyridin-2-yl)-ureido]-cyclopropanecarboxylic acid (3-fluoro-2′-sulfamoyl-biphenyl-4-yl)-amide;

2-[3-(5-Chloro-pyridin-2-yl)-ureido]-N-(3-fluoro-2′-methanesulfonyl-biphenyl-4-yl)-2-methyl-propionamide;

2-[3-(4-Chloro-phenyl)-ureido]-N-(3-fluoro-2′-sulfamoyl-biphenyl-4-yl)-2-methyl-propionamide;

4-[3-(4-Chloro-phenyl)-ureido]-tetrahydro-thiopyran-4-carboxylic acid (3-fluoro-2′-sulfamoyl-biphenyl-4-yl)-amide;

1-[3-(4-Chloro-phenyl)-ureido]-cyclopropanecarboxylic acid (3-fluoro-2′-sulfamoyl-biphenyl-4-yl)-amide;

1-[3-(5-Chloro-pyridin-2-yl)-ureido]-cyclopropanecarboxylic acid (3-fluoro-2′-methanesulfonyl-biphenyl-4-yl)-amide;

4-[3-(4-Chloro-phenyl)-ureido]-tetrahydro-pyran-4-carboxylic acid (3-fluoro-2′-sulfamoyl-biphenyl-4-yl)-amide;

1-[3-(4-Chloro-phenyl)-ureido]-cyclopentanecarboxylic acid (2′-methanesulfonyl-biphenyl-4-yl)-amide;

1-[3-(4-Chloro-phenyl)-ureido]-cyclohexanecarboxylic acid (2′-methanesulfonyl-biphenyl-4-yl)-amide;

2-[3-(4-Chloro-phenyl)-1-methyl-ureido]-N-(3-fluoro-2′-methanesulfonyl-biphenyl-4-yl)-acetamide;

2-[3-(4-Chloro-phenyl)-1,3-dimethyl-ureido]-N-(3-fluoro-2′-methanesulfonyl-biphenyl-4-yl)-acetamide;

2-[3-(4-Chloro-phenyl)-ureido]-3-hydroxy-2-hydroxymethyl-N-(2′-sulfamoyl-biphenyl-4-yl)-propionamide;

1-[3-(4-Chloro-phenyl)-ureido]-cyclopropanecarboxylic acid (2′-methanesulfonyl-biphenyl-4-yl)-amide;

2-[3-(4-Chloro-phenyl)-ureido]-N-(3-fluoro-2′-methanesulfonyl-biphenyl-4-yl)-2-methyl-propionamide;

2-[3-(4-Chloro-phenyl)-ureido]-N-(3-fluoro-2′-methanesulfonyl-biphenyl4-yl)-acetamide;

1-[3-(4-Chloro-phenyl)-ureido]-cyclopent-3-enecarboxylic acid (3-fluoro-2′-sulfamoyl-biphenyl-4-yl)-amide; and

2-[3-(4-Chloro-phenyl)-3-methyl-ureido]-N-(3-fluoro-2′-methanesulfonyl-biphenyl-4-yl)-acetamide;

(1S,2S)-1-[3-(4-Chloro-phenyl)-ureido]-2-hydroxymethyl-cyclopropanecarboxylic acid (3-fluoro-2′-methanesulfonyl-biphenyl-4-yl)-amide;

(1R,2S)-1-[3-(4-Chloro-phenyl)-ureido]-2-hydroxymethyl-cyclopropanecarboxylic acid (3-fluoro-2′-methanesulfonyl-biphenyl-4-yl)-amide;

(1R, 2S)-1-[3-(4-Chloro-phenyl)-ureido]-2-hydroxymethyl-cyclopropanecarboxylic acid [2-fluoro-4-(2-oxo-piperidin-1-yl)-phenyl]-amide;

(1S, 2S)-1-[3-(4-Chloro-phenyl)-ureido]-2-hydroxymethyl-cyclopropanecarboxylic acid [2-fluoro-4-(2-oxo-piperidin-1-yl)-phenyl]-amide;

2-[3-(4-Chloro-phenyl)-ureido]-N-[2-fluoro-4-(2-oxo-piperidin-1-yl)-phenyl]-2-methyl-propionamide;

2-[3-(5-Chloro-pyridin-2-yl)-ureido]-2-methyl-N-[4-(2-oxo-piperidin-1-yl)-phenyl]-propionamide;

2-[3-(4-Chloro-phenyl)-ureido]-2-methyl-N-[4-(2-oxo-piperidin-1-yl)-phenyl]-propionamide;

2-[3-(5-Chloro-pyridin-2-yl)-ureido]-N-[2-fluoro-4-(2-oxo-piperidin-1-yl)-phenyl]-2-methyl-propion amide;

N-[2-Fluoro-4-(2-oxo-piperidin-1-yl)-phenyl]-2-[3-(4-fluoro-phenyl)-ureido]-2-methyl-propionamide;

1-[3-(4-Chloro-phenyl)-ureido]-cyclopropanecarboxylic acid [2-fluoro-4-(2-oxo-piperidin-1-yl)-phenyl]-amide;

1-[3-(5-Chloro-pyridin-2-yl)-ureido]-cyclopropanecarboxylic acid [4-(2-oxo-piperidin-1-yl)-phenyl]-amide;

1-[3-(4-Chloro-phenyl)-ureido]-cyclopropanecarboxylic acid [4-(2-oxo-piperidin-1-yl)-phenyl]-amide;

1-[3-(5-Chloro-pyridin-2-yl)-ureido]-cyclopropanecarboxylic acid [2-fluoro-4-(2-oxo-piperidin-1-yl)-phenyl]-amide;

1-[3-(4-Fluoro-phenyl)-ureido]-cyclopropanecarboxylic acid [2-fluoro-4-(2-oxo-piperidin-1-yl)-phenyl)-amide;

1-[3-(4-Chloro-phenyl)-ureido]-cyclohexanecarboxylic acid [2-fluoro-4-(2-oxo-piperidin-1-yl)-phenyl]-amide;

1-[3-(5-Chloro-pyridin-2-yl)-ureido]-cyclohexanecarboxylic acid [4-(2-oxo-piperidin-1-yl)-phenyl]-amide;

1-[3-(4-Chloro-phenyl)-ureido]-cyclohexanecarboxylic acid [4-(2-oxo-pipelidin-1-yl)-phenyl]-amide;

1-[3-(5-Chloro-pyridin-2-yl)-ureido]-cyclohexanecarboxylic acid [2-fluoro-4-(2-oxo-piperidin-1-yl)-phenyl]-amide;

1-[3-(4-Fluoro-phenyl)-ureido]-cyclohexanecarboxylic acid [2-fluoro-4-(2-oxo-piperidin-1-yl)-phenyl]-amide;

2-[3-(4-Chloro-phenyl)-ureido]-N-[2-fluoro-4-(2-oxo-piperidin-1-yl)-phenyl]-3-hydroxy-2-hydroxymethyl-propionamide;

2-[3-(5-Chloro-pyridin-2-yl)-ureido]-3-hydroxy-2-hydroxymethyl-N-[4-(2-oxo-piperidin-1-yl)-phenyl]-propionamide;

2-[3-(4-Chloro-phenyl)-ureido]-3-hydroxy-2-hydroxymethyl-N-[4-(2-oxo-piperidin-1-yl)-phenyl]-propionamide;

2-[3-(5-Chloro-pyridin-2-yl)-ureido]-N-[2-fluoro-4-(2-oxo-piperidin-1-yl)-phenyl]-3-hydroxy-2-hydroxymethyl-propionamide;

N-[2-Fluoro-4-(2-oxo-piperidin-1-yl)-phenyl]-2-[3-(4-fluoro-phenyl)-ureido]-3-hydroxy-2-hydroxymethyl-propionamide;

2-[3-(4-Chloro-phenyl)-ureido]-N-[2-fluoro-4-(2-oxo-piperidin-1-yl)-phenyl]-acetamide;

2-[3-(5-Chloro-pyridin-2-yl)-ureido]-N-[2-fluoro-4-(2-oxo-piperidin-1-yl)-phenyl]-acetamide;

2-[3-(5-Chloro-pyridin-2-yl)-ureido]-N-[4-(2-oxo-piperidin-1-yl)-phenyl]-acetamide;

2-[3-(4-Chloro-phenyl)-ureido]-N-[4-(2-oxo-piperidin-1-yl)-phenyl]-acetamide;

1-[3-(4-Chloro-phenyl)-ureido]-cyclopropanecarboxylic acid [5-(2-methanesulfonyl-phenyl)-pyridin-2-yl]-amide;

1-[3-(4-Chloro-phenyl)-ureido]-cyclopropanecarboxylic acid [5-(2-sulfamoyl-phenyl)-pyridin-2-yl]-amide;

1-[3-(5-Chloro-pyridin-2-yl)-ureido]-cyclopropanecarboxylic acid [5-(2-methanesulfonyl-phenyl)-pyridin-2-yl]-amide;

1-[3-(5-Chloro-pyridin-2-yl)-ureido]-cyclopropanecarboxylic acid [5-(2-sulfamoyl-phenyl)-pyridin-2-yl]-amide;

1-[3-(4-Chloro-phenyl)-ureido]-cyclopropanecarboxylic acid (2′-methanesulfonyl-3-trifluoromethyl-biphenyl-4-yl)-amide;

1-[3-(4-Chloro-phenyl)-ureido]-cyclopropanecarboxylic acid (2′-sulfamoyl-3-trifluoromethyl-biphenyl-4-yl)-amide;

1-[3-(5-Chloro-pyridin-2-yl)-ureido]-cyclopropanecarboxylic acid (2′-methanesulfonyl-3-trifluoromethyl-biphenyl-4-yl)-amide;

1-[3-(5-Chloro-pyridin-2-yl)-ureido]-cyclopropanecarboxylic acid (2′-sulfamoyl-3-trifluoromethyl-biphenyl-4-yl)-amide;

1-[3-(4-Chloro-phenyl)-ureido]-cyclopropanecarboxylic acid (2′-methanesulfonyl-3-methyl-biphenyl-4-yl)-amide;

1-[3-(4-Chloro-phenyl)-ureido]-cyclopropanecarboxylic acid (3-methyl-2′-sulfamoyl-biphenyl-4-yl)-amide;

1-[3-(5-Chloro-pyridin-2-yl)-ureido]-cyclopropanecarboxylic acid (2′-methanesulfonyl-3-methyl-biphenyl-4-yl)-amide;

1-[3-(5-Chloro-pyridin-2-yl)-ureido]-cyclopropanecarboxylic acid (3-methyl-2′-sulfamoyl-biphenyl-4-yl)-amide;

2-[3-(5-Chloro-pyridin-2-yl)-1-methyl-ureido]-N-(2′-methanesulfonyl-biphenyl-4-yl)-acetamide;

2-[3-(4-Chloro-phenyl)-1-methyl-ureido]-N-(2′-sulfamoyl-biphenyl-4-yl)-acetamide;

2-[3-(5-Chloro-pyridin-2-yl)-1-methyl-ureido]-N-(3-fluoro-2′-sulfamoyl-biphenyl-4-yl)-acetamide;

2-[3-(4-Chloro-phenyl)-1-methyl-ureido]-N-(3-fluoro-2′-sulfamoyl-biphenyl-4-yl)-acetamide;

2-[3-(5-Chloro-pyridin-2-yl)-1-methyl-ureido]-N-(3-fluoro-2′-methanesulfonyl-biphenyl-4-yl)-acetamide;

2-[3-(4-Chloro-phenyl)-1-methyl-ureido]-N-(2′-methanesulfonyl-biphenyl-4-yl)-acetamide;

1-[3-(4-Chloro-phenyl)-ureido]-2-hydroxymethyl-cyclopropanecarboxylic acid (2′-methanesulfonyl-biphenyl-4-yl)-amide;

1-[3-(4-Chloro-phenyl)-ureido]-2-hydroxymethyl-cyclopropanecarboxylic acid (2′-sulfamoyl-biphenyl-4-yl)-amide;

1-[3-(4-Chloro-phenyl)-ureido]-2-hydroxymethyl-cyclopropanecarboxylic acid (3-fluoro-2′-methanesulfonyl-biphenyl-4-yl)-amide;

1-[3-(4-Chloro-phenyl)-ureido]-2-hydroxymethyl-cyclopropanecarboxylic acid (3-fluoro-2′-sulfamoyl-biphenyl-4-yl)-amide;

3-[3-(4-Chloro-phenyl)-ureido]-pyrrolidine-3-carboxylic acid (3-fluoro-2′-methanesulfonyl-biphenyl-4-yl)-amide;

3-[3-(4-Chloro-phenyl)-ureido]-pyrrolidine-3-carboxylic acid (3-fluoro-2′-sulfamoyl-biphenyl-4-yl)-amide;

1-[3-(4-Chloro-phenyl)-ureido]-3-hydroxymethyl-cyclobutanecarboxylic acid (3-fluoro-2′-methanesulfonyl-biphenyl-4-yl)-amide;

1-[3-(4-Chloro-phenyl)-ureido]-3-hydroxymethyl-cyclobutanecarboxylic acid (3-fluoro-2′-sulfamoyl-biphenyl-4-yl)-amide;

1-[3-(4-Chloro-phenyl)-ureido]-2-methoxymethyl-cyclopropanecarboxylic acid (3-fluoro-2′-methanesulfonyl-biphenyl-4-yl)-amide;

1-[3-(4-Chloro-phenyl)-ureido]-2-methoxymethyl-cyclopropanecarboxylic acid (3-fluoro-2′-sulfamoyl-biphenyl-4-yl)-amide;

2-Aminomethyl-1-[3-(4-chloro-phenyl)-ureido]-cyclopropanecarboxylic acid (3-fluoro-2′-methanesulfonyl-biphenyl-4-yl)-amide;

2-Aminomethyl-1-[3-(4-chloro-phenyl)-ureido]-cyclopropanecarboxylic acid (3-fluoro-2′-sulfamoyl-biphenyl-4-yl)-amide;

2-[3-(4-Chloro-phenyl)-ureido]-2-(3-fluoro-2′-methanesulfonyl-biphenyl-4-ylcarbamoyl)-cyclopropanecarboxylic acid;

2-[3-(4-Chloro-phenyl)-ureido]-2-(3-fluoro-2′-sulfamoyl-biphenyl-4-ylcarbamoyl)-cyclopropanecarboxylic acid;

3-[3-(4-Chloro-phenyl)-ureido]-1-methyl-pyrrolidine-3-carboxylic acid (3-fluoro-2′-methanesulfonyl-biphenyl-4-yl)-amide;

3-[3-(4-Chloro-phenyl)-ureido]-1-methyl-pyrrolidine-3-carboxylic acid (3-fluoro-2′-sulfamoyl-biphenyl-4-yl)-amide;

1-Acetyl-3-[3-(4-chloro-phenyl)-ureido]-pyrrolidine-3-carboxylic acid (3-fluoro-2′-methanesulfonyl-biphenyl-4-yl)-amide;

1-Acetyl-3-[3-(4-chloro-phenyl)-ureido]-pyrrolidine-3-carboxylic acid (3-fluoro-2′-sulfamoyl-biphenyl-4-yl)-amide;

1-[3-(4-Chloro-phenyl)-ureido]-3-methoxymethyl-cyclobutanecarboxylic acid (3-fluoro-2′-methanesulfonyl-biphenyl-4-yl)-amide;

1-[3-(4-Chloro-phenyl)-ureido]-3-methoxymethyl-cyclobutanecarboxylic acid (3-fluoro-2′-sulfamoyl-biphenyl-4-yl)-amide;

3-Aminomethyl-1-[3-(4-chloro-phenyl)-ureido]-cyclobutanecarboxylic acid (3-fluoro-2′-methanesulfonyl-biphenyl-4-yl)-amide;

3-Aminomethyl-1-[3-(4-chloro-phenyl)-ureido]-cyclobutanecarboxylic acid (3-fluoro-2′-sulfamoyl-biphenyl-4-yl)-amide;

3-[3-(4-Chloro-phenyl)-ureido]-3-(3-fluoro-2′-methanesulfonyl-biphenyl-4-ylcarbamoyl)-cyclobutanecarboxylic acid;

3-[3-(4-Chloro-phenyl)-ureido]-3-(3-fluoro-2′-sulfamoyl-biphenyl-4-ylcarbamoyl)-cyclobutanecarboxylic acid;

4-[3-(4-Chloro-phenyl)-ureido]-piperidine-4-carboxylic acid (3-fluoro-2′-methanesulfonyl-biphenyl-4-yl)-amide;

4-[3-(4-Chloro-phenyl)-ureido]-piperidine-4-carboxylic acid (3-fluoro-2′-sulfamoyl-biphenyl-4-yl)-amide;

4-[3-(4-Chloro-phenyl)-ureido]-1-methyl-piperidine-4-carboxylic acid (3-fluoro-2′-methanesulfonyl-biphenyl-4-yl)-amide;

4-[3-(4-Chloro-phenyl)-ureido]-1-methyl-piperidine-4-carboxylic acid (3-fluoro-2′-sulfamoyl-biphenyl-4-yl)-amide;

1-Acetyl-4-[3-(4-chloro-phenyl)-ureido]-piperidine-4-carboxylic acid (3-fluoro-2′-methanesulfonyl-biphenyl-4-yl)-amide;

1-Acetyl-4-[3-(4-chloro-phenyl)-ureido]-piperidine-4-carboxylic acid (3-fluoro-2′-sulfamoyl-biphenyl-4-yl)-amide;

1-[3-(4-Chloro-phenyl)-ureido]-3,4-dihydroxy-cyclopentanecarboxylic acid (3-fluoro-2′-methanesulfonyl-biphenyl-4-yl)-amide;

1-[3-(4-Chloro-phenyl)-ureido]-3,4-dihydroxy-cyclopentanecarboxylic acid (3-fluoro-2′-sulfamoyl-biphenyl-4-yl)-amide;

3-[3-(4-Chloro-phenyl)-ureido]-tetrahydro-furan-3-carboxylic acid (3-fluoro-2′-methanesulfonyl-biphenyl-4-yl)-amide;

3-[3-(4-Chloro-phenyl)-ureido]-tetrahydro-furan-3-carboxylic acid (3-fluoro-2′-sulfamoyl-biphenyl-4-yl)-amide;

3-[3-(4-Chloro-phenyl)-ureido]-tetrahydro-thiophene-3-carboxylic acid (3-fluoro-2′-methanesulfonyl-biphenyl-4-yl)-amide;

3-[3-(4-Chloro-phenyl)-ureido]-1-methyl-pyrrolidine-3-carboxylic acid [2-fluoro-4-(2-oxo-piperidin-1-yl)-phenyl]-amide;

1-Acetyl-3-[3-(4-chloro-phenyl)-ureido]-pyrrolidine-3-carboxylic acid [2-fluoro-4-(2-oxo-piperidin-1-yl)-phenyl]-amide;

1-Acetyl-3-[3-(4-chloro-phenyl)-ureido]-azetidine-3-carboxylic acid (3-fluoro-2′-methanesulfonyl-biphenyl-4-yl)-amide;

1-Acetyl-3-[3-(4-chloro-phenyl)-ureido]-azetidine-3-carboxylic acid (3-fluoro-2′-sulfamoyl-biphenyl-4-yl)-amide;

1-[3-(4-Chloro-phenyl)-1-methyl-ureido]-2-hydroxymethyl-cyclopropanecarboxylic acid [2-fluoro-4-(2-oxo-piperidin-1-yl)-phenyl]-amide;

1-[3-(4-Chloro-phenyl)-1-methyl-ureido]-2-hydroxymethyl-cyclopropanecarboxylic acid (3-fluoro-2′-sulfamoyl-biphenyl-4-yl)-amide;

1-[3-(4-Chloro-phenyl)-1-methyl-ureido]-2-hydroxymethyl-cyclopropanecarboxylic acid (3-fluoro-2′-methanesulfonyl-biphenyl-4-yl)-amide;

3-[3-(4-Chloro-phenyl)-ureido]-tetrahydro-thiophene-3-carboxylic acid (3-fluoro-2′-sulfamoyl-biphenyl-4-yl)-amide;

3-[3-(4-Chloro-phenyl)-ureido]-1-methyl-azetidine-3-carboxylic acid (3-fluoro-2′-methanesulfonyl-biphenyl-4-yl)-amide;

3-[3-(4-Chloro-phenyl)-ureido]-1-methyl-azetidine-3-carboxylic acid (3-fluoro-2′-sulfamoyl-biphenyl-4-yl)-amide;

1-[3-(4-Chloro-phenyl)-ureido]-2-hydroxymethyl-cyclopropanecarboxylic acid [2-fluoro-4-(2-oxo-piperidin-1-yl)-phenyl]-amide;

1-[3-(4-Chloro-phenyl)-ureido]-2-methoxymethyl-cyclopropanecarboxylic acid [2-fluoro-4-(2-oxo-piperidin-1-yl)-phenyl]-amide;

3-Amino-2-aminomethyl-2-[3-(4-chloro-phenyl)-ureido]-N-(3-fluoro-2′-sulfamoyl-biphenyl-4-yl)-propionamide;

3-Amino-2-aminomethyl-2-[3-(4-chloro-phenyl)-ureido]-N-(3-fluoro-2′-methanesulfonyl-biphenyl-4-yl)-propionamide;

2-[3-(4-Chloro-phenyl)-ureido]-3-ethylamino-2-ethylaminomethyl-N-(3-fluoro-2′-methanesulfonyl-biphenyl-4-yl)-propionamide;

2-[3-(4-Chloro-phenyl)-1-cyclopropylmethyl-ureido]-N-(3-fluoro-2′-sulfamoyl-biphenyl-4-yl)-acetamide;

2-[3-(4-Chloro-phenyl)-1-cyclopropylmethyl-ureido]-N-[2-fluoro-4-(2-oxo-piperidin-1-yl)-phenyl]-acetaminde;

2-[3-(4-Chloro-phenyl)-1-cyclopropylmethyl-ureido]-N-[2-fluoro-4-(2-oxo-2H-pyridin-1-yl)-phenyl]-acetamide;

2-[3-(5-Chloro-pyridin-2-yl)-1-cyclopropylmethyl-ureido]-N-(3-fluoro-2′-methanesulfonyl-biphenyl-4-yl)-acetamide;

2-[3-(4-Chloro-phenyl)-1-cyclopropyl-ureido]-N-(3-fluoro-2′-methanesulfonyl-biphenyl-4-yl)-acetamide;

2-[3-(5-Chloro-pyridin-2-yl)-1-cyclopropylmethyl-ureido]-N-(3-fluoro-2′-sulfamoyl-biphenyl-4-yl)-acetamide;

2-[3-(5-Chloro-pyridin-2-yl)-1-cyclopropylmethyl-ureido]-N-[2-fluoro-4-(2-oxo-piperidin-1-yl)-phenyl]-acetamide;

2-[3-(5-Chloro-pyridin-2-yl)-1-cyclopropylmethyl-ureido]-N-[2-fluoro-4-(2-oxo-2H-pyridin-1-yl)-phenyl]-acetamide;

2-[3-(4-Chloro-phenyl)-1-isopropyl-ureido]-N-(3-fluoro-2′-methanesulfonyl-biphenyl-4-yl)-acetamide;

2-[3-(4-Chloro-phenyl)-1-cyclopentyl-ureido]-N-(3-fluoro-2′-methanesulfonyl-biphenyl-4-yl)-acetamide;

2-[3-(4-Chloro-phenyl)-1-cyclopentylmethyl-ureido]-N-(3-fluoro-2′-methanesulfonyl-biphenyl-4-yl)-acetamide;

2-[3-(4-Chloro-phenyl)-1-(2-cyclopropyl-ethyl)-ureido]-N-(3-fluoro-2′-methanesulfonyl-biphenyl-4-yl)-acetamide;

2-[3-(4-Chloro-phenyl)-1-phenyl-ureido]-N-(3-fluoro-2′-methanesulfonyl-biphenyl-4-yl)-acetamide;

2-[3-(4-Chloro-phenyl)-1-thiophen-3-ylmethyl-ureido]-N-(3-fluoro-2′-methanesulfonyl-biphenyl-4-yl)-acetamide;

2-[3-(4-Chloro-phenyl)-1-pyridin-3-ylmethyl-ureido]-N-(3-fluoro-2′-methanesulfonyl-biphenyl-4-yl)-acetamide;

2-[3-(4-Chloro-phenyl)-1-cyclohexylmethyl-ureido]-N-(3-fluoro-2′-methanesulfonyl-biphenyl-4-yl)-acetamide;

2-[3-(4-Chloro-phenyl)-1-(2-cyclopentyl-ethyl)-ureido]-N-(3-fluoro-2′-methanesulfonyl-biphenyl-4-yl)-acetamide;

2-[3-(4-Chloro-phenyl)-1-thiophen-2-ylmethyl-ureido]-N-(3-fluoro-2′-methanesulfonyl-biphenyl-4-yl)-acetamide;

2-[3-(4-Chloro-phenyl)-1-pyridin-2-ylmethyl-ureido]-N-(3-fluoro-2′-methanesulfonyl-biphenyl-4-yl)-acetamide;

2-[3-(4-Chloro-phenyl)-1-pyridin-4-ylmethyl-ureido]-N-(3-fluoro-2′-methanesulfonyl-biphenyl-4-yl)-acetamide;

2-[3-(4-Chloro-phenyl)-1-(2-ethoxy-ethyl)-ureido]-N-(3-fluoro-2′-methanesulfonyl-biphenyl-4-yl)-acetamide;

2-[3-(4-Chloro-phenyl)-1-(2-methylsulfanyl-ethyl)-ureido]-N-(3-fluoro-2′-methanesulfonyl-biphenyl-4-yl)-acetamide;

{3-(4-Chloro-phenyl)-1-[(3-fluoro-2′-methanesulfonyl-biphenyl-4-ylcarbamoyl)-methyl]-ureido}-acetic acid;

2-[3-(4-Chloro-phenyl)-1-(2-morpholin-4-yl-ethyl)-ureido]-N-(3-fluoro-2′-methanesulfonyl-biphenyl-4-yl)-acetamide;

2-[3-(4-Chloro-phenyl)-1-(2-thiomorpholin-4-yl-ethyl)-ureido]-N-(3-fluoro-2′-methanesulfonyl-biphenyl-4-yl)-acetamide;

2-[3-(4-Chloro-phenyl)-1-phenethyl-ureido]-N-(3-fluoro-2′-methanesulfonyl-biphenyl-4-yl)-acetamide;

2-[3-(4-Chloro-phenyl)-1-(2-methylsulfanyl-ethyl)-ureido]-N-(3-fluoro-2′-methanesulfonyl-biphenyl-4-yl)-acetamide;

2-[3-(4-Chloro-phenyl)-1-methylcarbamoylmethyl-ureido]-N-(3-fluoro-2′-methanesulfonyl-biphenyl-4-yl)-acetamide;

2-{3-(4-Chloro-phenyl)-1-[2-(4-methyl-piperazin-1-yl)-ethyl]-ureido }-N-(3-fluoro-2′-methanesulfonyl-biphenyl-4-yl)-acetamide;

2-[1-(2-Acetylamino-ethyl)-3-(4-chloro-phenyl)-ureido]-N-(3-fluoro-2′-methanesulfonyl-biphenyl-4-yl)-acetamide;

2-[3-(4-Chloro-phenyl)-1-(2,2-dimethyl-propyl)-ureido]-N-(3-fluoro-2′-methanesulfonyl-biphenyl-4-yl)-acetamide;

3-[3-(4-Chloro-phenyl)-ureido]-3-(3-fluoro-2′-methanesulfonyl-biphenyl-4-ylcarbamoyl)-pyrrolidine-1-carboxylic acid benzyl ester;

2-[3-(4-Chloro-phenyl)-1-(2,2-dimethyl-propyl)-ureido]-N-[2-fluoro-4-(2-oxo-piperidin-1-yl)-phenyl]-acetamide;

2-[3-(4-Chloro-phenyl)-1-cyclobutylmethyl-ureido]-N-(3-fluoro-2′-methanesulfonyl-biphenyl-4-yl)-acetamide;

2-[3-(4-Chloro-phenyl)-1-cyclopropylmethyl-ureido]-N-(3-fluoro-2′-methanesulfonyl-biphenyl-4-yl)-acetamide;

2-[3-(4-Chloro-phenyl)-1-(2-methoxy-ethyl)-ureido]-N-(3-fluoro-2′-methanesulfonyl-biphenyl-4-yl)-acetamide;

2-[3-(4-Chloro-phenyl)-1-isobutyl-ureido]-N-(3-fluoro-2′-methanesulfonyl-biphenyl-4-yl)-acetamide;

2-[3-(4-Chloro-phenyl)-1-(2-dimethylamino-ethyl)-ureido]-N-(3-fluoro-2′-methanesulfonyl-biphenyl-4-yl)-acetamide;

2-[1-Benzyl-3-(4-chloro-phenyl)-ureido]-N-(3-fluoro-2′-methanesulfonyl-biphenyl-4-yl)-acetamide;

2-[3-(4-Chloro-phenyl)-1-(4-methoxy-benzyl) ureido]- N-(3-fluoro-2′-methanesulfonyl-biphenyl-4-yl)-acetamide;

(1R,2S)-(1-[3-(4-Chloro-phenyl)-ureido]-2-hydroxymethyl-cyclopropanecarboxylic acid [2-fluoro-4-(2-oxo-2H-pyridin-1-yl)-phenyl]-amide;

(1S,2S)-1-[3-(4-Chloro-phenyl)-ureido]-2-hydroxymethyl-cyclopropanecarboxylic acid [2-fluoro-4-(2-oxo-2H-pyridin-1-yl)-phenyl]-amide;

1-[3-(4-Chloro-phenyl)-ureido]-cyclopropanecarboxylic acid [2-fluoro-4-(2-oxo-2H-pyridin-1-yl)-phenyl]-amide;

1-[3-(4-Chloro-phenyl)-ureido]-cyclopropanecarboxylic acid [2-fluoro-4-(5-methyl-pyrazol-1-yl)-phenyl]-amide;

(1R,2S)-1-[3-(4-Chloro-phenyl)-ureido]-2-hydroxymethyl-cyclopropanecarboxylic acid [2-fluoro-4-(5-methyl-pyrazol-1-yl)-phenyl]-amide;

(1S,2S)-1-[3-(4-Chloro-phenyl)-ureido]-2-hydroxymethyl-cyclopropanecarboxylic acid [2-fluoro-4-(5-methyl-pyrazol-1-yl)-phenyl]-amide;

2-[3-(4-Chloro-phenyl)-1-cyclopropylmethyl-ureido]-N-[2-fluoro-4-(5-methyl-pyrazol-1-yl)-phenyl]-acetamide;

1-[3-(4-Chloro-phenyl)-ureido]-cyclopropanecarboxylic acid [4-(3,5-dimethyl-pyrazol-1-yl)-2-fluoro-phenyl]-amide;

2-[3-(4-Chloro-phenyl)-1-cyclopropylmethyl-ureido]-N-[4-(3,5-dimethyl-pyrazol-1-yl)-2-fluoro-phenyl]-acetamide;

(1R,2S)-1-[3-(4-Chloro-phenyl)-ureido]-2-hydroxymethyl-cyclopropanecarboxylic acid (2-fluoro-4-pyrazol-1-yl-phenyl)-amide;

(1S,2S)-1-[3-(4-Chloro-phenyl)-ureido]-2-hydroxymethyl-cyclopropanecarboxylic acid (2-fluoro-4-pyrazol-1-yl-phenyl)-amide;

(1R,2S)-1-[3-(4-Chloro-phenyl)-ureido]-2-hydroxymethyl-cyclopropanecarboxylic acid [2-fluoro-4-(3-methyl-pyrazol-1-yl)-phenyl]-amide;

(1S,2S)-1-[3-(4-Chloro-phenyl)-ureido]-2-hydroxymethyl-cyclopropanecarboxylic acid [2-fluoro-4-(3-methyl-pyrazol-1-yl)-phenyl]-amide;

(1R,2S)-1-[3-(4-Chloro-phenyl)-ureido]-2-hydroxymethyl-cyclopropanecarboxylic acid [2-fluoro-4-(2-methyl-imidazol-1-yl)-phenyl]-amide;

(1S,2S)-1-[3-(4-Chloro-phenyl)-ureido]-2-hydroxymethyl-cyclopropanecarboxylic acid [2-fluoro-4-(2-methyl-imidazol-1-yl)-phenyl]-amide;

(1R,2S)-1-[3-(4-Chloro-phenyl)-ureido]-2-hydroxymethyl-cyclopropanecarboxylic acid [4-(2,5-dihydro-pyrrole-1-carbonyl)-2-fluoro-phenyl]-amide;

(1S,2S)-1-[3-(4-Chloro-phenyl)-ureido]-2-hydroxymethyl-cyclopropanecarboxylic acid [4-(2,5-dihydro-pyrrole-1-carbonyl)-2-fluoro-phenyl]-amide;

(1R,2S)-1-[3-(4-Chloro-phenyl)-ureido]-2-hydroxymethyl-cyclopropanecarboxylic acid [2-fluoro-4-(pyrrolidine-1-carbonyl)-phenyl]-amide;

(1S,2S)-1-[3-(4-Chloro-phenyl)-ureido]-2-hydroxymethyl-cyclopropanecarboxylic acid [2-fluoro-4-(pyrrolidine-1-carbonyl)-phenyl]-amide;

(1R,2S)-2-(Acetylamino-methyl)-1-[3-(4-chloro-phenyl)-ureido]-cyclopropanecarboxylic acid (3-fluoro-2′-methanesulfonyl-biphenyl-4-yl)-amide;

(1S,2S)-2-(Acetylamino-methyl)-1-[3-(4-chloro-phenyl)-ureido]-cyclopropanecarboxylic acid (3-fluoro-2′-methanesulfonyl-biphenyl-4-yl)-amide;

or a pharmaceutically acceptable salt thereof.

In yet another embodiment of the present invention, a method for preparing amino acid derivatives described by Formula I is provided. The method for preparing amino acid derivatives described by Formula I, where P¹ is a protecting group, Y¹ is a halogen and X¹, X², A, M, and Q are as defined above, includes

(a) contacting an amino acid having Formula III with a reagent capable of forming a protecting group on the amino group of the amino acid to form a compound with Formula IV

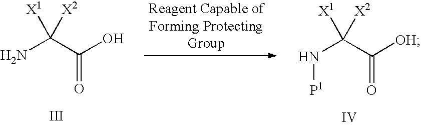

(b) activating of the carboxylic acid of Formula IV and contacting it with an amino compound of the formula H₂N-M-Y¹ to form a compound of Formula V

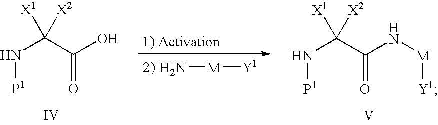

(c) coupling the compound of Formula V with a compound having Q to form a compound of Formula VI

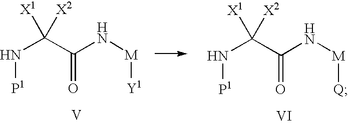

(d) removing the amino protecting group of the compound of Formula VI and contacting the resulting free amine with an isocyanate having A to form a compound of Formula I

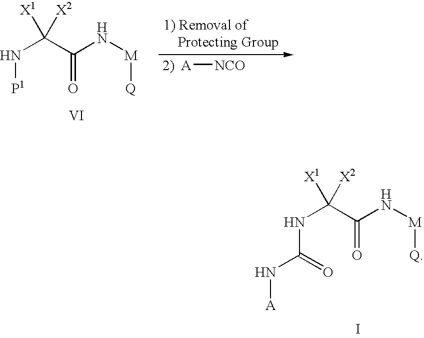

In yet another embodiment of the present invention, a method for preparing amino acid derivatives described by Formula I is provided. The method for preparing amino acid derivatives described by Formula I, where P¹ is a protecting group and X¹, X², A, M, and Q are as defined above, includes

(a) contacting an amino acid having Formula X with a reagent capable of forming a protecting group on the amino group of the amino acid to form a compound with Formula XI

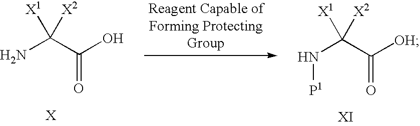

(b) activating of the carboxylic acid of Formula XI and contacting it with an amino compound of the formula H₂N-M-Q to form a compound of Formula XII

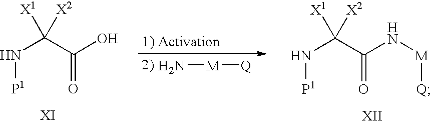

and

(c) removing the amino protecting group of the compound of Formula XII and contacting the resulting free amine with an isocyanate having A to form a compound of Formula I

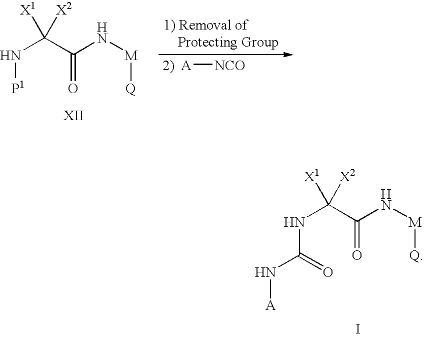

In yet another embodiment of the present invention, a method for preparing amino acid derivatives described by Formula I is provided. The method for preparing amino acid derivatives described by Formula I, where X¹, X², A, M, and Q are as defined above, includes

(a) contacting a compound of Formula XVII with a bromoacetyl chloride of the Formula XX to form a compound of Formula XXI

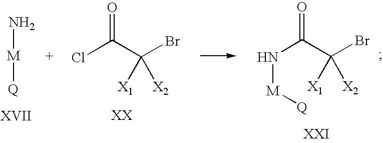

(b) contacting a compound of Formula XXI with an amine of Formula XXII to form a compound of Formula XXIII

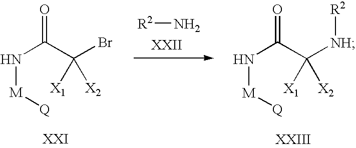

(c) contacting a compound of Formula XXXI with an isocyanate having A to form a compound of Formula I

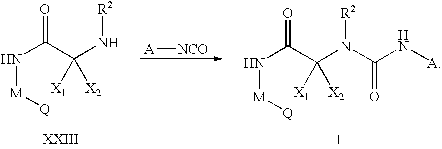

In yet another embodiment of the present invention, a method for preparing amino acid derivatives described by Formula I is provided. The method for preparing amino acid derivatives described by Formula I, where P¹ and P² are independent protecting groups and A, M, and Q are as defined above, includes

(a) base catalyzed ring opening of a compound of Formula XXVIII to form a compound of Formula XXIX

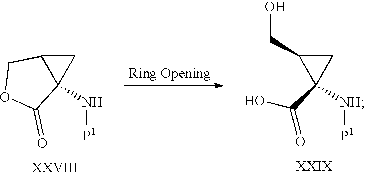

(b) contacting a compound of Formula XXIX with a reagent capable of forming a protecting group on the hydroxyl groups followed by contacting the resulting intermediate with a reagent capable of selective deprotection of the carboxylic acid hydroxyl group to form a compound with Formula XXX

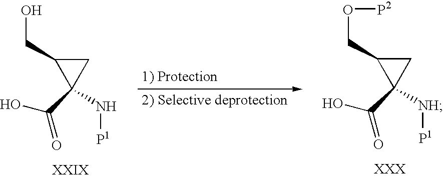

(c) activating the carboxylic acid of Formula XXX and contacting it with an amino compound of the formula XXXI to form a compound of Formula XXXII

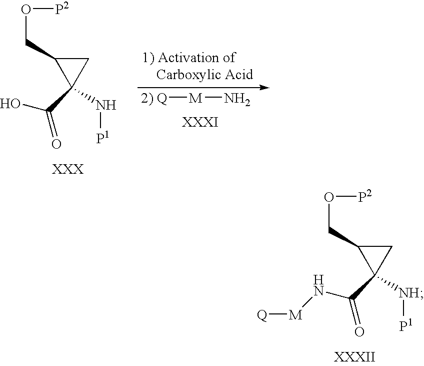

and

(d) removing the amino protecting group of the compound of Formula XXXII and contacting the resulting free amine with an isocyanate having A to from a compound of Formula I

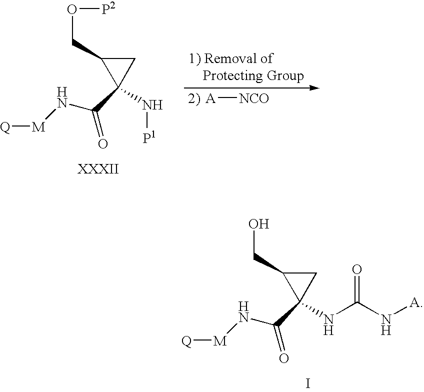

In yet another embodiment of the present invention, a method for preparing amino acid derivatives described by Formula I is provided. The method for preparing amino acid derivatives described by Formula I, where P¹ and P² are independent protecting groups and A, M, and Q are as defined above, includes

(a) contacting a compound of Formula XXXIII with a reagent capable of selectively forming a protecting group on the alcohol hydroxyl group to form a compound with Formula XXXIV

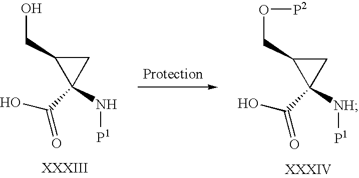

(b) activating the carboxylic acid of Formula XXXIV and contacting it with an amino compound of the formula XXXV to form a compound of Formula XXXVI

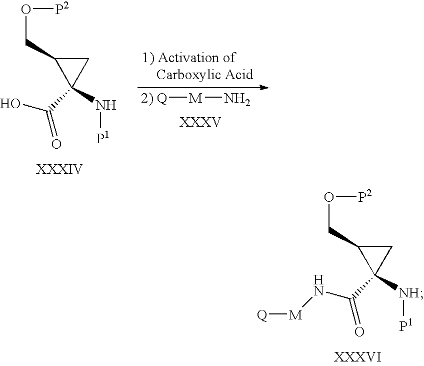

(c) removing the amino protecting group of the compound of Formula XXXVI and contacting the resulting free amine with an isocyanate having A to from a compound of Formula XXXVII

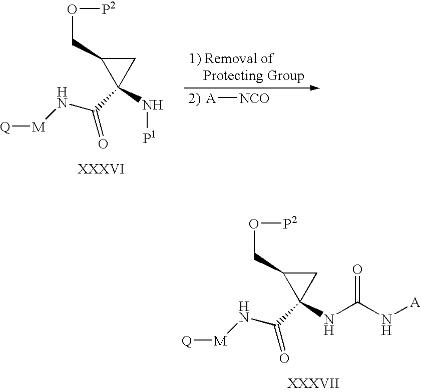

(d) removing the alcohol hydroxy protecting group of the compound of Formula XXXVII to from a compound of Formula I

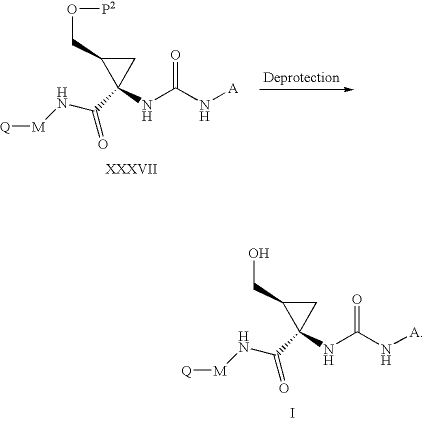

In yet another embodiment of the present invention, a method for preparing amino acid derivatives described by Formula I is provided. The method for preparing amino acid derivatives described by Formula I, where P¹ and P² are independent protecting groups and A, M, and Q are as defined above, includes

(a) contacting a compound of Formula XXXVIII with acid to form a compound of Formula XXXIX

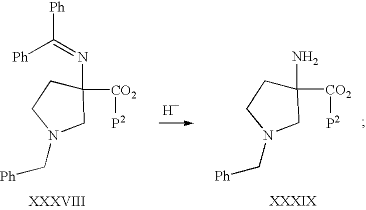

(b) contacting a compound of Formula XXXIX with a reagent capable of forming a protecting group on the amino moiety to form a compound of Formula XL

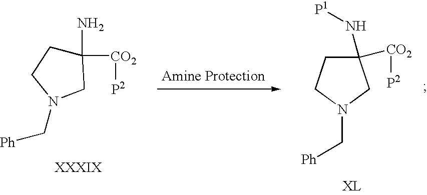

(c) contacting a compound of Formula XL with a reagent capable of forming a protecting group on the heterocycle nitrogen to form a compound of Formula XLI

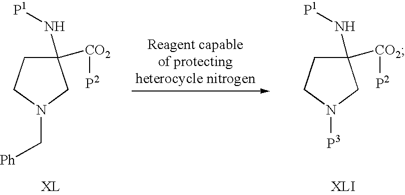

(d) contacting a compound of Formula XLI with a reagent capable of removing the protecting group of the carboxylic acid to form a compound of Formula XLII

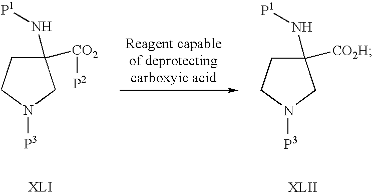

(e) activating the carboxylic acid of Formula XLII and contacting it with an amino compound of the formula XLIII to form a compound of Formula XLIV

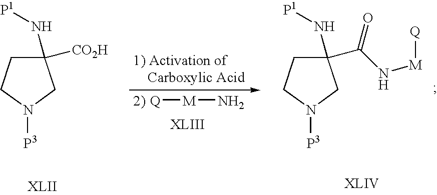

(f) removing the amino protecting group of the compound of Formula XLIV and contacting the resulting free amine with an isocyanate having A to from a compound of Formula I

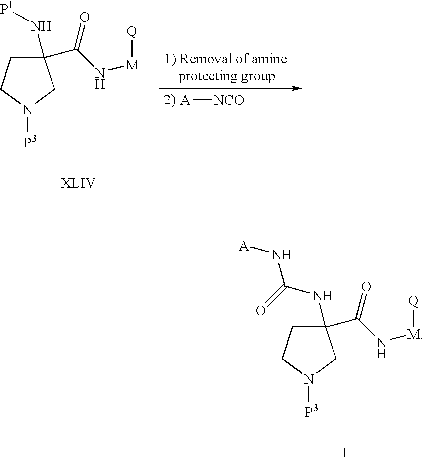

In another embodiment of the present invention, a method for preventing and treating acute, subacute, and chronic thrombotic disorders in a mammal is provided. The method of this embodiment comprises administering to such mammal a therapeutically effective amount of the compounds disclosed in the present invention.

In yet another embodiment of the present invention, pharmaceutical formulation comprising a compound of Formula I is provided.

### DETAILED DESCRIPTION OF THE INVENTION

The following definitions are used, unless otherwise described: alkyl, alkoxy, alkenyl, alkynyl, etc. denote both straight and branched groups; but reference to an individual radical such as “propyl” embraces only the straight chain radical, a branched chain isomer such as “isopropyl” being specifically referred to.

The term “halogen” or “halo” as used herein includes chlorine, fluorine, bromine, and iodine.

The term “alkyl” as used herein refers to a monovalent straight or branched hydrocarbon of from 1 to 12 carbon atoms. Alkyl groups can also be substituted with one or more of the substituents selected from lower alkoxy, lower thioalkoxy, —O(CH₂)₁₋₃CF₃, halogen, nitro, cyano, ═O, ═S, —OH, —SH, —CF₃, —OCF₃, —CO₂H, —CO₂C₁-C₆ alkyl, —NH₂, —NHC₁-C₆ alkyl, —CONR′R″, —N(C₁-C₆alkyl)₂, SO₂(C₁-C₆alkyl), SO₂ NR′″R″″, where R′, R″, R′″, and R″″ are independently alkyl, akenyl, alkynyl, aryl, or are joined together to form a 3 to 7 member ring. Examples of alkyl groups include, but are not limited to methyl, ethyl, n-propyl, isopropyl, n-butyl, sec-butyl, isobutyl, tert-butyl, n-pentyl, and n-hexyl. Examples of substituted alkyl groups include, but are not limited to, trifluoromethyl, hydroxymethyl, aminomethyl, and ethylaminomethyl.

The term “lower” as used herein refers to a group having 1 to 6 carbon atoms. For example “lower alkyl” as used herein refers to a subset of alkyl which means a straight or branched hydrocarbon radical having from 1 to 6 carbon atoms and includes, for example, methyl, ethyl, n-propyl, isopropyl, n-butyl, sec-butyl, isobutyl, tert-butyl, n-pentyl, and n-hexyl.

The term “cycloalkyl” as used herein means a monovalent hydrocarbon ring radical containing from 3 to 12 carbon atoms. Cycloalkyl rings may be unsubstituted or substituted by one or more substituents selected from lower alkyl, lower alkoxy, lower thioalkoxy, —O(CH₂)₁₋₃CF₃, halogen, nitro, cyano, ═O, ═S, —OH, —SH, —CF₃, —OCF₃, —CO₂H, —CO₂C₁-C₆ alkyl, —(CH₂)₁₋₃OC₁-C₆ alkyl, —(CH₂)₁₋₃OH, —NH₂, —NHC₁-C₆ alkyl, —CONR′R″, —N(C₁-C₆alkyl)₂, —(CH₂)₁₋₃NH₂, —(CH₂)₁₋₃ NHC₁-C₆ alkyl, —(CH₂)₁₋₃ NH(COC₁-C₆ alkyl), —(CH₂)₁₋₃N(C₁-C₆alkyl)₂, SO₂(C₁-C₆alkyl), and SO₂ NR′″R″″, where R′, R″, R′″, and R″″ are as defined above. Examples of cycloalkyl groups include, but are not limited to, cyclopropyl, cyclobutyl, cyclopentyl, cyclohexyl, cycloheptyl, cycloctyl, decalinyl, norpinanyl, and adamantly. Examples of substituted cycloalkyl groups include, but are not limited to, 2-hydroxymethylcyclopropyl, 2-aminomethylcyclopropyl, 2-acetylamino-methyl-cyclopropyl, 2-methoxymethylcyclopropyl, 2-carboxycyclopropyl, 2-acetylcyclopropyl, 3-hydroxymethylcyclobutyl, 3-aminomethylcyclobutyl, 2-methoxymethylcyclobutyl, 3-carboxycyclobutyl, 3-acetylcyclobutyl, and 3,4-dihydroxycyclopentyl.

The term “cycloalkylene” as used herein means a divalent hydrocarbon ring radical containing from 3 to 12 carbon atoms. Cycloalkylene groups may be unsubstituted or substituted with those substituents enumerated for cycloalkyl. Examples of cycloalkylene groups include, but are not limited to, cyclopropyl-1,2-diyl, cyclobutyl-1,2-diyl, cyclopentyl-1,3-diyl, cyclohexyl-1,2-diyl, cyclohexyl-1,3-diyl, cyclohexyl-1,4-diyl, cycloheptyl-1,4-diyl, and cyclooctyl-1,5-diyl.

The term “alkenyl” as used herein means a straight or branched unsaturated hydrocarbon radical containing 2 to 10 carbon atoms and at least one carbon-carbon double bond. Alkenyl groups may be unsubstituted or substituted by one or more substituents selected from lower alkyl, lower alkoxy, lower thioalkoxy, —O(CH₂)₁₋₃CF₃, halogen, nitro, cyano, ═O, ═S, —OH, —SH, —CF₃, —OCF₃, —CO₂H, —CO₂C₁-C₆ alkyl, —(CH₂)₁₋₃OC₁-C₆ alkyl, —(CH₂)₁₋₃OH, —NH₂, —NHC₁-C₆ alkyl, CONR′R″, —N(C₁-C₆alkyl)₂, SO₂(C₁-C₆alkyl), and SO₂ NR′″R″″ where R′, R″, R′″, and R″″ are as defined above. Examples of alkenyl groups include, but are not limited to, ethenyl, 1-propenyl, 2-propenyl, 1-butenyl, 2-butenyl, 1-pentenyl, 2-pentenyl, 3-methyl-3-butenyl, 1-hexenyl, 2-hexenyl, 3-hexenyl, 3-heptenyl, 1-octenyl, 1-nonenyl, 1-decenyl, 1-undecenyl, and 1-dodecenyl.

The term “cycloalkenyl” as used herein means a monovalent hydrocarbon ring radical having 3 to 12 carbon atoms and at least one carbon-carbon double bond in the ring system. Cycloalkenyl groups may be unsubstituted or substituted with those substituents enumerated for cycloalkyl. Examples of cycloalkenyl groups include, but are not limited to, cyclopentenyl, cyclohexenyl, and the like.

The term “cycloalkenylene” as used herein means a divalent hydrocarbon ring radical having 3 to 12 carbon atoms and at least one carbon-carbon double bond in the ring system. Cycloalkenylene groups may be unsubstituted or substituted with those substituents enumerated for cycloalkyl. Examples of cycloalkenylene groups include, but are not limited to, cyclopentene-1,3-diyl, cyclopentene-3,5-diyl, cyclopentene-1,2-diyl, cyclohexene-1,2-diyl, cyclohexenel,3-diyl, cyclohexene-1,4-diyl, cyclohexene-3,6-diyl, and cyclohexene-4,5-diyl.

The term “alkynyl” as used herein means a straight or branched monovalent hydrocarbon radical having at least one carbon-carbon triple bond. Alkynyl groups may be unsubstituted or substituted with those substituents enumerated for alkenyl. Examples of alkynyl groups include, but are not limited to, ethynyl, propynyl, 3-butyn-1-yl, and 5-hexyn-1-yl.

The term “aryl” as used herein means monovalent unsaturated aromatic carbocyclic radicals having a single ring, such as phenyl, or multiple condensed rings, such as naphthyl or anthryl. Aryl groups may be unsubstituted or substituted with 1 to 5 substituents selected from lower alkyl, lower alkoxy, lower thioalkoxy, —O(CH₂)₁₋₃CF₃, halogen, nitro, cyano, —OH, —SH, —CF₃, —OCF₃, —CO₂H, —CO₂C₁-C₆ alkyl, —(CH₂)₁₋₃OC₁-C₆ alkyl, —(CH₂)₁₋₃OH, —NH₂, —NHC₁-C₆ alkyl, —CONR′R″, —N(C₁-C₆alkyl)₂, SO₂(C₁-C₆alkyl), and SO₂ NR′″R″″, where R′, R″, R′″, and R″″ are as defined above. Examples of aryl groups include, but are not limited to, phenyl, naphthyl, and anthryl. Examples of substituted aryl groups include, but are not limited to, 2-chlorophenyl, 3-chlorophenyl, 4-chlorophenyl, 2-methylphenyl, 3-methylphenyl, 4-methylphenyl, 2-methoxyphenyl, 3-methoxyphenyl, 4-methoxyphenyl, 2-methanesulfonylphenyl, 3-methanesulfonylphenyl, 4-methanesulfonylphenyl, 2-sulfamoylphenyl, 3-sulfamoylphenyl, and 4-sulfamoylphenyl.

The term “arylene” as used herein means divalent unsaturated aromatic carbocyclic radicals having a single ring, such as phenylene, or multiple condensed rings, such as naphthylene or anthrylene. Arylene groups may be unsubstituted or substituted with those substituents enumeratred for aryl. Examples of aryl groups include, but are not limited to, phenylene-1,2-diyl, phenylene-1,3-diyl, phenylene-1,4-diyl, naphthalene-2,7-diyl, naphthalene-2,6-diyl anthracene-1,4-diyl, anthracene-2,6-diyl, and anthracene-2,7-diyl. Examples of substituted aryl groups include, but are not limited to, 2-fluoro-phenylene-1,3-diyl, 2-fluoro-phenylene-1,4-diyl, 2-chloro-phenylene-1,3-diyl, 2-chloro-phenylene-1,4-diyl, 2-methyl-phenylene-1,3-diyl, 2-methyl-phenylene-1,4-diyl, 2-trifluoromethyl-phenylene-1,3-diyl, and 2-trifluoromethyl-phenylene-1,4-diyl.

The term “heteroaryl” as used herein means an aromatic cyclic or polycyclic ring system having from 1 to 4 heteroatoms independently selected from N, O, and S. Heteroaryl groups may be unsubstituted or substituted with one or more groups enumerated for aryl. Examples of heteroaryl include, but are not limited to, 2- or 3-thienyl, 2- or 3-furanyl, 2- or 3-pyrrolyl, 1-, 2-, 3-, 4-, or 5-imidazolyl, 1-, 2-, 3-, 4-, or 5-pyrazolyl, 2-, 4-, or 5-thiazolyl, 3-, 4-, or 5-isothiazolyl, 2-, 4-, or 5-oxazolyl, 3-, 4-, or 5-isoxazolyl, 3- or 5-1,2,4-triazolyl, 4- or 5-1,2,3-triazolyl, tetrazolyl, 2-, 3-, or 4-pyridinyl, 3-, 4-, or 5-pyridazinyl, 2-pyrazinyl, 2-, 4-, or 5-pyrimidinyl, 2-, 3-, 4-, 5-, 6-, 7-, or 8-quinolinyl, 1-, 3-, 4-, 5-, 6-, 7-, or 8-isoquinolinyl, 2-, 3-, 4-, 5-, 6-, or 7-indolyl, 2-, 3-, 4-, 5-, 6-, or 7-benzo[b]thienyl, 2-, 4-, 5-, 6-, or 7-benzoxazolyl, 2-, 4-, 5-, 6-, or 7-benzimidazolyl, 2-, 4-, 5-, 6-, or 7-benzothiazolyl. Examples of substituted heteroaryl include, but are not limited to, 5-chloro-2-pyridyl, 4-methoxypyridyl, 5-fluoro-2-pyridyl, 2-oxo-2H-pyridin-1-yl, 4-oxo-1H-pyridin-1-yl, 5-methyl-pyrazol-1-yl, 3-methyl-pyrazol-1-yl, 3,5-dimethyl-pyrazol-1-yl, 2-methyl-imidazol-1-yl, 3-methanesulfonyl-2-pyridyl, and 3-sulfamoyl-2-pyridyl.

The term “heteroarylene” as used herein means a divalent aromatic cyclic or polycyclic ring system having from 1 to 4 heteroatoms independently selected from N, O, and S. Heterorylene groups may be unsubstituted or substituted with those substituents enumeratred for heteroaryl. Examples of aryl groups include, but are not limited to, furan-2,5-diyl, thiophene-2,4-diyl, 1,3-thiazole-2,4-diyl, 1,3-thiazole-2,5-diyl, pyridine-2,4-diyl, pyridine-2,3-diyl, pyridine-2,5-diyl, pyrimidine-2,4-diyl, and pyrimidine-2,5-diyl.

The term “heterocycloalkyl” as used herein means a nonaromatic monovalent ring having from 4 to 8 members, of which, up to 4 are heteroatoms such as N, O and S for example. Heterocycloalkyl groups may be unsubstituted or substituted with those substituents enumeratred for cycloalkyl. Examples of heterocycloalkyl groups include, but are not limited to, 2- or 3-tetrahydrothieno, 2- or 3-tetrahydrofurano, 1-, 2- or 3-pyrrolidino, 2-, 4-, or 5-thiazolidino, 2-, 4-, or 5-oxazolidino, 2-, 3-, or 4-piperidino, N-morpholinyl or N-thiamorpholinyl. Examples of substituted heterocycloalkyl groups include, but are not limited to, 1-methyl-pyrrolidin-3-yl, 1-acetyl-pyrrolidin-3-yl, 1-methyl-piperidin-4-yl, 1-acetyl-piperidin-4-yl, 1-methyl-azetidin-3-yl, 1-acetyl-azetidin-3-yl, and 2-oxo-piperidin-1-yl.

The term “heterocycloalkylene” as used herein means a nonaromatic divalent ring having from 4 to 8 members, of which, up to 4 are heteroatoms such as N, O and S for example. Heterocycloalkylene groups may be unsubstituted or substituted with those substituents enumeratred for cycloalkyl. Examples of heterocycloalkylene groups include, but are not limited to, tetrahydrothiene-2,4-diyl, tetrahydrofuran-2,4-diyl, pyrrolidine-2,4-diyl, thiazolidine-2,4-diyl, oxazolidine-2,4-diyl, piperidine-2,4-diyl, tetrahydrothiene-2,5-diyl, tetrahydrofuran-2,5-diyl, pyrrolidine-2,5-diyl, thiazolidine-2,5-diyl, oxazolidine-2,5-diyl, piperidine-2,5-diyl, morpholine-3,6-diyl, morpholine-2,5-diyl, morpholine-2,4-diyl, thiamorpholine-3,6-diyl, thiamorpholine-2,5-diyl, and thiamorpholine-2,4-diyl.

The term “heterocycloalkenyl” as used herein means a nonaromatic monovalent ring having from 4 to 8 members, of which, up to 4 are heteroatoms such as N, O or S for example, and at least one carbon-carbon double bond. Heterocycloalkenyl groups may be unsubstituted or substituted with those substituents enumeratred for cycloalkyl. Examples of heterocycloalkenyl groups include, but are not limited to, 2,5-dihydro-pyrrole-1-yl, 2,5-dihydro-pyrrole-2-yl, 2,5-dihydro-pyrrole-3-yl, 1,2,3,4-tetrahydropyridine-1-yl, 1,2,3,4-tetrahydropyridine-2-yl, 1,2,3,4-tetrahydropyridine-3-yl, 3,4-dihydro-[1,4]oxazine-2-yl, and 3,4-dihydro-[1,4]oxazine-5-yl. Examples of substituted heterocycloalkenyl groups include, but are not limited to, 3-methyl-2,5-dihydro-pyrrole-2-yl, 5-methyl-2,5-dihydro-pyrrole-2-yl, 3-hydroxy-2,5-dihydro-pyrrole-2-yl, 5-hydroxy-2,5-dihydro-pyrrole-2-yl, 2-methyl-1,2,3,4-tetrahydropyridine-1-yl, 3-methyl-1,2,3,4-tetrahydropyridine-1-yl, 2-hydroxyl-1,2,3,4-tetrahydropyridine-1-yl, 3-hydroxyl-1,2,3,4-tetrahydropyridine-1-yl, 2-methyl-3,4-dihydro-[1,4]oxazine-5-yl, and 5-methyl-3,4-dihydro-[1,4]oxazine-2-yl.

The term “heterocycloalkenylene” as used herein means a nonaromatic divalent ring having from 4 to 8 members, of which, up to 4 are heteroatoms such as N, O or S for example, and at least one carbon-carbon double bond. Heterocycloalkenylene groups may be unsubstituted or substituted with those substituents enumeratred for cycloalkyl. Examples of heterocycloalkylene groups include, but are not limited to, 2,5-dihydro-pyrrole-2,5-diyl, 2,5-dihydro-pyrrole-3,4-diyl, 1,2,3,4-tetrahydropyridine-2,4-diyl, 1,2,3,4-tetrahydropyridine-2,5-diyl, 1,2,3,4-tetrahydropyridine-1,4-diyl, 3,4-dihydro-[1,4]oxazine-2,5-diyl, and 3,4-dihydro-[1,4]oxazine-2,3-diyl.

The term “heterocycloalkylalkyl” as used herein means a “heterocycloalkyl” group, as defined above, terminating in a “alkyl” group, as defined above, which is the point of attachment. Examples of heterocycloalkylalkyl groups include, but are not limited to, 2-(4-methyl-piperazin-1-yl)-ethyl, 2-(1-methyl-pyrrolidin-3-yl)ethyl, 2-morpholin-4-yl-ethyl, and 2-thiomorpholin-4-yl-ethyl.

The term “cycloalkylalkyl” as used herein means a “cycloalkyl” group, as defined above, terminating in a “alkyl” group, as defined above, which is the point of attachment. Examples of cycloalkylalkyl include, but are not limited to, 2-hydroxymethylcyclopropylmethyl, cyclopropylmethyl, cyclobutylmethyl, cyclopentylmethyl, cyclohexylmethyl, 2-cyclopropylethyl, 2-cyclohexylethyl, 2-cyclohexylpropyl, 3-cyclohexylpropyl, 2-cyclohexylbutyl, 4-cyclohexylbutyl, 2-cyclopentylethyl, 2-cyclopentylpropyl, 3-cyclopentylpropyl, 2-cyclopentylbutyl, and 4-cyclopentylbutyl.

The term “aralkyl” as used herein means an “aryl” group, as defined above, terminating in an “alkyl” group, as defined above, which is the point of attachment. Examples of aralkyl groups include, but are not limited to, benzyl, phenethyl, 4-methoxy-benzyl, and 3-phenylpropyl.

The term “heteroaralkyl” as used herein means a “heteroaryl” group, as defined above, terminating in an “alkyl” group, as defined above, which is the point of attachment. Examples of heteroaralkyl include, but are not limited to, 4-methoxy-1-pyridin-3-ylmethyl, 2-pyridinylmethyl, 3-pyridinylmethyl, 4-pyridinylmethyl, 3-(2-pyridinyl)-propyl, and thienylmethyl.

The term “alkoxy” as used herein means —O-alkyl groups wherein “alkyl” is defined above.

The term “thioalkoxy” as used herein means —S-alkyl groups wherein “alkyl” is defined above.

The symbol “ [symbol] ” indicates the point of attachment.

The term “patient” means all mammals including humans. Examples of patients include humans, cows, dogs, cats, goats, sheep, pigs, and rabbits.

A “therapeutically effective amount” is an amount of a compound of the present invention that when administered to a patient ameliorates a symptom of thrombotic disorders, venous thrombosis, arterial thrombosis, pulmonary embolism, myocardial infarction, cerebral infarction, restenosis, cancer, angina, diabetes, atrial fibrillation, or heart failure. A therapeutically effective amount of a compound of the present invention can be easily determined by one skilled in the art by administering a quantity of a compound to a patient and observing the result. In addition, those skilled in the art are familiar with identifying patients having thrombotic disorders, venous thrombosis, arterial thrombosis, pulmonary embolism, myocardial infarction, cerebral infarction, restenosis, cancer, angina, diabetes, atrial fibrillation, or heart failure.

The term “pharmaceutically acceptable salts, esters, amides, and prodrugs” as used herein refers to those carboxylate salts, amino acid addition salts, esters, amides, and prodrugs of the compounds of the present invention which are, within the scope of sound medical judgment, suitable for use in contact with the tissues of patients without undue toxicity, irritation, allergic response, and the like, commensurate with a reasonable benefit/risk ratio, and effective for their intended use, as well as the zwitterionic forms, where possible, of the compounds of the invention. The term “salts” refers to the relatively non-toxic, inorganic and organic acid addition salts of compounds of the present invention. These salts can be prepared in situ during the final isolation and purification of the compounds or by separately reacting the purified compound in its free base form with a suitable organic or inorganic acid and isolating the salt thus formed. Representative salts include the hydrobromide, hydrochloride, sulfate, bisulfate, nitrate, acetate, oxalate, valerate, oleate, palmitate, stearate, laurate, borate, benzoate, lactate, phosphate, tosylate, citrate, maleate, fumarate, succinate, tartrate, naphthylate mesylate, glucoheptonate, lactobionate, and laurylsulphonate salts, and the like. These may include cations based on the alkali and alkaline earth metals, such as sodium, lithium, potassium, calcium, magnesium, and the like, as well as non-toxic ammonium, quaternary ammonium, and amine cations including, but not limited to ammonium, tetramethylammonium, tetraethylammonium, methylamine, dimethylamine, trimethylamine, triethylamine, ethylamine, and the like. (See, for example, Berge S. M., et al., “Pharmaceutical Salts,” J. Pharm. Sci., 1977;66:1-19, which is incorporated herein by reference.) The free base form may be regenerated by contacting the salt form with a base. While the free base more may differ from the salt form in terms of physical properties, such as solubility, the salts are equivalent to their respective free bases for the purposes of the present invention.

Examples of pharmaceutically acceptable, non-toxic esters of the compounds of this invention include C₁-C₆ alkyl esters wherein the alkyl group is a straight or branched chain. Acceptable esters also include C₅-C₇ cycloalkyl esters as well as arylalkyl esters such as, but not limited to benzyl. C₁-C₄ alkyl esters are preferred. Esters of the compounds of the present invention may be prepared according to conventional methods.

Examples of pharmaceutically acceptable, non-toxic amides of the compounds of this invention include amides derived from ammonia, primary C₁-C₆ alkyl amines and secondary C₁-C₆ dialkyl amines wherein the alkyl groups are straight or branched chain. In the case of secondary amines, the amine may also be in the form of a 5- or 6-membered heterocycle containing one nitrogen atom. Amides derived from ammonia, C₁-C₃ alkyl primary amines and C₁-C₂ dialkyl secondary amines are preferred. Amides of the compounds of the invention may be prepared according to conventional methods.

The term “prodrug” is an inactive derivative of a drug molecule that requires a chemical or an enzymatic biotransformation in order to release the active parent drug in the body. Prodrugs include any covalently bonded carrier which releases the active parent drug according to Formula I in vivo. A thorough discussion is provided in T. Higuchi and V. Stella, “Pro-drugs as Novel Delivery Systems,” Vol. 14 of the A.C.S. Symposium Series, and in Bioreversible Carriers in Drug Design, ed. Edward B. Roche, American Pharmaceutical Association and Pergamon Press, 1987, both of which are hereby incorporated by reference. Examples of prodrugs include acetates, formates, benzoate derivatives of alcohols, and amines present in compounds of Formula I.

The present invention provides a compound having Formula I:

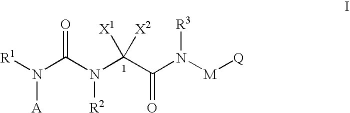

and pharmaceutically acceptable salts thereof, where:

X¹ and X² are hydrogen, alkyl, alkenyl, alkynyl, cycloalkyl, aryl, heteroaryl, aralkyl, cycloalkylalkyl, —(CH₂)ₘ-halogen, —(CH₂)ₘ-heteroaryl, —(CH₂)ₘ—SOR³, —(CH₂)ₘ—OCOR³, —(CH₂)ₘ—OSO₂R³, —(CH₂)ₘ—OSO₂NR⁴R⁵, —(CH₂)ₘ—NR⁶COR³, —(CH₂)ₘNR⁶SO₂R³, —(CH₂)ₘ—NR³SO₂NR⁴R⁵, —(CH₂)ₘNR⁴R⁵, —(CH₂)ₘOR³, —CN, —NO₂, —CF₍₃₋ₙ₎Hₙ, —(CH₂)ₘ—O(CH₂)ₘR³, —(CH₂)ₘ—O(CH₂)ₘ—OR³, —(CH₂)ₘ—O(CH₂)ₘ—NR⁴R⁵, —(CH₂)ₘR³, —(CH₂)ₘCO₂R³, —(CH₂)ₘCOR³, —(CH₂)ₘCONR⁴R⁵, —(CH₂)ₘNR⁶COR³, —(CH₂)ₘNR⁶CONR⁴R⁵, —(CH₂)ₘSO₂R³, —(CH₂)ₘSO₂NR⁴R⁵,

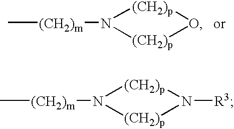

or are joined together to form a substituted or unsubstituted three to eight member ring wherein 0 to 3 atoms of the ring are heteroatoms;

A is aryl, arylcycloalkyl, heteroaryl, heteroarylcycloalkyl, cycloalkyl, or cycloalkenyl;

M is arylene, heteroarylene, cycloalkylene, heterocycloalkylene, cycloalkenylene or heterocycloalkenylene;

Q is —CONR⁴R⁵, aryl, heteroaryl, cycloalkyl, cycloalkenyl, heterocycloalkyl, or heterocycloalkenyl;

R¹ is hydrogen, alkyl, aryl, heteroaryl or alkenyl;

R² is hydrogen, alkyl, aryl, heteroaryl, alkenyl, cycloalkyl, cycloalkylalkyl, aralkyl, heteroaralkyl, heterocycloalkylalkyl, carboxy, —(CH₂)ₘNR⁴R⁵, —(CH₂)ₘOR³, —(CH₂)ₘSR³, —(CH₂)ₘCONR⁴R⁵, or —(CH₂)ₘNR⁶COR³;

R³ is hydrogen, alkyl, aryl, heteroaryl, alkenyl, alkynyl, cycloalkyl, cycloalkylalkyl, aralkyl, or heteroarylalkyl;

R⁶ is hydrogen, alkyl, aryl, heteroaryl, alkenyl, alkynyl, cycloalkyl, cycloalkylalkyl, aralkyl, or heteroarylalkyl;

R⁴ and R⁵ are each independently hydrogen, alkyl, aryl, heteroaryl, alkenyl, alkynyl, cycloalkyl, cycloalkylalkyl, aralkyl, heteroarylalkyl,

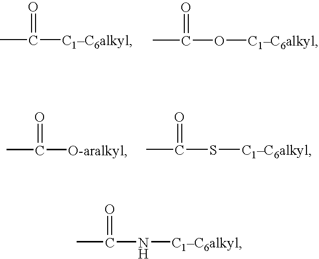

or joined together to form a 3 to 8 member ring;

m is 0 to 8;

n is 0 to 2; and

p is 1 to 3;

with the proviso that when R¹ and R² are H, neither X¹ nor X² is H.

Examples of compounds of Formula I include those where A is aryl or heteroaryl. For example, compounds of Formula I where A is aryl or heteroaryl, include those where A is

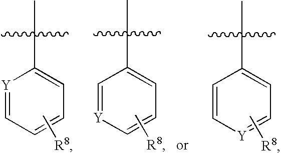

where Y is CH or N; and R⁸ is hydrogen, halo, or C₁-C₆ alkyl.

Other examples of compounds of Formula I include those where A is

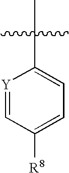

where Y is CH or N; and R⁸ is hydrogen, Cl, Br, or F.

Additional examples of compounds of Formula I include those where M is arylene or heteroarylene. For example, compounds of Formula I where M is arylene or heteroarylene include those where M is

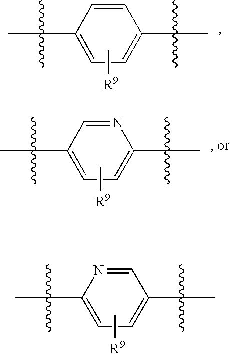

where R⁹ is hydrogen, halo, or C₁-C₆ alkyl. It is. understood, that the divalent heteroaryl groups provided above for M, are drawn so that the left side is bonded to the amide in Formula I, and the right side is bonded to the Q group.

Other examples of compounds of Formula I include those where R⁹ is in an ortho position relative to the bond to the amide nitrogen, as illustrated below:

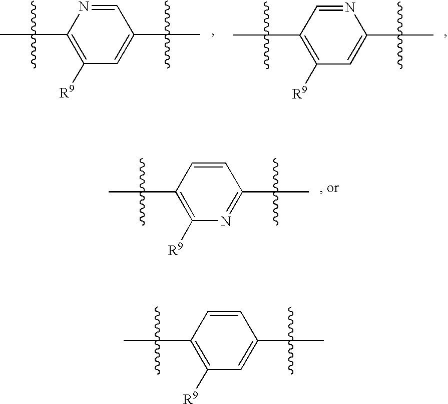

where R⁹ is hydrogen, methyl, trifluoromethyl, Cl, Br, or F.

Further examples of compounds of Formula I include those where Q is —CONR⁴R⁵, aryl, heterocycloalkyl, or heteroaryl, where R⁴ and R⁵ are as defined above. For example, compounds of Formula I where Q is —CONR⁴R⁵, aryl, heterocycloalkyl, or heteroaryl include those where Q is

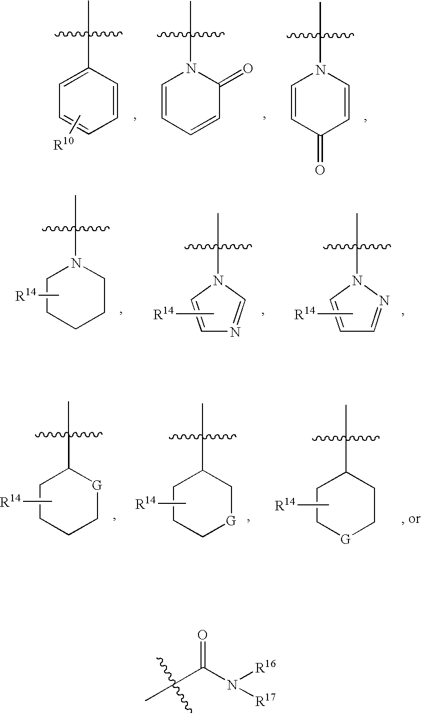

where G is O S, NH, N—(C₁-C₆alkyl),

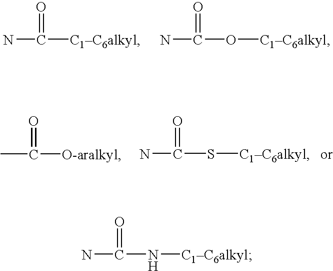

R¹⁴ is hydrogen, halo, C₁-C₆ alkyl, —SO₂NR¹²R¹³, —SO₂alkyl or oxo; R¹⁶ and R¹⁷ are independently hydrogen, C₁-C₆ alkyl, or are joined together to form a saturated or unsaturated 3 to 8 membered ring; and R¹⁰ is hydrogen, halo, C₁-C₆ alkyl, —SO₂NR¹²R¹³, or —SO₂alkyl, C₁-C₆ alkyl, where R¹² and R¹³ are independently hydrogen, C₁-C₆ alkyl, or are joined together to form a saturated 5 to 7 membered ring.

Other examples of compounds of Formula I include those where R¹⁰ and R¹⁴ are in an ortho position relative to the bond to the M group, as illustrated below:

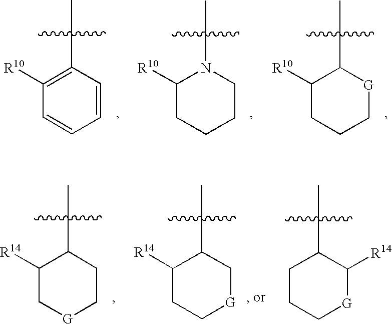

where G is NH, N—(C₁-C₆alkyl) or

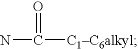

R¹⁴ is hydrogen, —SO₂NR¹²R¹³, —SO₂alkyl or oxo; and R¹⁰ is hydrogen, Cl, Br, F, —SO₂NR¹²R¹³, or —SO₂alkyl, where R¹² and R¹³ are independently hydrogen, or C-C₆ alkyl.

Further examples of compounds of Formula I include those where X¹ and X² are alkyl, —(CH₂)ₘOR³, alkenyl, or —CH₂—NR⁷R^{7′} where R⁷ and R^{7′} are independently hydrogen, —C₁-C₆ alkyl

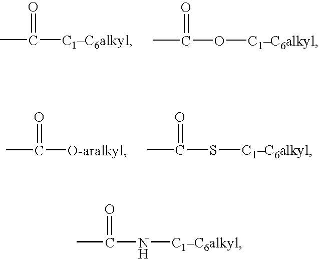

For example, compounds of Formula I where X¹ and X² are alkyl, —(CH₂)ₘOR³, —(CH₂)ₘN R⁷R^{7′} or alkenyl include those where X¹ and X² are methyl, —CH₂—OH, —CH₂NH₂, —CH₂N(CH₃)₂, and —CH₂N(CH₂CH₃)₂.

Yet further examples of compounds of Formula I include those where X¹ and X² are the same. For example, compounds of Formula I where X¹ and X² are the same include those where X¹ and X² are both hydrogen, methyl, —CH₂OH, —CH₂NH₂, —CH₂N(CH₃)₂, and —CH₂N(CH₂CH₃)₂.

Examples of compounds of Formula I where X¹ and X² are joined together to form a substituted or unsubstituted three to eight member ring wherein 0 to 3 atoms of the ring are heteroatoms include those where X¹ and X² together form a cyclopropyl, cyclobutyl, cyclopentyl, cyclohexyl, or cyclopentenyl ring including the carbon atom at position 1, or together are

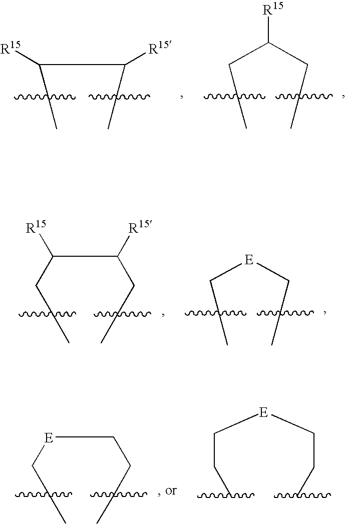

where R¹⁵ and R^{15′} are independently hydrogen, —(CH₂)₁₋₆—OH, —(CH₂)₁₋₆—O—C₁-C₆ alkyl, —(CH₂)₁₋₆—NH₂, —COOH, or —OH; and E is O, S, or NR¹⁶ where R¹⁶ is hydrogen, —C₁-C₆ alkyl,

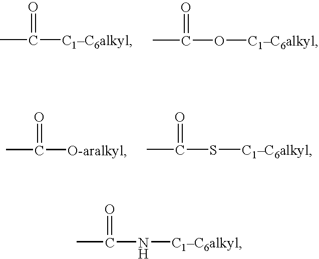

It is understood, that the divalent moieties provided above when X¹ and X² together form a ring, are drawn so that both are bonded to the carbon atom at the 1-position in order to form the ring.

In yet further examples of compounds of the Formula I, are those where R² is alkyl, aryl, heteroaryl, cycloalkyl, cycloalkylalkyl, aralkyl, heteroaralkyl, heterocycloalkylalkyl, carboxy, —(CH₂)ₘNR⁴R⁵, —(CH₂)ₘOR³, —(CH₂)ₘSR³, —(CH₂)ₘCONR⁴R⁵, or —(CH₂)ₘNR⁶COR³. For example, compounds of Formula I where R² is alkyl, aryl, heteroaryl, cycloalkyl, cycloalkylalkyl, aralkyl, heteroaralkyl, heterocycloalkylalkyl, carboxy, —(CH₂)ₘNR⁴R⁵, —(CH₂)ₘOR³, —(CH₂)ₘSR³, —(CH₂)ₘCONR⁴R⁵, or —(CH₂)ₘNR⁶COR³ include those where R² is C₁-C₆ alkyl, phenyl, pyridyl, cyclopropyl, cyclopropylmethyl, cyclobutylmethyl, cyclopentylmethyl, cyclohexylmethyl, 2-cyclopropylethyl, 2-cyclopentylethyl, benzyl, 2-pyridinylmethyl, 3-pyridinylmethyl, 4-pyridinylmethyl, 3-(2-pyridinyl)-propyl, thienylmethyl, 2-morpholin-4-yl-ethyl, 2-thiomorpholin-4-yl-ethyl, —(CH₂)₁₋₃NH₂, —(CH₂)₁₋₃N(C₁-C₆alkyl)₂, —(CH₂)₁₋₃NHC₁-C₆alkyl, C₆alkyl, —(CH₂)₁₋₃SC1-C₆alkyl, —(CH₂)₁₋₃ CONH₂, —(CH₂)₁₋₃ CON(C₁-C₆alkyl)₂, —(CH₂)₁₋₃ CONHC₁-C₆alkyl, and —(CH₂)₁₋₃NHCOC₁-C₆alkyl.

In yet further examples of compounds of the Formula I, are those where A is

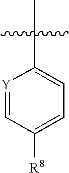

where Y is CH or N; and R⁸ is hydrogen, Cl, Br, or F;

M is

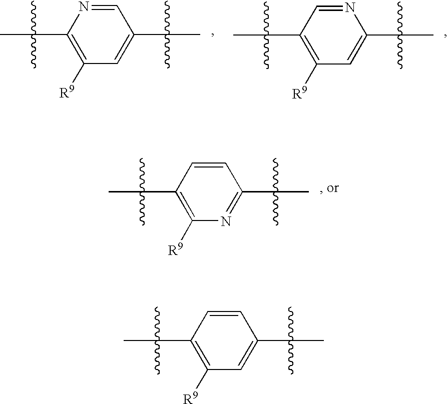

where R⁹ is hydrogen, Cl, Br, or F;

Q is

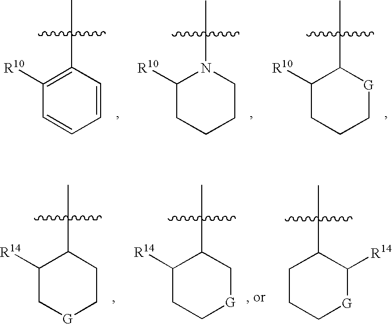

where G is NH, N—(C₁-C₆alkyl) or

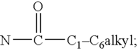

R¹⁴ is hydrogen, —SO₂NR¹²R¹³, —SO₂alkyl or oxo; and R¹⁰ is hydrogen, Cl, Br, F, —SO₂NR¹²R¹³, or —SO₂alkyl, where R¹² and R¹³ are independently hydrogen, or C₁-C₆ alkyl;

X¹ and X₂ are independently methyl, —CH₂—OH, —CH₂—NR⁷R^{7′} where R⁷ and R^{7′} are independently hydrogen or C₁-C₆ alkyl, or X¹ and X₂ together form a cyclopropyl, cyclobutyl, cyclopentyl, cyclohexyl, or cyclopentenyl ring or together are

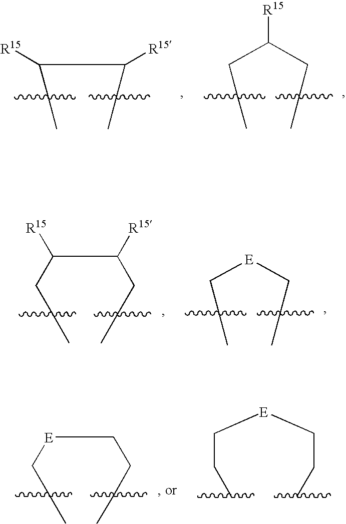

where R¹⁵ and R^{15′} are independently hydrogen, —(CH₂)₁₋₆—OH, —(CH₂)₁₋₆—O—C₁-C₆ alkyl, —(CH₂)₁₋₆—NH₂, —COOH, or —OH; and E is O, S, or NR¹⁶ where R¹⁶ is hydrogen,

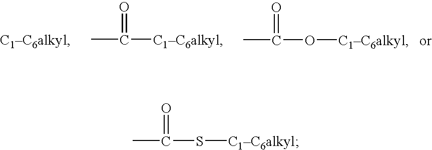

R¹ and R³are each independently hydrogen, or C₁-C₆alkyl; and

R² is hydrogen, C₁-C₆ alkyl, phenyl, pyridyl, cyclopropyl, cyclopropylmethyl, cyclobutylmethyl, cyclopentylmethyl, cyclohexylmethyl, 2-cyclopropylethyl, 2-cyclopentylethyl, benzyl, 2-pyridinylmethyl, 3-pyridinylmethyl, 4-pyridinylmethyl, 3-(2-pyridinyl)-propyl, thienylmethyl, 2-morpholin-4-yl-ethyl, 2-thiomorpholin-4-yl-ethyl, —(CH₂)₁₋₃NH₂, —(CH₂)₁₋₃N(C₁-C₆alkyl)₂, —(CH₂)₁₋₃NHC₁-C₆alkyl, —(CH₂)₁₋₃OC₁-C₆alkyl, —(CH₂)₁₋₃SC1-C₆alkyl, —(CH₂)₁₋₃CONH₂, —(CH₂)₁₋₃ CON(C₁-C₆alkyl)₂, —(CH₂)₁₋₃ CONHC₁-C₆alkyl, or —(CH₂)₁₋₃NHCOC₁-C₆alkyl.

In yet further examples of compounds of the Formula I, are those where A is

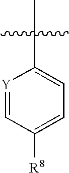

where Y is CH or N; and R⁸ is hydrogen, Cl, Br, or F;

M is

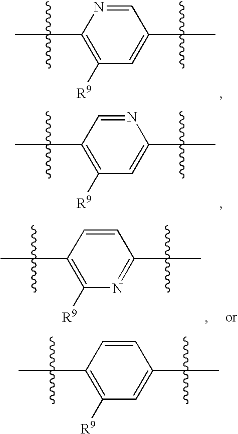

where R⁹ is hydrogen, Cl, Br, or F;

Q is

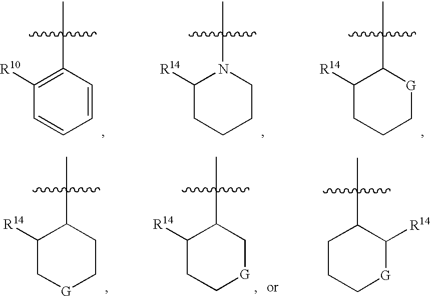

where G is NH, N—(C₁-C₆alkyl),

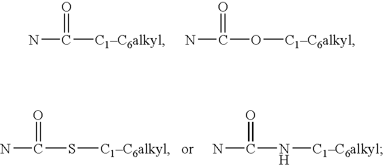

R⁴is hydrogen, —SO₂NR¹²R¹³, —SO₂alkyl or oxo; R¹⁰ is hydrogen, Cl, Br, F, —SO₂NR¹²R¹³, or —SO₂alkyl, where R¹² and R¹³ are independently hydrogen, or C₁-C₆ alkyl;

X¹ and X₂ are independently hydrogen, methyl, —CH₂—OH, —CH₂—NR⁷R^{7′} where R⁷ and R^{7′} are independently hydrogen or C₁-C₆ alkyl, or X¹ and X₂ together form a cyclopropyl, cyclobutyl, cyclopentyl, cyclohexyl, or cyclopentenyl ring or together are

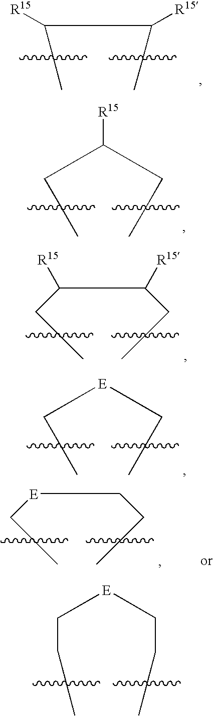

where R¹⁵ and R^{15′} are independently hydrogen, —(CH₂)₁₋₆—OH, —(CH₂)₁₋₆—O—C₁-C₆ alkyl, —(CH₂)₁₋₆—NH₂, —COOH, or —OH; and E is O, S, or NR¹⁶ where R¹⁶ is hydrogen, C₁-C₆ alkyl,

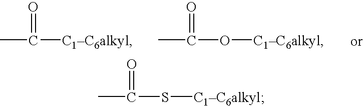

R¹ and R³are each independently hydrogen, or C₁-C₆alkyl; and

R² is C₁-C₆ alkyl, phenyl, pyridyl, cyclopropyl, cyclopropylmethyl, cyclobutylmethyl, cyclopentylmethyl, cyclohexylmethyl, 2-cyclopropylethyl, 2-cyclopentylethyl, benzyl, 2-pyridinylmethyl, 3-pyridinylmethyl, 4-pyridinylmethyl, 3-(2-pyridinyl)-propyl, thienylmethyl, 2-morpholin-4-yl-ethyl, 2-thiomorpholin-4-yl-ethyl, —(CH₂)₁₋₃NH₂, —(CH₂)₁₋₃N(C₁-C₆alkyl)₂, —(CH₂)₁₋₃NHC₁-C₆alkyl, —(CH₂)₁₋₃OC₁-C₆alkyl, —(CH₂)₁₋₃SC1-C₆alkyl, —(CH₂)₁₋₃ CONH₂, —(CH₂)₁₋₃ CON(C₁-C₆alkyl)₂, —(CH₂)₁₋₃ CONHC₁-C₆alkyl, or —(CH₂)₁₋₃NHCOC₁-C₆alkyl.

Also provided by this invention, is a method for preventing and treating acute, subacute, and chronic thrombotic disorder in a mammal comprising administering to such mammal an therapeutically effective amount of a compound of Formula I. The compounds are useful as anticoagulants for the treatment and prevention of disorders such as venous and arterial thrombosis, pulmonary embolism, and ischemic events such as myocardial infarction or cerebral infarction. These compounds also have therapeutic utility for the prevention and treatment of complications of indwelling vascular access ports and arteriovenous shunts and coagulopathies associated with cardiopulmonary bypass or other extracorporeal systems. These compounds are useful for preventing or treating unstable angina, refractory angina, intermittent claudication, disseminated intravascular coagulation, and ocular buildup of fibrin. Since thrombin and serine proteases have also been demonstrated to activate a number of different cell types, these compounds are useful for the treatment or prophylaxis of septic shock and other inflammatory responses such as acute or chronic atherosclerosis. The compounds also have utility in treating neoplasia/metastasis and neurodegenerative diseases such as Alzheimer's and Parkinson's disease. In a preferred method, the thrombotic disorder is selected from venous thrombosis, arterial thrombosis, pulmonary embolism, myocardial infarction, cerebral infarction, angina, cancer, diabetes. A further embodiment of this invention is a pharmaceutical formulation comprising a compound of Formula I administered with a diluent, excipient, or carrier thereof.

The compounds of the present invention can be administered to a patient alone or as part of a composition that contains other components such as excipients, diluents, and carriers, all of which are well-known in the art. The compounds of Formula I can be Formulated as pharmaceutical compositions and administered to a mammalian host, such as a human patient in a variety of forms adapted to the chosen route of administration, i.e., orally or parenterally, by intravenous, intramuscular, topical or subcutaneous routes. The compositions can be administered to humans and/or animals either orally, rectally, parenterally (intravenously, intramuscularly, or subcutaneously), intracisternally, intravaginally, intraperitoneally, intravesically, locally (powders, ointments, or drops), or as a buccal or nasal spray. Such compositions and preparations should contain at least 0.1% of active compound. The percentage of the compositions and preparations may, of course, be varied and may conveniently be between about 2 to about 60% of the weight of a given unit dosage form. The amount of active compound in such therapeutically useful compositions is such that an effective dosage level will be obtained.

Compositions suitable for parenteral injection may comprise physiologically acceptable sterile aqueous or nonaqueous solutions, dispersions, suspensions or emulsions, and sterile powders for reconstitution into sterile injectable solutions or dispersions. Examples of suitable aqueous and nonaqueous carriers, diluents, solvents or vehicles include water, ethanol, polyols (propyleneglycol, polyethyleneglycol, glycerol, and the like), suitable mixtures thereof, vegetable oils (such as olive oil), and injectable organic esters such as ethyl oleate. Proper fluidity can be maintained, for example, by the use of a coating such as lecithin, by the maintenance of the required particle size in the case of dispersions and by the use of surfactants.

These compositions may also contain adjuvants such as preserving, wetting, emulsifying, and dispensing agents. Prevention of the action of microorganisms can be ensured by various antibacterial and antifungal agents, for example, parabens, chlorobutanol, phenol, sorbic acid, and the like. It may also be desirable to include isotonic agents, for example sugars, sodium chloride, and the like. Prolonged absorption of the injectable pharmaceutical form can be brought about by the use of agents delaying absorption, for example, aluminum monostearate and gelatin.

Solid dosage forms for oral administration include capsules, tablets, pills, powders, and granules. In such solid dosage forms, the active compound is admixed with at least one inert customary excipient (or carrier) such as sodium citrate or dicalcium phosphate or (a) fillers or extenders, as for example, starches, lactose, sucrose, glucose, mannitol, and silicic acid; (b) binders, as for example, carboxymethylcellulose, alignates, gelatin, polyvinylpyrrolidone, sucrose, and acacia; (c) humectants, as for example, glycerol; (d) disintegrating agents, as for example, agar, calcium carbonate, potato or tapioca starch, alginic acid, certain complex silicates, and sodium carbonate; (e) solution retarders, as for example paraffin; (f) absorption accelerators, as for example, quaternary ammonium compounds; (g) wetting agents, as for example, cetyl alcohol and glycerol monostearate; (h) adsorbents, as for example, kaolin and bentonite; and (i) lubricants, as for example, talc, calcium stearate, magnesium stearate, solid polyethylene glycols, sodium lauryl sulfate, or mixtures thereof. In the case of capsules, tablets, and pills, the dosage forms may also comprise buffering agents.

Solid compositions of a similar type may also be employed as fillers in soft and hard-filled gelatin capsules using such excipients as lactose or milk sugar, as well as high molecular weight polyethyleneglycols, and the like.

Solid dosage forms such as tablets, dragees, capsules, pills, and granules can be prepared with coatings and shells, such as enteric coatings and others well-known in the art. They may contain opacifying agents, and can also be of such composition that they release the active compound or compounds in a certain part of the intestinal tract in a delayed manner. Examples of embedding compositions which can be used are polymeric substances and waxes. The active compounds can also be in micro-encapsulated form, if appropriate, with one or more of the above-mentioned excipients.

Liquid dosage forms for oral administration include pharmaceutically acceptable emulsions, solutions, suspensions, syrups, and elixirs. In addition to the active compounds, the liquid dosage forms may contain inert diluents commonly used in the art, such as water or other solvents, solubilizing agents and emulsifiers, as for example, ethyl alcohol, isopropyl alcohol, ethyl carbonate, ethyl acetate, benzyl alcohol, benzyl benzoate, propyleneglycol, 1,3-butyleneglycol, dimethylformamide, oils, in particular, cottonseed oil, groundnut oil, corn germ oil, olive oil, castor oil and sesame oil, glycerol, tetrahydrofurfuryl alcohol, polyethyleneglycols and fatty acid esters of sorbitan or mixtures of these substances, and the like.

Besides such inert diluents, the composition can also include adjuvants, such as wetting agents, emulsifying and suspending agents, sweetening, flavoring, and perfuming agents.

Suspensions, in addition to the active compounds, may contain suspending agents, as for example, ethoxylated isostearyl alcohols, polyoxyethylene sorbitol and sorbitan esters, microcrystalline cellulose, aluminum metahydroxide, bentonite, agar-agar and tragacanth, or mixtures of these substances, and the like.

Compositions for rectal administrations are preferably suppositories which can be prepared by mixing the compounds of the present invention with suitable non-irritating excipients or carriers such as cocoa butter, polyethyleneglycol, or a suppository wax, which are solid at ordinary temperatures but liquid at body temperature and therefore, melt in the rectum or vaginal cavity and release the active component.

The amount of invention compound to be utilized to prevent and treat thrombotic disorders is that amount which is effective to prevent or treat the condition without causing unacceptable side effects. Such effective amounts will be in the range of about 0.1 to about 2,000 mg per day. For a normal human adult having a body weight of about 70 kilograms, a dosage in the range of about 0.01 to about 10 mg per kilogram of body weight per day is preferable. However, the specific dosage used can vary. For example, the dosage can depended on a numbers of factors including the requirements of the patient, the severity of the condition being treated, and the pharmacological activity of the compound being used. The determination of optimum dosages for a particular patient is well-known to those skilled in the art.

When the composition is administered orally, a larger quantity of the active agent will typically be required to produce the same effect as caused with a smaller quantity given parenterally.

The compounds of the present invention may also be used as anti-coagulants in vitro or ex vivo as in the case of contact activation with foreign thrombogenic surfaces such as is found in tubing used in extracorporeal shunts. The compounds of the invention may also be used to coat the surface of such thrombogenic conduits. To this end, the compounds of the invention can be prepared as lyophilized powders, redissolved in isotonic saline or similar diluent, and added in an amount sufficient to maintain blood in an anticoagulated state.

Preparation of Compounds of the Invention

The present invention contains compounds that can be synthesized in a number of ways familiar to one skilled in organic synthesis. The compounds outlined herein can be synthesized according to the methods described below, along with methods typically utilized by a synthetic chemist, and combinations or variations of those methods which are generally known to one skilled in the art of synthetic chemistry. The synthetic route of compounds in the present invention is not limited to the methods outlined below. It is assumed one skilled in the art will be able to use the schemes outlined below to synthesize compounds claimed in this invention. Individual compounds may require manipulation of the conditions in order to accommodate various functional groups. A variety of protecting groups generally known to one skilled in the art may be required. The appropriate use and choice of protecting groups is well-known by one skilled in the art, and is not limited to the specific examples below. It is also to be understood that such groups not only serve to protect chemically reactive sites, but also to enhance solubility or otherwise change physical properties. A good general reference for protecting group preparation and deprotection is Greene, Theodora, Protective Groups in Organic Synthesis; Wiley: N.Y., USA, 1991. Purification, if necessary, can be accomplished on a silica gel column eluted with the appropriate organic solvent system. Also, reverse phase HPLC may be employed if a compound does not elute from silica gel.

Further, in regards to Schemes 1,1a, 2, 2a, 2b, 2c, 3, 3a, 4, 5, and 6, a number of general reactions such as reductions, etc. are not shown in detail but can be done by methods understood by one skilled in the art. General transformations are well-reviewed in Larock, Richard. Comprehensive Organic Transformations; Wsiley: N.Y., USA, 1999, and the series “Compendium of Organic Synthetic Methods” published by Wiley-Interscience. In general, the starting materials are obtained from commercial sources unless otherwise indicated.

Compounds of the invention can be prepared using the synthetic routes outlined in Schemes 1,1a; 2, 2a, 2b, 2c, 3, 3a, 4, 5, and 6. The routes are useful for a wide variety of starting materials with variable X¹ and X² groups, provided the appropriate protecting group is utilized if necessary. The schemes are also employed for both racemic mixtures and enantiomerically pure compounds.

The method as disclosed in Scheme 1 includes reacting an amino acid having Formula III with a reagent capable of forming a protecting group on the amino group of an amino acid to form a compound with Formula IV. In Scheme 1, P¹ is a protecting group and X¹ and X² are the same as defined above for Formula I. The carboxylic acid of Formula IV is then activated by a coupling reagent, such as BOP, HATU, EEDQ, or CDI for example, and reacted with a haloaniline or a haloaminoheterocycle, for example, to form a halide with Formula V, where Y¹ is a halogen and M is as defined above. The compound with Formula V is then subjected to a coupling reaction with a compound having Q to give a compound of Formula VI. The protecting group is then removed from compound VI and the resulting compound reacted with an isocyanate having A to from the compound with Formula I.

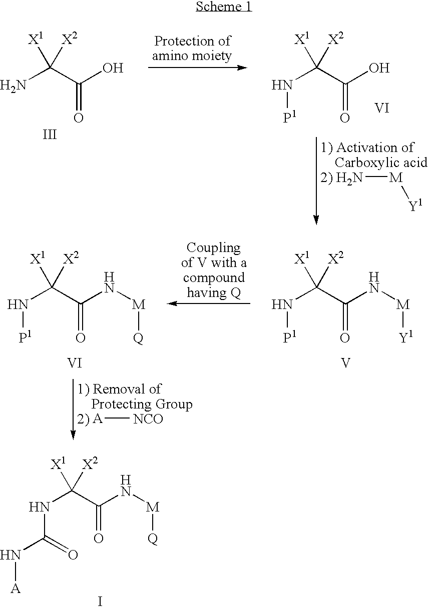

A specific example of a useful method for preparing compounds of Formula I is outlined in Scheme 1a. The synthetic route starts by protecting an amino acid with di-tert-butyl dicarbonate in tetrahydrofuran with 2M NaOH as the base. The Boc-protected amino acid VII is then combined with EEDQ, triethylamine and an appropriate bromoaniline or bromoaminoheterocycle (H₂N-M-Br) in a suitable solvent, such as chloroform for example, and heated to reflux to produce VIII. The resulting bromide is then typically subjected to metal catalyzed coupling, such as Suzuki coupling for example, with a boronic acid derivatized compound having Q, although other coupling conditions may be used. The resulting compound is then deprotected with 33% trifluoroacetic acid in dichloromethane and then reacted with an isocyanate with triethylamine in tetrahydrofuran to produce a compound with Formula I. This route is useful for compounds containing a biaryl M-Q moiety, such as a biphenyl group where Q is substituted with a tert-butylsulfonamide (tBuNHSO₂) for example. The tert-butyl group could then be removed using standard methods of amine deprotection, such as TFA in dichloromethane for example, to afford the free amine.

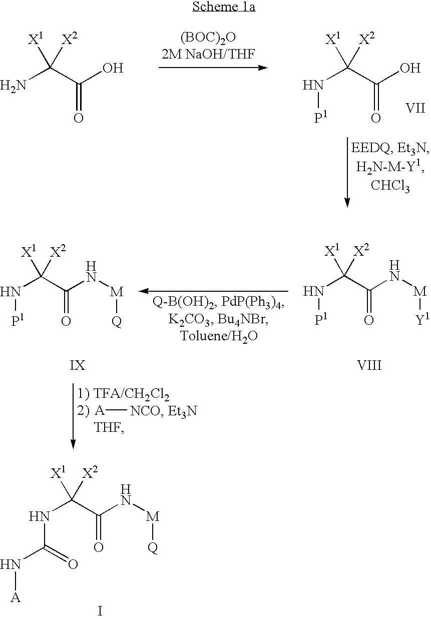

Another method of making the compounds having Formula I is provided in Scheme 2. Scheme 2 is useful for synthesizing a wide variety of M-Q groups. The chemistry is similar to Scheme 1, except that the second step introduces an aniline or aminoheterocycle that includes both the M and Q moieties, instead of a haloaniline or haloaminoaniline, and thus eliminating the coupling step to add the moiety Q. The method as disclosed in Scheme 2 includes reacting an amino acid having Formula X with a reagent capable of forming a protecting group on the amino group of an amino acid to form a compound with Formula XI. In Scheme 2, P¹ is a protecting group and X¹ and X² are the same as defined above for Formula I. The carboxylic acid XI is then activated by a coupling reagent, such as BOP, HATU, EEDQ, or CDI for example, and reacted with an aniline or an aminoheterocyclenhaving a formula H₂N-M-Q, for example, to form a compound with Formula XII. The protecting group is then removed from compound XII and the resulting compound reacted with an isocyanate having A to from the compound having Formula I.

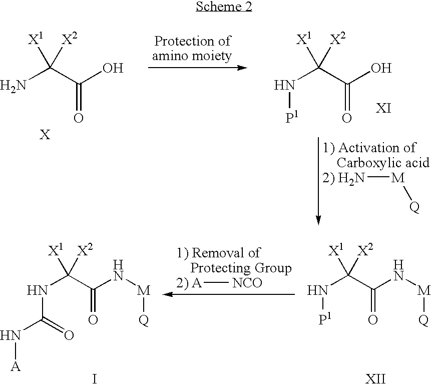

A specific example of a useful method for preparing compounds of Formula I is outlined in Scheme 2a. The synthetic route starts by protecting an amino acid XIII with di-tert-butyl dicarbonate in tetrahydrofuran with 2M NaOH as the base. The Boc-protected amino acid XIV is then combined with EEDQ, triethylamine and an appropriate aniline or aminoheterocycle in a suitable solvent, such as chloroform for example, and heated to reflux to produce XV. Compound XV is then deprotected with 33% trifluoroacetic acid in dichloromethane and then reacted with an appropriate A-isocyanate with triethylamine in tetrahydrofuran to produce compounds of Formula I. In cases where X¹ and X² of compound XV form an hydroxymethyl substituted cyclopropyl moiety, TMSI in dichloromethane can be used to deprotect the amino group. This route is also useful for compounds containing a biaryl M-Q moiety, such as a biphenyl group where Q is substituted with a tert-butylsulfonamide (tBuNHSO₂) for example. The tert-butyl group could then be removed using standard methods of amine deprotection, such as TFA in dichloromethane for example, to afford the free amine.

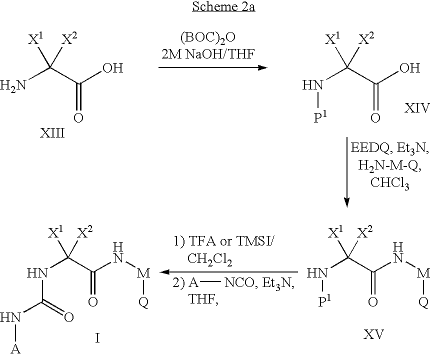

The moiety H₂N-M-Q as disclosed in Schemes 2 and 2a can be prepared as outlined in Schemes 2b and 2c. As shown in Scheme 2b the moiety H₂N-M-Q can be prepared by contacting the compound XVI with a compound having Q to produce the H₂N-M-Q moiety XVII. This reaction can be carried out by combining compound XVI, copper iodide, trans-cyclohexylamine, K₃PO₄, and Q-H in the presence of a suitable solvent, such as dioxane for example, under refluxing conditions.

As shown in Scheme 2c the moiety H₂N-M-Q can be prepared by contacting the nitro compound XVIII with a compound having Q to produce the O₂N-M-Q moiety XIX followed by the reduction of XIX to produce the H₂N-M-Q moiety XVII. An example of the preparation of moiety XII involves combining the compound XVIII with a compound having Q in the presence of a suitable solvent, such as propanol, under refluxing conditions. The resulting intermediate, XIX, is then reduced with a suitable reducing agent, such as Raney nickel for example, in a suitable solvent, such as tetrahydrofuran for example, in the presence of hydrogen gas.

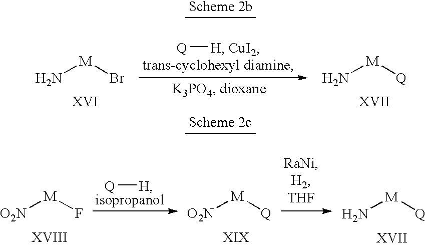

Another method of making the compounds having Formula I is provided in Scheme 3 where X¹, X², M, Q, and A are as described above for compounds of Formula I. Scheme 3 is useful for synthesizing compounds of Formula I where R² is other than hydrogen. The synthetic route starts by reacting a compound of Formula XVII, prepared according to Schemes 2b or 2c, with an appropriate bromoacetyl chloride of the Formula XX to form a compound of Formula XXI. The amide of Formula XXI is then reacted with an amine of Formula XXII, where R² is as described above for Formula I to form a compound of Formula XXIII. The compound of Formula XXIII is then reacted with an isocyanate having A to form a compound of Formula I.

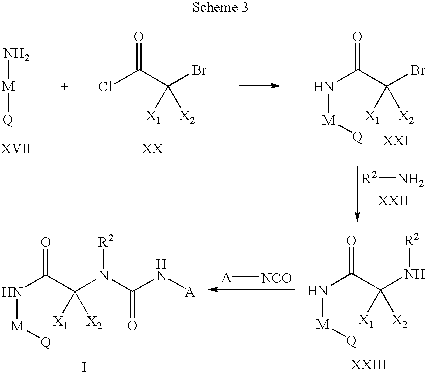

A specific example of a useful method for preparing compounds of Formula I where R² is other than hydrogen is outlined in Scheme 3a. The synthetic route starts by reacting a compound of Formula XVII, prepared according to Schemes 2b or 2c, with an appropriate bromoacetyl chloride of the Formula XXIV in the presence of a suitable base, such as triethylamine for example, and a suitable solvent, such as dichloromethane for example, to form a compound of Formula XXV. The amide of Formula XXV is then reacted with an amine of Formula XXVI, where R² is as described above for Formula I, in the presence if a base, such as diisopropylethylamine for example, and a suitable solvent, such as dichloromethane for example, followed by quenching of the reaction with a suitable quenching agent, such as benzaldehyde polystyrene resin for example, to form a compound of Formula XXVII. The compound of Formula XXVII is then reacted with an isocyanate having A, such as 4-chlorophenyl isocyanate for example, in the presence of a suitable base, such as triethylamine for example, and a suitable solvent, such as dichloromethane for example, followed by quenching of the reaction with a suitable quenching agent, such as tris-amine polystyrene resin for example, to form a compound of Formula I.

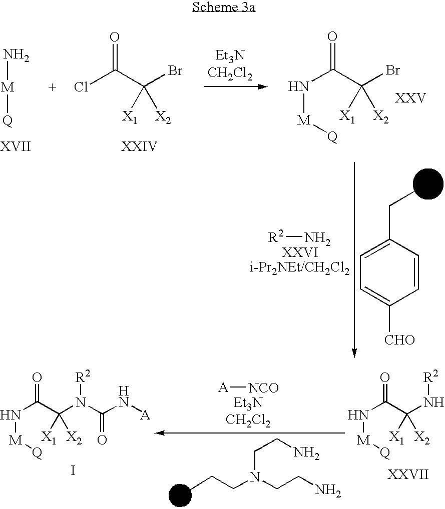

Another method of making the compounds having Formula I is provided in Scheme 4 where P¹ and P² are independent protecting groups and X¹, X², M, Q, and A are as described above for compounds of Formula I. Scheme 4 is useful for synthesizing compounds of Formula I where X¹ and X² form a hydroxymethyl substituted cyclopropyl ring. The synthetic route starts with the ring opening of a compound of Formula XXVIII (prepared according to the methods disclosed in K. Burgess et al. J.Org. Chem., 1992, 57, 5931; and D. R. Morton et al. J.Org. Chem., 1978, 57, 2101), with an appropriate base in a suitable solvent to form a compound of Formula XXIX. Suitable bases include hydroxides of the alkaline earth metals such as lithium hydroxide, for example. Suitable solvents include tetrahydrofuran, for example. The reaction can be carried out using lithium hydroxide monohydrate in a 1 to 1 mixture of THEF and water, for example. The hydroxy groups present in the compound of Formula XXIX are then protected using a suitable protecting agent, such as tert-butyldimethylsilyl chloride (TBS-Cl) for example, in the presence of a base, such as imidazole for example. The resulting bis-silylation product is then selectively deprotected to afford the compound of Formula XXX. Selective deprotection can be carried out using a variety of methods known to those of skill in the art including the use of potassium carbonate in a 3 to 1 mixture of methanol and THF for example. The carboxylic acid of Formula XXX is then activated by a coupling reagent, such as BOP, HATU, EEDQ, or CDI for example, and reacted with an amine of Formula XXXI, prepared according to Schemes 2b or 2c, to form a compound of Formula XXXII. The compound of Formula XXXII is then deprotected using a variety of methods known to those of skill in the art including the use of iodotrimethylsilane (TMS-I) in a dichloromethane. Deprotection is followed by reaction with an isocyanate having A, such as 4-chlorophenyl isocyanate for example, in the presence of a suitable solvent, such as THF for example, and a base, such as triethylamine for example, to afford the compound of Formula I.

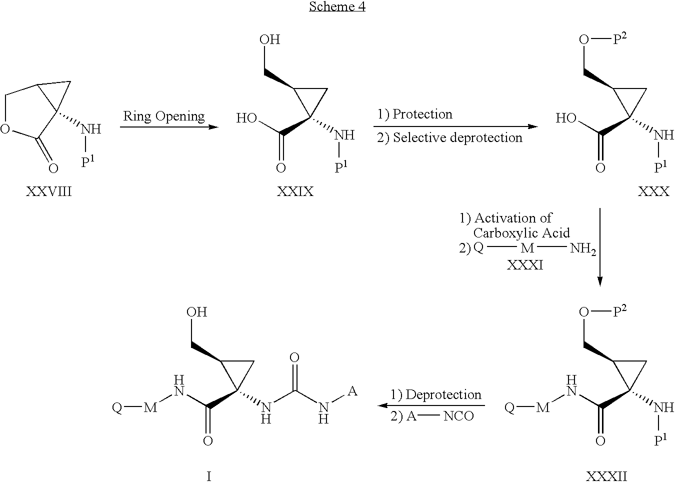

Another method of making the compounds having Formula I is provided in Scheme 5 where P¹ and P² are independent protecting groups and X¹, X², M, Q, and A are as described above for compounds of Formula I. Scheme 5 is useful for synthesizing compounds of Formula I where X¹ and X² form a hydroxymethyl substituted cyclopropyl ring. The synthetic route starts with selective protection of a compound of Formula XXXIII (prepared according to Michael C. Pirrung, Stevens E. Dunlap, Uwe P. Trinks. Helv. Chimica. Acta., 1989, 72, 1301-1310). In this step the alcohol hydroxy is selectively protected using a suitable protecting agent, such as acetic anhydride for example, in the presence of a base, such as pyridine for example. The carboxylic acid of Formula XXXIV is then activated by a coupling reagent, such as BOP, HATU, EEDQ, or CDI for example, and reacted with an amine of Formula XXXV, prepared according to Schemes 2b or 2c, to form a compound of Formula XXXVI. The amino moiety of Formula XXXVI is then selectively deprotected using a variety of methods known to those of skill in the art including the use of iodotrimethylsilane (TMS-I) in dichloromethane. Deprotection of the amine is followed by reaction with an isocyanate having A, such as 4-chlorophenyl isocyanate for example, in the presence of a suitable solvent, such as THF for example, and a base, such as triethylamine for example, to afford the compound of Formula XXXVII. The alcohol hydroxy of a compound of FormulaXXXVII is then selectively deprotected using a variety of methods known to those of skill in the art including the use of potassium trimethylsilanolate in a THF to afford a compound of Formula I.

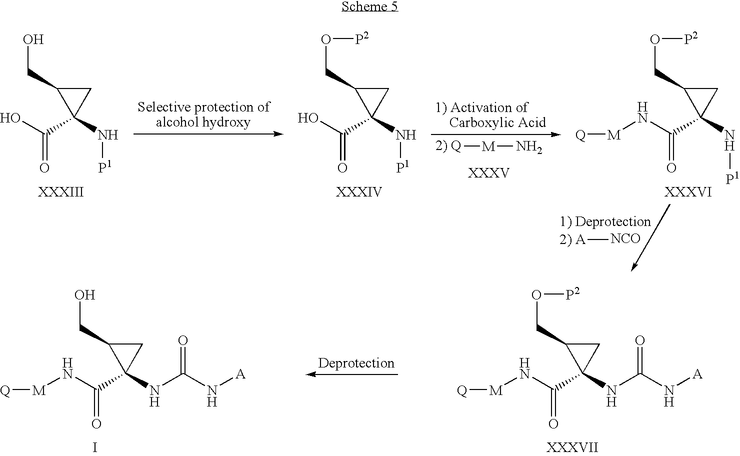

Another method of making the compounds having Formula I is provided in Scheme 6 where P¹, P² and P³ are independent protecting groups. Scheme 6 is useful for synthesizing compounds of Formula I where X¹ and X² form a substituted nitrogen containing heterocyclic ring such as a pyrrolidine ring substituted at the 1 position for example. The synthetic route starts with reaction of a compound of Formula XXXVIII (prepared according to the methods disclosed in C. Balsamini, E. Duranti, L. Mariani, A. Salvatori, G. Spadoni. Synthesis 1990, 779-781, and O. Mamoun, H. Benhaoua, R. Danion-Bougot, D. Danion. Synth. Comm., 1995, 25, 1295), where P² is any suitable carboxylic acid protecting group such as that which would form an alkyl ester, with acid in a suitable solvent to form a compound of Formula XXXIX. Suitable acids and solvents for the preparation of compounds of Formula XXXIX include mineral acids, such as hydrochloric acid for example, and nonpolar solvents, such as diethyl ether for example. The amino group of compounds of Formula XXXIX is then reacted with a reagent capable of forming a protecting group, P¹, on the amino moiety in a suitable solvent to form a compound with Formula XL. Useful reagents for protecting an amino acid include di-tert-butyl dicarbonate. The heterocycle nitrogen is then reacted with a reagent capable of forming a protecting group, P³, in a suitable solvent to form a compound with Formula XLI. Useful reagents for protecting the heterocycle nitrogen include carbobenzyloxychloride for example. The compound of Formula XLI is then reacted with a base to selectively deprotect the carboxylic acid moiety to afford a free acid of Formula XLII. The reaction can be carried out using hydroxides of the alkaline earth metals such as lithium hydroxide, for example in a suitable nonpolar solvent such as tetrahydrofuran, for example. The carboxylic acid of Formula XLII is then activated by a coupling reagent, such as BOP, HATU, EEDQ, or CDI for example, and reacted with an amine of Formula XLIII, prepared according to Schemes 2b or 2c, to form a compound of Formula XLIV. The compound of Formula XLIV is then selectively deprotected using standard methods of amine deprotection, such as TFA in chloroform for example, to afford the free amine of Formula XLV. The compound of Formula XLV is then reacted with an isocyanate having A, such as 4-chlorophenyl isocyanate for example, in the presence of a suitable solvent, such as THF for example, and a base, such as triethylamine for example, to afford a compound of Formula I. The protecting group for the heterocycle nitrogen may be removed using standard methods for deprotection of an amine which are known to those of skill in the art or the nitrogen may remain protected.

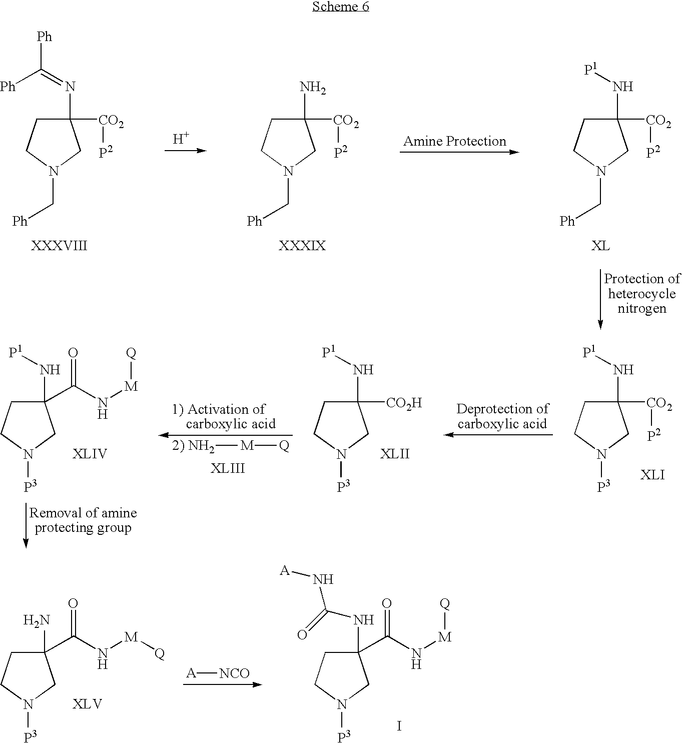

Not all compounds of Formula I falling into a given class may be compatible with some of the reaction conditions described. Such restrictions are readily apparent to those skilled in the art of organic synthesis, and alternative methods must then be used.

To further assist in understanding the present invention, the following non-limiting examples of such factor Xa inhibitory compounds are provided. The following examples, of course, should not be construed as specifically limiting the present invention, variations presently known or later developed, which would be within the purview of one skilled in the art and considered to fall within the scope of the present invention as described herein. Preferred synthetic routes for intermediates involved in the synthesis as well as the resulting anti-thrombotic compounds of the present invention follow.

### EXAMPLES

### Example 1

### 1-[3-(4-Chloro-phenyl)-ureido]-cyclopentanecarboxylic acid (2′-methanesulfonyl-biphenyl-4-yl)-amide (compound 1)

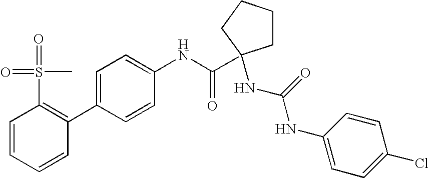

Step 1: [1-(4-Bromo-phenylcarbamoyl)-cyclopentyl]-carbamic acid tert-butyl ester (1a). 1-tert-Butoxycarbonylamino-cyclopentanecarboxylic acid (0.500 g, 2.18 mmol), 4-bromoaniline (0.37 g, 2.18 mmol) and EEDQ (0.646 g, 2.60 mmol) were dissolved in dry CHCl₃ (20 mL). Triethylamine (0.445 mL, 3.27 mmol) was added, and the solution heated at reflux for 19 h. The reaction was allowed to cool and then concentrated to a white solid which was partitioned between EtOAc and water. The aqueous layer was then extracted with EtOAc/THF (3:1), and the organic layers combined, washed with brine and dried over MgSO₄. Concentration of the organic layer, and purification of the crude product by crystallization (THF/Hexane) revealed 1a (0.463 g, 55%):

Step 2: [1-(2′-Methylsulfanyl-biphenyl-4-ylcarbamoyl)-cyclopentyl]-carbamic acid tert-butyl ester (1b). A mixture of 1a (0.25 g, 0.652 mmol), 2-(methylthio)benzeneboronic acid (0.131 g, 0.782 mmol), tetrabutylammonium bromide (0.011 g, 0.033 mmol), sodium carbonate (0.138 g, 1.304 mmol), and water (1 mL) in toluene (6 mL) was degassed with a stream of argon. Tetrakis(triphenylphosphine) Pd(0) (0.038 g, 0.0326 mmol) was then added, and the mixture heated at reflux under an argon atmosphere for 22 h. The resulting solution was allowed to cool to RT and concentrated to a solid which was partitioned between EtOAc and water. The organic layer was separated and washed with brine and dried over MgSO₄. Concentration of the organic layer, and purification of the resulting residue by MPLC resulted in the product 1b (0.160 g, 57%) as a light yellow solid.

Step 3: [1-(2′-Methanesulfonyl-biphenyl-4-ylcarbamoyl)-cyclopentyl]-carbamic acid tert-butyl ester (1c). To a mixture of 1b (0.150 g, 0.352 mmol) in EtOAc (35 mL) was added m-CPBA (70%, 0.347 g, 1.40 mmol) to form a solution. The solution was allowed to stir 4.5 h and then washed sequentially with 10% aq. Na₂S₂O₃, sat. aq. NaHCO₃, water and brine before being dried over MgSO₄. Concentration of the solution under reduced pressure and purification of the crude product by MPLC revealed 1c (0.128 g, 79%) as light yellow solid.

Step 4: 1-[3-(4-Chloro-phenyl)-ureido]-cyclopentanecarboxylic acid (2′-methanesulfonyl-biphenyl-4-yl)-amide (1). Into a solution of 1c (0.125 g, 0.272 mmol) in dry DCM (4 mL) was added TFA (2 mL), and the solution stirred at RT for 2 h. The solution was then concentrated under reduced pressure and dried under vacuum. The crude product was dissolved in dry THF (4 mL) and cooled to 0° C. in an ice bath. Triethylamine (0.190 mL, 1.36 mmol) was then added followed by 4-chlorophenyl isocyanate (0.042 g, 0.272 mmol). The reaction was allowed to stir at RT for 1.5 h before being concentrated under reduced pressure, and the resulting crude product purified by MPLC. The product isolated was further purified by recrystallization from THF/hexanes to yield 1 (0.115 g, 82%) as a white solid: MS: APCI (AP+): 512 (M)+; CHN calc'd for C₂₆H₂₆Cl₁N₃O₄S₁: % C 60.99; % H 5.12; % N 8.21. Found: % C 60.94; % H 5.22; % N 7.89.

### Example 2

### 1-[3-(4-Chloro-phenyl)-ureido]-cyclopropanecarboxylic acid (2′-methanesulfonyl-biphenyl-4-yl)-amide (compound 2)

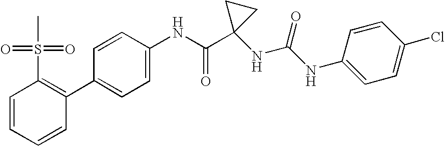

Step 1: [1-(4-Bromo-phenylcarbamoyl)-cyclopropyl]-carbamic acid tert-butyl ester (2a). 1-tert-Butoxycarbonylamino-cyclopropanecarboxylic acid (0.50 g, 2.49 mmol), 4-bromoaniline (0.423 g, 2.49 mmol) and EEDQ (0.735 g, 2.99 mmol) were dissolved in dry CHCl₃ (20 mL). Triethylamine (0.507 mL, 3.73 mmol) was added, and the solution heated at reflux for 18 h. The reaction was allowed to cool and then concentrated to a white solid which was partitioned between EtOAc and water. The aqueous layer was then extracted with EtOAc again, and the organic layers combined, washed with brine and dried over MgSO₄. Concentration of the organic layer, and purification of the crude product by recrystallization (EtOAc/Hex) revealed 2a (0.574 g, 82%)

Step 2: [1-(2′-Methylsulfanyl-biphenyl-4-ylcarbamoyl)-cyclopropyl]-carbamic acid tert-butyl ester (2b). A mixture of 2a (0.680 g, 1.91 mmol), 2-(methylthio)benzene boronic acid (0.385 g, 2.29 mmol), tetrabutylammonium bromide (0.031 g, 0.096 mmol), sodium carbonate (0.405 g, 3.82 mmol), and water (2 mL) in toluene (20 mL) was degassed with a stream of argon. Tetrakis(triphenylphosphine) Pd(0) (0.220 g, 0.191 mmol) was added, and the mixture heated at reflux under an argon atmosphere for 1.5 h. The resulting solution was allowed to cool to RT and concentrated to a solid which was partitioned between EtOAc and water. The organic layer was separated and washed with brine and dried over MgSO₄. Concentration of the organic layer, and purification of the resulting residue by MPLC resulted in the product 2b (0.630 g, 83%) as a yellow solid.

Step 3: [1-(2′-Methanesulfonyl-biphenyl-4-ylcarbamoyl)-cyclopropyl]-carbamic acid tert-butyl ester (2c). To a mixture of 2b (0.600 g, 1.50 mmol) in EtOAc (15 mL) was added m-CPBA (70%, 1.04 g, 6.02 mmol) to form a solution which was stirred 2 h. EtOAc (15 mL) was added, and the reaction was washed sequentially with 10% aq. Na₂S₂O₃, sat. aq. NaHCO₃, water and brine before being dried over MgSO₄. Concentration of the solution under reduced pressure and purification of the crude product by recrystallization from EtOAc/Hexanes revealed 2c (0.475 g, 73%) as white solid.

Step 4: 1-[3-(4-Chloro-phenyl)-ureido]-cyclopropanecarboxylic acid (2′-methanesulfonyl-biphenyl-4-yl)-amide (2). Into a solution of 2c (0.30 g, 0.696 mmol) in dry DCM (8 mL) was added TFA (2 mL), and the solution stirred at RT for 1.5 h. The solution was then concentrated under reduced pressure and dried under vacuum. The crude product was dissolved in dry THF (8 mL) and cooled to 0° C. in an ice bath. Triethylamine (0.485 mL, 3.48 mmol) was added followed by the addition of 4-chlorophenyl isocyanate (0.107 g, 0.696 mmol). The reaction was allowed to stir at RT for 1 h before being concentrated under reduced pressure, and the resulting crude product purified by MPLC. The product isolated was further purified by recrystallization from EtOAc/hexanes to yield 2 (0.270 g, 80%) as a white solid: MS: APCI (AP+): 484 (M+H)⁺; CHN calc'd for C₂₄H₂₂Cl₁N₃O₄S₁+0.42H₂O: calc'd.: % C 58.64; % H 4.68; % N 8.55. Found: % C 58.25; % H 4.71; % N 8.34.

### Example 3

### 1-[3-(4-Chloro-phenyl)-ureido]-cyclopropanecarboxylic acid (3-fluoro-2′-methanesulfonyl-biphenyl-4-yl)-amide (compound 3)

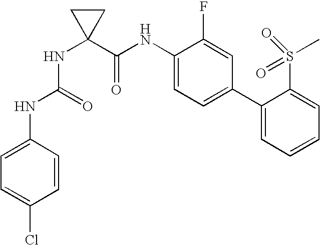

Step 1: [1-(3-Fluoro-2′-methylsulfanyl-biphenyl-4-ylcarbamoyl)-cyclopropyl]-carbamic acid tert-butyl ester (3a). 1-tert-Butoxycarbonylamino-cyclopropanecarboxylic acid (1.00 g, 4.97 mmol), 3-fluoro-2′-methylsulfanyl-biphenyl-4-ylamine (1.159 g, 4.97 mmol), and EEDQ (1.475 g, 5.96 mmol) were dissolved in dry CHCl₃ (20 mL). Triethylamine (1.039 mL, 7.45 mmol) was added and the solution heated at reflux for 20 h. The reaction was allowed to cool and EtOAc was added. The solution was washed sequentially with 10% aq. citric acid, 1N NaOH, water, and then brine before drying the solution over MgSO₄. Concentration of the solution under reduced pressure and purification of the crude product by flash chromatography revealed slightly impure 3a (2.10 g) as a white foam.

Step 2: [1-(3-Fluoro-2′-methanesulfonyl-biphenyl-4-ylcarbamoyl)-cyclopropyl]-carbamic acid tert-butyl ester (3b). To a mixture of 3a (1.80 g, 4.32 mmol) in EtOAc (43 mL) was added m-CPBA (70%, 4.26 g, 17.3 mmol). The solution was stirred for 2.5 h at RT before diluting with EtOAc. The solution was washed sequentially with sat. aq. Na₂S₂O₃, two portions of sat. aq. NaHCO₃, water and then brine before drying over MgSO₄. Concentration of the solution under reduced pressure and purification of the crude product by flash chromatography revealed 3b (1.72 g, 89%) as a white solid.

Step 3: 1-[3-(4-Chloro-phenyl)-ureido]-cyclopropanecarboxylic acid (3-fluoro-2′-methanesulfonyl-biphenyl-4-yl)-amide (3). Into a solution of 3b (0.231 g, 0.515 mmol) in dry DCM (5 mL) was added TFA (3 mL), and the solution stirred at RT for 1.5 h. The solution was then concentrated under reduced pressure and dried under vacuum. The crude product was dissolved in dry THF (10 mL) and cooled to 0° C. in an ice bath. Triethylamine (0.359 mL, 2.58 mmol) was then added followed by 4-chlorophenyl isocyanate (0.079 g, 0.515 mmol). The reaction was stirred at RT for 2 h before concentrating under reduced pressure. The resulting crude product was purified by flash chromatography to reveal 3 (0.162 g, 63%) as a white solid: MS: APCI (AP+): 502 (M)+; CHN calc'd for C₂₄H₂₁Cl₁F₁N₃O₄S₁: % C 57.36; % H 4.22; % N 8.36. Found: % C 56.97; % H 4.07; % N 8.05.

### Example 4

### 1-[3-(5-Chloro-pyridin-2-yl)-ureido]-cyclopropanecarboxylic acid (3-fluoro-2′-methanesulfonyl-biphenyl-4-yl)-amide (compound 4)

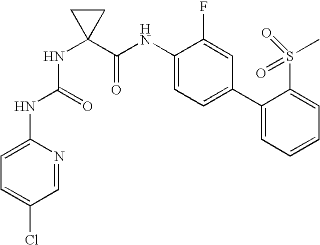

Step 1: (5-Chloro-pyridin-2-yl)-carbamic acid 4-nitro-phenyl ester (4a). 2-Amino-5-chloropyridine (2.10 g, 16.3 mmol) was suspended in dry DCM (30 mL). Pyridine (1.32 mL, 16.3 mmol) was added, and the mixture cooled to 0° C. in an ice bath. 4-Nitrophenyl chloroformate (1.32 mL, 16.3 mmol) was added causing a white precipitate to form. The reaction was stirred at RT for 1 h before water was added and the mixture filtered through a glass frit. The filtrate was washed with two portions of DCM and air dried overnight to give 4a (4.31 g, 90%) as a white solid.

Step 2: 1-[3-(4-Chloro-phenyl)-ureido]-cyclopropanecarboxylic acid (3-fluoro-2′-methanesulfonyl-biphenyl-4-yl)-amide (4b). Into a solution of 3b (1.72 g, 3.84 mmol) in dry DCM (10 mL) was added TFA (5 mL), and the solution stirred at RT for 1.5 h. The solution was then concentrated under reduced pressure and dried under vacuum. The crude product was dissolved in dry DMF (40 mL). Triethylamine (2.68 mL, 19.2 mmol) was added followed by 4a (1.13 g, 3.84 mmol). The reaction was stirred at 50° C. for 3 h before EtOAc was added and washed sequentially with four portions of sat. aq. NaHCO₃, and one portion each of 10% aq. citric acid and brine. The solution was dried over MgSO₄, concentrated under reduced pressure, and the resulting crude product purified by flash chromatography. Lyophilization from MeCN/H₂O revealed 4b (0.163 g, 8%) as a white solid: MS: APCI (AP+): 503 (M)+; CHN cac'd for C₂₃H₂₀Cl₁F₁N₄O₄S₁: % C 54.93; % H4.01; % N 11.14. Found: % C 54.68; % H 3.80; % N 10.98.

### Example 5

### 1-[3-(4-Chloro-phenyl)-ureido]-cyclopropanecarboxylic acid (3-fluoro-2′-sulfamoyl-biphenyl-4-yl)-amide (compound 5)

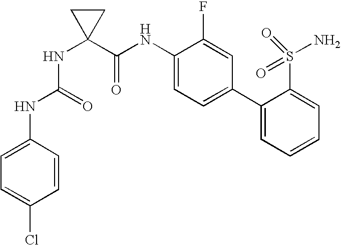

Step 1: [1-(2′-tert-Butylsulfamoyl-3-fluoro-biphenyl-4-ylcarbamoyl)-cyclopropyl]-carbamic acid tert-butyl ester (5a). 1-tert-Butoxycarbonylamino-cyclopropanecarboxylic acid (0.500 g, 2.49 mmol), 4′-amino-3′-fluoro-biphenyl-2-sulfonic acid tert-butylamide (0.801 g, 2.49 mmol), and EEDQ (0.737 g, 2.98 mmol) were dissolved in dry CHCl₃ (10 mL). Triethylamine (0.520 mL, 3.73 mmol) was added and the solution heated at reflux for 17 h. The reaction was allowed to cool and EtOAc was added. The solution was washed sequentially with 10% aq. citric acid, 1N NaOH, water, and then brine before drying over MgSO₄. Concentration of the solution under reduced pressure and purification of the crude product by flash chromatography revealed 5a (0.977 g, 78%) as a white foam.

Step 2: 1-Amino-cyclopropanecarboxylic acid (3-fluoro-2′-sulfamoyl-biphenyl-4-yl)-amide (5b). To 5a (0.972 g, 1.922 mmol) was added TFA (1 mL), and the solution stirred at reflux for 30 min. The solution was then concentrated under reduced pressure and dried under vacuum to yield 5b (0.67 g, 100%) as an oil.

Step 3: 1-[3-(4-Chloro-phenyl)-ureido]-cyclopropanecarboxylic acid (3-fluoro-2′-sulfamoyl-biphenyl-4-yl)-amide (5). Compound 5b (0.33 g, 0.961 mmol) was dissolved in dry THF (18 mL) and cooled to 0° C. in an ice bath. Triethylamine (1.19 mL, 8.55 mmol) was then added followed by 4-chlorophenyl isocyanate (0.263 g, 1.71 mmol). The reaction was allowed to stir at RT for 2 h before concentrating under reduced pressure. The resulting crude product was purified by flash chromatography followed by reverse phase preparatory HPLC to reveal 5 (0.154 g, 32%) as a white solid: MS: APCI (AP+): 503 (M)+; CHN calc'd for C₂₃H₂₀Cl₁F₁N₄O₄S₁: % C 51.13; % H 3.81; % N 9.98. Found: % C 50.75; % H 3.58; % N 9.76.

### Example 6

### 1-[3-(5-Chloro-pyridin-2-yl)-ureido]-cyclopropanecarboxylic acid (3-fluoro-2′-sulfamoyl-biphenyl-4-yl)-amide (compound 6)

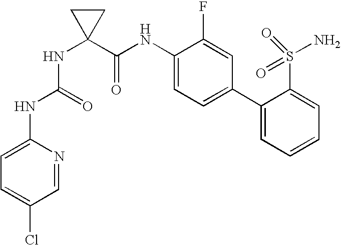

Step 1: 1-[3-(5-Chloro-pyridin-2-yl)-ureido]-cyclopropanecarboxylic acid (3-fluoro-2′-sulfamoyl-biphenyl-4-yl)-amide (6). To a solution of 5b (0.33 g, 0.961 mmol) in dry DMF (20 mL) was added triethylamine (0.885 mL, 6.35 mmol) followed by 4a (0.373 g, 1.27 mmol). The reaction was stirred at 50° C. for 2 h before cooling, adding EtOAc, and washing sequentially with four portions of sat. aq. NaHCO₃, and one portion each of 10% aq. citric acid and brine. The solution was dried over MgSO₄ and concentrated under reduced pressure. Purification of the crude product by flash chromatography followed by reverse phase preparatory HPLC revealed 6 (0.244 g, 50%) as a white solid. MS: APCI (AP+): 504 (M)+, (AP−): 502 (M)−; CHN calc'd for C₂₂H₁₉Cl₁F₁N₅O₄S₁: % C 45.60; % H 3.32; % N 10.99. Found: % C 45.21; % H 3.21; % N 10.76.

### Example 7

### 2-[3-(4-Chloro-phenyl)-ureido]-N-(3-fluoro-2′-methanesulfonyl-biphenyl-4-yl)-2-methyl-propionamide (compound 7)

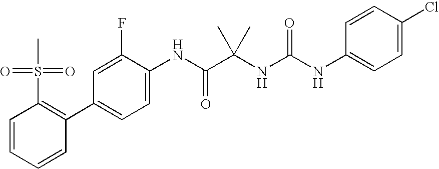

Step 1: [1-(3-Fluoro-2′-methylsulfanyl-biphenyl-4-ylcarbamoyl)-1-methyl-ethyl]-carbamic acid tert-butyl ester (7a). 2-tert-Butoxycarbonylamino-2-methyl-propionic acid (0.50 g, 2.46 mmol), 3-fluoro-2′-methylsulfanyl-biphenyl-4-ylamine (0.573 g, 2.46 mmol) and EEDQ (0.729 g, 2.95 mmol) were dissolved in dry CHCl₃ (25 mL). Triethylamine (0.514 mL, 3.69 mmol) was added, and the solution heated at reflux for 40 h. The reaction was allowed to cool and then concentrated to a white solid which was partitioned between EtOAc and water. The aqueous layer was then extracted with EtOAc again, and the organic layers combined, washed with brine and dried over MgSO₄. Concentration of the organic layer and purification of the crude product by MPLC revealed compound 7a (0.337 g, 33%) as a white solid.

Step 2: [1-(3-Fluoro-2′-methanesulfonyl-biphenyl-4-ylcarbamoyl)-1-methyl-ethyl]-carbamic acid tert-butyl ester (7b). To a mixture of compound 7a (0.300 g, 0.71 mmol) in EtOAc (8 mL) was added m-CPBA (70%, 0.708 g, 2.86 mmol) to form a solution which was stirred for 2.5 h. EtOAc (15 mL) was added, and the reaction was washed sequentially with 10% aq. Na₂S₂O₃, sat. aq. NaHCO₃, water and brine before being dried over MgSO₄. Concentration of the solution under reduced pressure revealed the product as an oil. Hexanes were added, and the product concentrated under reduced pressure to reveal compound 7b (0.317 g, 99%) as a white foam that solidified under vacuum. The solid was of sufficient purity as to use in subsequent reactions.

Step 3: 2-[3-(4-Chloro-phenyl)-ureido]-N-(3-fluoro-2′-methanesulfonyl-biphenyl-4-yl)-2-methyl-propionamide (7). Into a solution of compound 7b (0.30 g, 0.696 mmol) in dry DCM (8 mL) was added TFA (2 mL), and the solution stirred at RT for 1.5 h. The solution was then concentrated under reduced pressure and dried under vacuum. The crude product was dissolved in dry THF (8 mL) and cooled to 0° C. in an ice bath. Triethylamine (0.485 mL, 3.48 mmol) was added followed by the addition of 4-chlorophenyl isocyanate (0.107 g, 0.696 mmol). The reaction was allowed to stir at RT for 1 h before being concentrated under reduced pressure, and the resulting crude product purified by MPLC. The product isolated was further purified by recrystallization from EtOAc/hexanes to yield compound 7 (0.270 g, 80%) as a white solid: MS: APCI (AP+): 484 (M+H)⁺; CHN calc'd for C₂₄H₂₂Cl₁N₃O₄S₁+0.42H₂O: calc'd.: % C 58.64; % H 4.68; % N 8.55. Found: % C 58.25; % H 4.71; % N 8.34.

### Example 8

### 2-[3-(5-Chloro-pyridin-2-yl)-ureido]-N-(3-fluoro-2′-methanesulfonyl-biphenyl-4-yl)-2-methyl-propionamide (compound 8)

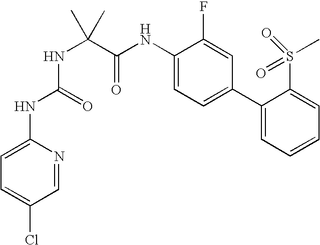

Step 1: [1-(3-Fluoro-2′-methylsulfanyl-biphenyl-4-ylcarbamoyl)-1-methyl-ethyl]-carbamic acid tert-butyl ester (8a). 2-tert-Butoxycarbonylamino-2-methyl-propionic acid (0.500 g, 2.46 mmol), 3-fluoro-2′-methylsulfanyl-biphenyl-4-ylamine (0.574 g, 2.46 mmol), and EEDQ (0.730 g, 2.95 mmol) were dissolved in dry CHCl₃ (10 mL). Triethylamine (0.514 mL, 3.69 mmol) was added and the solution heated at reflux for 20 h before cooling and adding EtOAc. The solution was washed sequentially with 10% aq. citric acid, 1N NaOH, water, and then brine before drying the solution over MgSO₄. Concentration of the solution under reduced pressure and purification of the crude product by flash chromatography revealed compound 8a (0.427 g, 41%) as a white foam.

Step 2: [1-(3-Fluoro-2′-methanesulfonyl-biphenyl-4-ylcarbamoyl)-1-methyl-ethyl]-carbamic acid tert-butyl ester (8b). To a mixture of compound 8a (0.427 g, 1.02 mmol) in EtOAc (10 mL) was added m-CPBA (70%, 1.01 g, 4.08 mmol). The solution was allowed to stir for 2 h at RT before diluting with EtOAc. The solution was washed sequentially with sat. aq. Na₂S₂O₃, sat. aq. NaHCO₃ twice, water, and then brine before drying over MgSO₄. Concentration of the solution under reduced pressure and purification of the crude product by flash chromatography revealed compound 8b (0.386 g, 84%) as a white solid.

Step 3: 2-[3-(5-Chloro-pyridin-2-yl)-ureido]-N-(3-fluoro-2′-methanesulfonyl-biphenyl-4-yl)-2-methyl-propionamide (8). Into a solution of compound 8b (0.382 g, 0.848 mmol) in dry DCM (5 mL) was added TFA (2 mL), and the solution stirred at RT for 1 h. The solution was then concentrated under reduced pressure and dried under vacuum. The crude product (0.297 g, 0.848 mmol) was dissolved in dry DMF (13 mL). Triethylamine (0.591 mL, 4.24 mmol) was added followed by 4a (0.249 g, 0.848 mmol). The reaction was stirred at 50° C. for 1 h before cooling, adding EtOAc, and washing sequentially with five portions of sat. aq. NaHCO₃, and one portion each of 10% aq. citric acid and brine. The solution was dried over MgSO₄ and concentrated under reduced pressure. Purification of the crude product by flash chromatography revealed compound 8 (0.109 g, 25%) as a white solid. MS: APCI (AP+): 505 (M)+; CHN calc'd for C₂₃H₂₂Cl₁F₁N₄O₄S₁: % C 54.24; % H 4.36; % N 10.95. Found: % C 54.25; % H 4.11; % N 11.14.

### Example 9

### 2-[3-(4-Chloro-phenyl)-ureidol-N-(3-fluoro-2′-sulfamoyl-biphenyl-4-yl)-2-methyl-propionamide (compound 9)

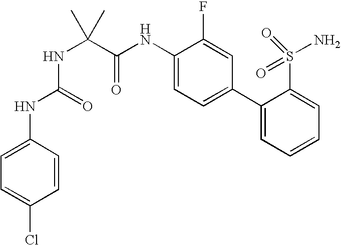

Step 1: [1-(2′-tert-Butylsulfamoyl-3-fluoro-biphenyl-4-ylcarbamoyl)-1-methyl-ethyl]-carbamic acid tert-butyl ester (9a). 2-tert-Butoxycarbonylamino-2-methyl-propionic acid (1.00 g, 4.92 mmol), 4′-amino-3′-fluoro-biphenyl-2-sulfonic acid tert-butylamide (1.59 g, 4.92 mmol), and EEDQ (1.46 g, 5.90 mmol) were dissolved in dry CHCl₃ (20 mL). Triethylamine (1.03 mL, 7.38 mmol) was added and the solution heated at reflux for 17 h. The reaction was allowed to cool to RT and EtOAc added. The solution was washed sequentially with 10% aq. citric acid, 1N NaOH, water, and then brine before drying over MgSO₄. Concentration of the solution under reduced pressure and purification of the crude product by flash chromatography revealed 9a (2.32 g, 62%).

Step 2: 2-Amino-N-(3-fluoro-2′-sulfamoyl-biphenyl-4-yl)-2-methyl-propionamide (9b). A solution of 9a (2.32 g, 2.84 mmol) and TFA (5 mL) was stirred at reflux for 2 h. The solution was then concentrated under reduced pressure and dried under vacuum to yield 9b (0.997 g, 100%) as an oil.

Step 3: 2-[3-(4-Chloro-phenyl)-ureido]-N-(3-fluoro-2′-sulfamoyl-biphenyl-4-yl)-2-methyl-propionamide (9). Compound 9b (1.16 g, 1.42 mmol) was dissolved in dry THF (15 mL) and cooled to 0° C. in an ice bath. Triethylamine (0.989 mL, 7.09 mmol) was then added followed by 4-chlorophenyl isocyanate (0.218 g, 1.42 mmol). The reaction was allowed to stir at RT for 1.5 h before concentrating under reduced pressure. The resulting crude product was purified by flash chromatography followed by reverse phase preparatory HPLC to yield compound 9 (0.169 g, 24%) as a white solid: MS: APCI (AP+): 505 (M)+; CHN calc'd for C₂₃H₂₂Cl₁F₁N₄O₄S₁: % C 52.31; % H 4.20; % N 10.34. Found: % C 52.10; % H 4.09; % N 10.25.

### Example 10

### 1-[3-(4-Chloro-phenyl)-ureido]-cyclohexanecarboxylic acid (2′-methanesulfonyl-biphenyl-4-yl)-amide (compound 10)

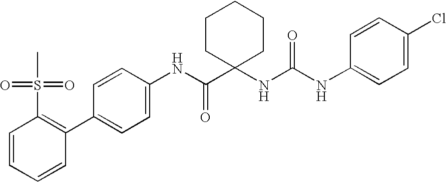

Step 1: [1-(4-Bromo-phenylcarbamoyl)-cyclohexyl]-carbamic acid tert-butyl ester (10a). 1-tert-Butoxycarbonylamino-cyclohexanecarboxylic acid (1.00 g, 4.11 mmol), 4-bromoaniline (0.699 g, 4.11 mmol) and EEDQ (0.841 g, 4.93 mmol) were dissolved in dry CHCl₃ (40 mL). Triethylamine (0.841 mL, 6.16 mmol) was added, and the solution heated at reflux for 53 h. The reaction was allowed to cool to RT and then concentrated to a white solid which was partitioned between EtOAc and water. The aqueous layer was then extracted with EtOAc again, and the organic layers combined, washed with brine and dried over MgSO₄. Concentration of the organic layer and purification of the crude product by recrystallization (EtOAc/Hex) revealed 10a (1.01 g, 62%) as a white solid.

Step 2: [1-(2′-Methylsulfanyl-biphenyl-4-ylcarbamoyl)-cyclohexyl]-carbamic acid tert-butyl ester (10b). A mixture of 10a (0.80 g, 2.01 mmol), 2-(methylthio)benzeneboronic acid (0.405 g, 2.41 mmol), tetrabutylammonium bromide (0.032 g, 0.10 mmol), sodium carbonate (0.426 g, 4.02 mmol), and water (3 mL) in toluene (22 mL) was degassed with a stream of argon. Tetrakis(triphenylphosphine) Pd(0) (0.232 g, 0.201 mmol) was added, and the mixture heated at reflux under an argon atmosphere for 2 h. The resulting solution was allowed to cool to RT and concentrated to a solid that was partitioned between EtOAc and water. The organic layer was separated and washed with brine and dried over MgSO₄. Concentration of the organic layer, and purification of the resulting residue by MPLC resulted in 10b (0.610 g, 66%) as a light yellow solid.

Step 3: [1-(2′-Methanesulfonyl-biphenyl-4-ylcarbamoyl)-cyclohexyl]-carbamic acid tert-butyl ester (10c). To a mixture of 10b (0.590 g, 1.28 mmol) in EtOAc (20 mL) was added m-CPBA (70%, 1.27 g, 5.14 mmol) and DCM (15 mL) to form a solution which was stirred for 2 h. EtOAc (25 mL) was added, and then the reaction was washed sequentially with 10% aq. Na₂S₂O₃, sat. aq. NaHCO₃, water and brine before being dried over MgSO₄. Concentration of this solution under reduced pressure revealed a solid which was recrystalized from THF/Hexanes to reveal compound 10c (0.390 g, 65%) as a white solid.

Step 4: 1-[3-(4-Chloro-phenyl)-ureido]-cyclohexanecarboxylic acid (2′-methanesulfonyl-biphenyl-4-yl)-amide (10). Into a solution of 10c (0.365 g, 0.77 mmol) in dry DCM (8 mL) was added TFA (2 mL), and the solution stirred at RT for 1.5 h. The solution was then concentrated under reduced pressure. Hexanes were added, and the residue concentrated again under reduced pressure. The resulting oil was then dried under vacuum. The crude product was dissolved in dry THF (10 mL) and cooled to 0° C. in an ice bath. Triethylamine (0.430 mL, 3.08 mmol) was added followed by the addition of 4-chlorophenyl isocyanate (0.118 g, 0.77 mmol). The reaction was allowed to stir at RT for 1 h before being concentrated under reduced pressure and the resulting crude product purified by MPLC to yield a white solid which was further purified by recrystalization from EtOAc/Hex to reveal compound 10 (0.142 g, 35%) as a white solid: MS: APCI (AP−): 526 (M−H)⁺; CHN calc'd for C₂₇H₂₈Cl₁N₃O₄S₁: calc'd.: % C 61.65; % H 5.37; % N 7.99. Found: % C 61.53; % H 5.30; % N 7.90.

### Example 11

### 1-[3-(4-Chloro-phenyl)-ureido]-cyclopent-3-enecarboxylic acid (3-fluoro-2′-sulfamoyl-biphenyl-4-yl)-amide (compound 11)

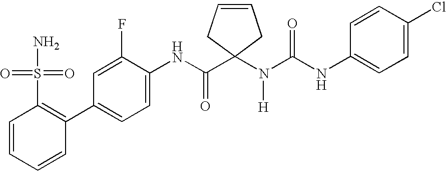

Step 1: [1-(2′-tert-Butylsulfamoyl-3-fluoro-biphenyl-4-ylcarbamoyl)-cyclopent-3-enyl]-carbamic acid tert-butyl ester (11a). 1-tert-Butoxycarbonylamino-cyclopent-3-enecarboxylic acid (0.500 g, 2.20 mmol), 4′-amino-3′-fluoro-biphenyl-2-sulfonic acid tert-butylamide (0.709 g, 2.20 mmol) and EEDQ (0.652 g, 2.64 mmol) were dissolved in dry CHCl₃ (22 mL). Triethylamine (0.460 mL, 3.30 mmol) was added, and the solution heated at reflux for 40 h. The reaction was allowed to cool to RT and then concentrated to a white solid which was partitioned between EtOAc and water. The aqueous layer was then extracted with EtOAc again, and the organic layers combined, washed with brine and dried over MgSO₄. Concentration of the organic layer and purification of the crude product by MPLC revealed a mixture of 11a (0.800 g) contaminated with 4′-amino-3′-fluoro-biphenyl-2-sulfonic acid tert-butylamide. The impure product was used in the subsequent reaction.

Step 2: 1-[3-(4-Chloro-phenyl)-ureido]-cyclopent-3-enecarboxylic acid (2′-tert-butylsulfamoyl-3-fluoro-biphenyl-4-yl)-amide (11b). Into a solution of 11a (0.320 g, 0.587 mmol) in dry DCM (6 mL) was added TFA (2 mL), and the solution stirred at RT for 1 h. The solution was then concentrated under reduced pressure. Chloroform was added, and the residue concentrated again under reduced pressure. The resulting oil was then dried under vacuum. The crude product was dissolved in dry THF (8 mL) and cooled to 0° C. in an ice bath. Triethylamine (0.728 mL, 5.21 mmol) was then added followed by the portion-wise addition of 4-chlorophenyl isocyanate (0.271 g, 1.76 mmol). After 5 h, the reaction was concentrated under reduced pressure, and the resulting crude product purified by MPLC to reveal 11b (0.289 g, 84%) as a white solid:

Step 3: 1-[3-(4-Chloro-phenyl)-ureido]-cyclopent-3-enecarboxylic acid (3-fluoro-2′-sulfamoyl-biphenyl-4-yl)-amide (11). A solution of 11b (0.269 g, 0.46 mmol) and TFA (5 mL) was stirred for 6 h before being concentrated under reduced pressure. The resulting oil was dissolved in CHCl₃ and concentrated again under reduced pressure, and the resulting residue purified by MPLC to reveal the product 11 (0.110 g, 45%) as a white solid: MS: APCI (AP+): 529 (M−H)⁺; CHN calc'd for C₂₅H₂₂Cl₁F₁N₄O₄S₁: calc'd.: % C 56.76; % H 4.19; % N 10.59. Found: % C 56.71; % H 4.13; % N 10.21.

### Example 12

### 2-[3-(4-Chloro-phenyl)-1-methyl-ureido]-N-(3-fluoro-2′-methanesulfonyl-biphenyl-4-yl)-acetamide (compound 12)

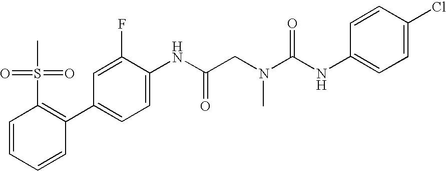

Step 1: [(3-Fluoro-2′-methylsulfanyl-biphenyl-4-ylcarbamoyl)-methyl]-methyl-carbamic acid tert-butyl ester (12a). (tert-Butoxycarbonyl-methyl-amino)-acetic acid (1.0 g, 5.28 mmol), 3-fluoro-2′-methylsulfanyl-biphenyl-4-ylamine (1.23 g, 5.28 mmol) and EEDQ (1.56 g, 6.33 mmol) were dissolved in dry CHCl₃ (40 mL). Triethylamine (1.10 mL, 7.92 mmol) was added, and the solution heated at reflux for 23.5 h. The reaction was allowed to cool and then concentrated to a solid which was partitioned between EtOAc and water. The aqueous layer was extracted with EtOAc again, and the organic layers combined, washed with brine and dried over MgSO₄. Concentration of the organic layer and purification of the crude product by MPLC revealed 12a (2.56 g) as an impure solid that was used in subsequent reactions.

Step 2: [(3-Fluoro-2′-methanesulfonyl-biphenyl-4-ylcarbamoyl)-methyl]-methyl-carbamic acid tert-butyl ester (12b). To a mixture of 12a (2.56 g, 6.32 mmol) in EtOAc (60 mL) was added m-CPBA (70%, 4.37 g, 25.32 mmol) to form a solution which was stirred for 2 h. EtOAc (25 mL) was added, and the reaction was washed sequentially with 10% aq. Na₂S₂O₃, sat. aq. NaHCO₃, water and brine before being dried over MgSO₄. Concentration of this solution under reduced pressure and purification of the resulting residue by MPLC revealed 12b (1.87 g, 68%) as an oil which was made solid by the addition of hexanes and subsequent concentration under reduced pressure followed by drying under vacuum.

Step 3: 2-[3-(4-Chloro-phenyl)-1-methyl-ureido]-N-(3-fluoro-2′-methanesulfonyl-biphenyl-4-yl)-acetamide (12). Into a solution 12b (0.300 g, 0.687 mmol) in dry DCM (5 mL) was added TFA (1 mL), and the solution stirred at RT for 1 h. The solution was then concentrated under reduced pressure. Chloroform was added, and the residue concentrated again under reduced pressure. The resulting oil was dried under vacuum. The crude product was dissolved in dry THF (6 mL) and cooled to 0° C. in an ice bath. Triethylamine (0.384 mL, 2.75 mmol) was then added followed by the addition of 4-chlorophenyl isocyanate (0.105 g, 0.687 mmol). The reaction was allowed to stir at RT for 2 h before being concentrated under reduced pressure, and the resulting crude product purified by MPLC to yield 12 (0.261 g, 77%) as a white solid: MS: APCI (AP+): 490 (M+H)⁺; CHN calc'd for C₂₃H₂₁Cl₁F₁N₃O₄S₁+0.03H₂O: calc'd.: % C 56.32; % H 4.33; % N 8.57; % H₂O 0.11. Found: % C 56.11; % H 4.27; % N 8.31, % H₂O 0.47.

### Example 13

### 2-[3-(4-Chloro-phenyl)-3-methyl-ureido]-N-(3-fluoro-2′-methanesulfonyl-biphenyl-4-yl)-acetamide (compound 13)

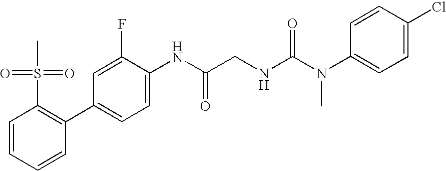

Step 1: 2-[3-(4-Chloro-phenyl)-3-methyl-ureido]-N-(3-fluoro-2′-methanesulfonyl-biphenyl-4-yl)-acetamide (13). Into a solution of 2-amino-N-(3-fluoro-2′-methanesulfonyl-biphenyl-4-yl)-acetamide (0.30 g, 0.710 mmol) in dry DCM (4 mL) was added TFA (1 mL), and the solution stirred at RT for 1 h. The solution was then concentrated under reduced pressure. Chloroform was added, and the residue concentrated again under reduced pressure. The resulting oil was dried under vacuum. The crude product was dissolved in dry THF (8 mL) and cooled to 0° C. in an ice bath. Triethylamine (0.396 mL, 2.84 mmol) was then added followed by 4-chloropheny-2-animochloroformate (0.145 g, 0.710 mmol) and catalytic DMAP (0.01 g). The reaction was allowed to stir at RT for 2.5 h before being concentrated under reduced pressure, and the resulting crude product partitioned between EtOAc and water. The organic layer was then washed with brine, dried over MgSO₄ and purified by MPLC to yield the product as a white solid which was recrystalized from EtOAc/Hexanes to give pure 13 (0.194 g, 55%): MS: APCI (AP+): 490 (M+H)⁺; CHN calc'd for C₂₃H₂₁Cl₁F₁N₃O₄S₁+0.05H₂O: calc'd.: % C 56.28; % H 4.33; % N 8.56. Found: % C 55.99; % H 4.14; % N 8.31.

### Example 14

### 2-[3-(4-Chloro-phenyl)-1,3-dimethyl-ureido]-N-(3-fluoro-2′-methanesulfonyl-biphenyl-4-yl)-acetamide (compound 14)

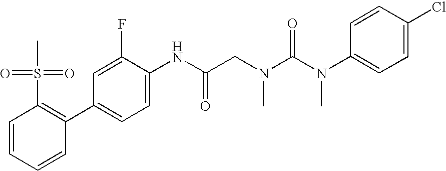

Step 1: 2-[3-(4-Chloro-phenyl)-1,3-dimethyl-ureido]-N-(3-fluoro-2′-methane-sulfonyl-biphenyl-4-yl)-acetamide (14). Into a solution of 12b (0.20 g, 0.458 mmol) in dry DCM (4 mL) was added TFA (1 mL), and the solution stirred at RT for 1 h. The solution was then concentrated under reduced pressure. Chloroform was added, and the residue concentrated again under reduced pressure. The resulting oil was then dried under vacuum. The crude product was dissolved in dry THF (4 mL) and cooled to 0° C. in an ice bath. Triethylamine (0.255 mL, 1.83 mmol) was then added followed by the addition of 4-chlorophenyl-2-animochloroformate (0.093 g, 0.458 mmol) and catalytic DMAP (0.01 g). The reaction was allowed to stir at RT for 18 h before being concentrated under reduced pressure, and the resulting crude product partitioned between EtOAc and water. The organic layer was then washed with brine, dried over MgSO₄ and purified by MPLC to yield the product as an oily foam. The product was concentrated from hexanes to form a solid which was recrystalized from EtOAc/Hexanes to give pure 14 (0.174 g, 75%): MS: APCI (AP+): 504 (M+H)⁺; CHN calc'd for C₂₄H₂₃Cl₁F₁N₃O₄S₁+0.05H₂O: calc'd.: % C 57.09; % H 4.61; % N 8.32. Found: % C 57.18; % H 4.55; % N 8.17.

### Example 15

### 2-[3-(4-Chloro-phenyl)-ureido]-3-hydroxy-2-hydroxymethyl-N-(2′-sulfamoyl-biphenyl-4-yl)-propionamide (compound 15)

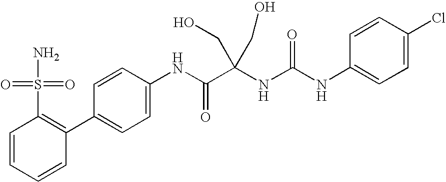

Step 1: [5-(4-Bromo-phenylcarbamoyl)-2-phenyl-[1,3]dioxan-5-yl]-carbamic acid tert-butyl ester (15a). 5-tert-Butoxycarbonylamino-2-phenyl-[1,3]dioxane-5-carboxylic acid (0.985 g, 3.05 mmol), 4-bromoaniline (0.517 g, 3.05 mmol) and EEDQ (0.902 g, 3.65 mmol) were dissolved in dry CHCl₃ (15 mL). Triethylamine (0.64 mL, 4.56 mmol) was added, and the solution heated at reflux for 22 h. The reaction was allowed to cool to RT and then concentrated to a white solid which was partitioned between EtOAc and water. The aqueous layer was then extracted with EtOAc again, and the organic layers combined, washed with brine and dried over MgSO₄. Concentration of the organic layer and purification of the crude product by recrystallization (EtOAc/Hex) revealed 15a (0.53 g, 36%) as a white solid:

Step 2: [5-(2′-tert-Butylsulfamoyl-biphenyl-4-ylcarbamoyl)-2-phenyl-[1,3]dioxan-5-yl]-carbamic acid tert-butyl ester (15b). A mixture of 15a (0.525 g, 1.09 mmol), 2-tert-butylsulfamoylphenyl boronic acid (0.339 g, 1.31 mmol), tetrabutylammonium bromide (0.018 mg, 0.055 mmol), sodium carbonate (0.232 g, 2.19 mmol), and water (1 mL) in toluene (11 mL) was degassed with a stream of argon. Tetrakis(triphenylphosphine) Pd(0) (0.127 g, 0.109 mmol) was then added, and the mixture heated at reflux under an argon atmosphere for 4.5 h. The resulting solution was allowed to cool to RT and concentrated to a solid which was partitioned between EtOAc and water. The organic layer was separated and washed with brine and dried over MgSO₄. Concentration of the organic layer, and purification of the resulting residue by MPLC resulted in the product 15b (0.345 g, 52%) as a white solid.

Step 3: [1-(2′-tert-Butylsulfamoyl-biphenyl-4-ylcarbamoyl)-2-hydroxy-1-hydroxy-methylethyl]-carbamic acid-tert-butyl ester (15c). A solution of 15b (0.255 g, 0.418 mmol) was dissolved in EtOH (10 mL) and stirred under a hydrogen atmosphere (5 atm, RT) over 20% palladium/carbon for 19 h. The reaction was filtered, and the catalyst washed with THF. The combined filtrates were then concentrated under reduced pressure resulting in 15c (0.217 g) as an impure, white solid which was used directly in subsequent reactions.

Step 4: N-(2′-tert-Butylsulfamoyl-biphenyl-4-yl)-2-[3-(4-chloro-phenyl)-ureido]-3-hydroxy-2-hydroxymethyl-propionamide (15d). Into a solution of 15c (0.217 g, 0.418 mmol) in dry DCM (4 mL) was added TFA (1 mL), and the solution stirred at RT for 1 h. The solution was then concentrated under reduced pressure. Hexanes were added, and the residue concentrated again under reduced pressure. The resulting oil was then dried under vacuum. The crude product was dissolved in dry THF (4 mL) and cooled to 0° C. in an ice bath. Triethylamine (0.233 mL, 1.67 mmol) was added followed by the addition of 4-chlorophenyl isocyanate (0.064 g, 0.418 mmol). The reaction was allowed to stir at RT for 1 h before being concentrated under reduced pressure, and the resulting crude solid purified by MPLC to yield the product as an oil. The product was recrystalized from EtOAc/hexanes to form 15d (0.135 g, 56%) as a white solid.

Step 5: 2-[3-(4-Chloro-phenyl)-ureido]-3-hydroxy-2-hydroxymethyl-N-(2′-sulfamoyl-biphenyl-4-yl)-propionamide (15). A solution of 15d (0.135 g, 0.234 mmol) and TFA (4 mL) was stirred at RT for 2 h. The reaction was then concentrated under reduced pressure, and the resulting oil purified by MPLC to yield the product 15 (0.081 g, 67%) as a white solid which was further purified by recrystalization from EtOAc/hexanes: APCI HRMS: calc'd for C₂₃H₂₄Cl₁N₄O₆S (M+H)⁺: 519.1105. Found: 519.1104.

### Example 16

### 4-[3-(4-Chloro-phenyl)-ureido]-tetrahydro-pyran-4-carboxylic acid (3-fluoro-2′-sulfamoyl-biphenyl-4-yl)-amide (compound 16)

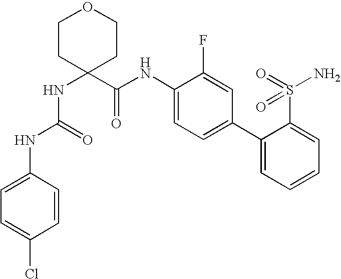

Step 1:[4-(2′-tert-Butylsulfamoyl-3-fluoro-biphenyl-4-ylcarbamoyl)-tetrahydro-pyran-4-yl]-carbamic acid tert-butyl ester (16a). 4-tert-Butoxycarbonylamino-tetrahydro-pyran-4-carboxylic acid (1.00 g, 4.08 mmol), 4′-amino-3′-fluoro-biphenyl-2-sulfonic acid tert-butylamide (1.31 g, 4.08 mmol), and EEDQ (1.21 g, 4.89 mmol) were dissolved in dry CHCl₃ (40 mL). Triethylamine (0.852 mL, 6.12 mmol) was added and the solution heated at reflux for 26 h. The solution was cooled, EtOAc added, and then washed sequentially with 10% aq. citric acid, 1N NaOH, water, and brine before drying over MgSO₄. Concentration of the solution under reduced pressure and purification of the crude product by flash chromatography revealed 16a (0.972 g, 43%) as a white foam.

Step 2: 4-[3-(4-Chloro-phenyl)-ureido]-tetrahydro-pyran-4-carboxylic acid (3-fluoro-2′-sulfamoyl-biphenyl-4-yl)-amide (16). To 16a (0.300 g, 0.546 mmol) was added TFA (8 mL), and the solution stirred at reflux for 0.75 h. The solution was then concentrated under reduced pressure and dried under vacuum to yield a crude oil which was dissolved in dry THF (6 mL) and cooled to 0° C. in an ice bath. Triethylamine (0.380 mL, 2.73 mmol) was then added followed by 4-chlorophenyl isocyanate (0.084 g, 0.546 mmol). The reaction was allowed to stir at RT for 1 h before concentrating under reduced pressure. The resulting crude product was purified by flash chromatography and lyophilized from MeCN/H₂O to yield 16 (0.205 g, 69%) as a white solid: MS: APCI (AP+): 547 (M)+; CHN calc'd for C₂₅H₂₄Cl₁F₁N₄O₅S₁: % C 53.94; % H 4.38; % N 9.99. Found: % C 54.01; % H 4.41; % N 9.78.

### Example 17

### 4-[3-(4-Chloro-phenyl)-ureido]-tetrahydro-thiopyran-4-carboxylic acid (3-fluoro-2′-sulfamoyl-biphenyl-4-yl)-amide (Compound 17)

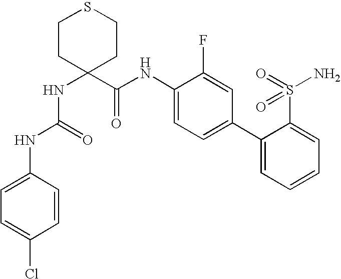

Step 1: [4-(4-Bromo-2-fluoro-phenylcarbamoyl)-tetrahydro-thiopyran-4-yl]-carbamic acid tert-butyl ester (17a). 4-tert-Butoxycarbonylamino-tetrahydro-thiopyran-4-carboxylic acid (0.490 g, 1.88 mmol), 4-bromo-2-fluoroaniline (0.356 g, 1.88 mmol), and EEDQ (0.556 g, 2.25 mmol) were dissolved in dry CHCl₃ (19 mL). Triethylamine (0.392 mL, 2.81 mmol) was added and the solution heated at reflux for 48 h before cooling and adding EtOAc. This was washed sequentially with 10% aq. citric acid, 1N NaOH, water, and then brine before drying the solution over MgSO₄. Concentration of the solution under reduced pressure and purification of the crude product by flash chromatography revealed 17a (0.172 g, 21%) as a white solid.

Step 2: [4-(2′-tert-Butylsulfamoyl-3-fluoro-biphenyl-4-ylcarbamoyl)-tetrahydro-thiopyran-4-yl]-carbamic acid tert-butyl ester (17b). Bromide 17a (0.172 g, 0.397 mmol) was combined with 2-tert-butylphenylsulfamoyl boronic acid (0.153 g, 0.595 mmol), K₃PO₄(0.126 g, 0.595 mmol), and anhydrous DMF (5 mL). The mixture was degassed with argon before and after the addition of tetrakis(triphenylphosphine)palladium(0) (0.046 g, 0.040 mmol). The mixture was stirred at 110° C. for 17 h before cooling and partitioning between EtOAc and H₂O. The organic layer was washed with brine and then dried over MgSO₄. Concentration of the solution under reduced pressure and purification of the crude product by flash chromatography revealed 17b (0.050 g, 22%) as a white foam.

Step 3: 4-[3-(4-Chloro-phenyl)-ureido]-tetrahydro-thiopyran-4-carboxylic acid (3-fluoro-2′-sulfamoyl-biphenyl-4-yl)-amide (17). To 17b (0.050 g, 0.088 mmol) was added TFA (5 mL) and the solution stirred at reflux for 2 h. The solution was then concentrated under reduced pressure and dried under vacuum to yield a crude oil which was dissolved in dry THF (2 mL) and cooled to 0° C. in an ice bath. Triethylamine (0.061 mL, 0.440 mmol) was then added followed by 4-chlorophenyl isocyanate (0.014 g, 0.088 mmol). The reaction was allowed to stir at RT for 2.5 h before concentrating under reduced pressure. The crude product was then purified by flash chromatography. The resulting solid was azeotroped with CHCl₃ and lyophilized from MeCN/H₂O to reveal 17 (0.030 g, 61%) as a white solid: APCI HRMS: calc'd for C₂₅H₂₄Cl₁F₁N₄O₄S₂ (M+H)+: 563.0990.

Found: 563.0994.

### Example 18

### (1S,2S)-1-[3-(4-Chloro-phenyl)-ureido]-2-hydroxymethyl-cyclopropanecarboxylic acid (3-fluoro-2′-methanesulfonyl-biphenyl-4-yl)-amide (Compound 18).

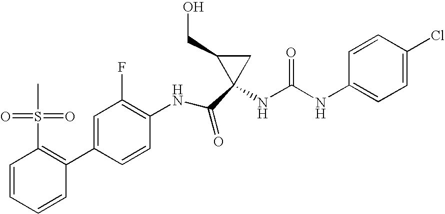

### Step 1: (1S,2S)-1-[(tert-butoxy)carbonylamino]-2-(hydroxymethyl)cyclopropane-carboxylic acid (18a)

Into the vigorously stirred solution of (2-Oxo-3-oxa-bicyclo[3.1.0]hex-1-yl)-carbamic acid tert-butyl ester (prepared according to K. Burgess et al. J. Org. Chem., 1992, 57, 5931; and D. R. Morton et al.. J. Org. Chem., 1978, 57, 2101) (2.98 g, 14.0 mmol) in THF (75 mL), a solution of lithium hydroxide monohydrate (0.705 g, 16.8 mmol) in water (75 mL) was added and the reaction stirred for 4 h. The THF was removed under reduced pressure, diluted with water (75 mL) and pH adjusted to 3 with 1M citric acid. The resulting solution was extracted with EtOAc (100 mL). To the aqueous layer, brine (200 mL) was added and further extracted with ethyl acetate (2×100 mL). The combined organic extracts were washed with brine, dried over Na₂SO₄ and concentrated under reduced pressure. The resulting white solid 18a (2.52 g) was used for the subsequent step without further purification.

### Step 2: (1,S,2S)-1-[(tert-Butoxy)carbonylamino]-2-[(1,1,2,2-tetramethyl-1-silapropoxy)methyl]cyclopropanecarboxylic acid (18b)

Into a solution of 18a (0.9 g, 3.9 mmol) in dry DMF (13 mL) was added tert-butyldimethylsilyl chloride (1.29 g, 8.58 mmol) and imidazole (1.09 g, 16.0 mmol), and the solution was stirred for 17 h. The reaction mixture was poured into brine (165 mL) and extracted with ether (2×100 mL). The organic extract was cooled to 0° C. and washed with 0.5 N HCl (75 mL), brine and dried over Na₂SO₄. Concentration of the solution under reduced pressure provided bis-silylation product.

Into the crude product was added methyl alcohol (50 mL), THF (16.5 mL), a solution of potassium carbonate (1.65 g, 11.9 mmol) in water (16.5 mL), and the reaction stirred for 1 h. The volume of the reaction mixture was reduced to one-fourth and then diluted with brine (50 mL). The cloudy mixture was cooled to 0° C. in an ice bath and acidified with 1 M potassium bisulfate solution to pH 5.0 and subsequently extracted with ether (2×100 mL). The combined organic extracts were washed with brine before drying over Na₂SO₄. Concentration under reduced pressure gave 18b (1.35 g), as a pale yellow solid that was used for the next step without further purification.

### Step 3: {(1S,2S)-1-[(tert-Butoxy)carbonylamino]-2-[(1,1,2,2-tetramethyl-1-silapropoxy)methyl]cyclopropyl}-N-{2-fluoro-4-[2-(methylsulfonyl)phenyl]phenyl}carboxamide (18c)

Into a solution of 18b (1.34 g, 3.88 mmol) in dry chloroform (30 mL), 1-(4-amino-3-fluorophenyl)-2-(methylsulfonyl)benzene (1.03 g, 3.88 mmol) and EEDQ (1.22 g, 4.93 mmol) were added, followed by triethylamine (0.87 mL, 6.21 mmol). The solution was heated to reflux for 24 h. It was allowed to cool and further diluted with chloroform, cooled to 0° C. and washed sequentially with 5% HCl, brine and dried over Na₂SO₄. Concentration of the solution under reduced pressure and purification of the crude product by flash chromatography over silica gel using 30% acetone/hexane gave 18c (1.03 g) as white solid.

### Step 4: (1S,2S)-1-{[(4-Chlorophenyl)amino]carbonylamino}-2-(hydroxymethyl cyclopropyl)-N-{2-fluoro-4-[2-(methylsulfonyl)phenyl]phenyl}carboxamide (18)

Into a stirred solution 18c (0.977 g, 1.65 mmol) in dry dichloromethane (20 mL), iodotrimethylsilane (0.563 mL, 3.96 mmol) was added dropwise. After 20 min, the reaction mixture was quenched with methyl alcohol (0.641 mL) and stirred for 5 min. The reaction mixture was then concentrated under reduced pressure and dried under vacuum to afford the corresponding fully deprotected amino alcohol as pale yellow solid which was used in the next step.

The crude product was dissolved in dry THF (25 mL) and cooled to 0° C. in an ice bath. Triethylamine (0.460 mL, 3.3 mmol) was added followed by 4-chlorophenyl isocyanate (0.253 g, 1.65 mmol). The solution was stirred at 0° C. for 30 min and at room temperature for 3 h. After concentration under reduced pressure, the crude product was taken up in chloroform (100 mL), cooled to 0° C. and washed sequentially with 5% HCl, brine, dried over Na₂SO₄ and concentrated under reduced pressure. The resulting crude product was purified by flash chromatography using silica gel and a gradient of acetone/hexane (30%-50%) to afford compound 18 (0.475 g) as a white solid. MS (ES⁻): m/e 529.7; CHN calculated for C₂₅H₂₃ClFN₃O₅S: C, 56.49%; H, 4.36%; N, 7.90%. Found: C, 56.55%; H, 4.36%; N, 7.71%.

### Example 19

### (1S,2S)-1-[3-(4-Chloro-phenyl)-ureido]-2-hydroxymethyl-cyclopropanecarboxylic acid [2-fluoro-4-(2-oxo-piperidin-1-yl)-phenyl]-amide (Compound 19)

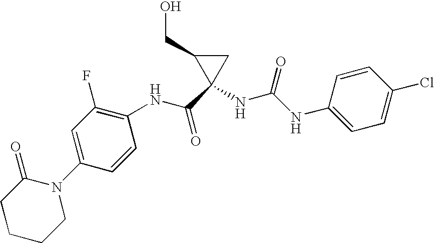

Compound 19 was synthesized following the general procedure as described for Example 18 with the only difference being the use of 1-(4-Amino-3-fluoro-phenyl)-piperidin-2-one in step 3. MS (ES+): m/e 474.8.

### Example 20

### (1R,2S)-1-[3-(4-Chloro-phenyl)-ureido]-2-hydroxymethyl-cyclopropanecarboxylic acid (3-fluoro-2′-methanesulfonyl-biphenyl-4-yl)-amide (Compound 20)

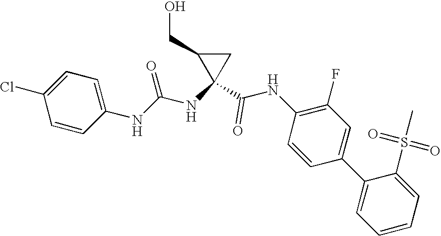

### Step 1: (1R,2S)-2-(acetyloxymethyl)-1-[(tert-butoxy)carbonylamino]cyclopropane-carboxylic acid (20a)

Into a solution of (Z)-1-{[(tert-butoxy)carbonyl]amino}-2-(hydroxymethyl) cyclopropanecarboxylic acid (prepared according to Michael C. Pirrung, Stevens E. Dunlap, Uwe P. Trinks. Helv. Chimica. Acta., 1989, 72, 1301-1310 and R. S. Lott. J.C.S. Chem. Comm., 1979, 495.) (1.3 g, 4.32 mmol) in dry pyridine (6.38 mL) was added acetic anhydride (0.65 g, 6.4 mmol) and the solution wa stirred at room temperature for 22 h. To this solution EtOAc (150 mL), 2N HCl (75 mL) and brine (75 mL) were added, and the two layers were separated. The organic layer was dried over Na₂SO₄ and then reduced under reduced pressure to give compound 20a (2.00 g) as a brown oil.

### Step 2: (1R,2S)-2-[(tert-butoxy)carbonylamino]-2-(N-{2-fluoro-4-[2-(methylsulfonyl)-phenyl]phenyl}carbamoyl)cyclopropyl]methyl acetate (20b)

To dry chloroform (15 mL), 20a (2.0 g, 7.34 mmol), 1-(4-amino-3-fluorophenyl)-2-(methylsulfonyl)benzene (2.2 g, 8.34 mmol) and EEDQ (2.3 g, 9.34 mmol) were added, and the solution was heated at reflux for 16 h. The reaction was cooled and then diluted with EtOAc. This mixture was washed with 2N HCl (3×15 mL), 1N NaOH (3×15 mL), water (3×10 mL), brine (3×15 mL) and dried over Na₂SO₄. Concentration of the solution under reduced pressure and purification of the crude product by flash chromatography on silica gel using hexanes and EtOAc mixture (7:3 v/v) as eluent gave pure 20b (1.1 g) as a white foamy solid.

### Step 3: (1R,2S)-2-{[(4-chlorophenyl)amino]carbonylamino}-2-(N-{2-fluoro-4-[2-(methylsulfonyl)phenyl]phenyl}carbamoyl)cyclopropyl}methyl acetate (20c)

To a solution of 20b (1.1 g, 1.83 mmol) in dry dichloromethane (7 mL) was added iodotrimethylsilane (0.5 mL, 3.96 mmol). The solution was stirred for 10 min and then quenched with methanol. The mixture was concentrated under reduced pressure and dried under vacuum. The crude product was used directly in the next reaction.

The crude 2-amino-2-(N-{2-fluoro-4-[2-(methylsulfonyl) phenyl]phenyl}carbamoyl)cyclopropyl]methyl acetate (0.800 g, 1.82 mmol) was dissolved in THF (10 mL) and cooled to 0° C. in an ice bath, and 4-chlorophenyl isocyanate (0.306 g, 2.0 mmol) was added followed by triethylamine (0.695 mL, 5 mmol). The reaction mixture was stirred overnight and concentrated under reduced pressure. The residue was dissolved in EtOAc, washed with NaHCO₃, (3×15 mL), water (3×10 mL), brine (3×15 mL) and dried over Na₂SO₄. The crude product was purified by flash chromatography using hexane: EtOAc (1:1 v/v) to afford white solid 20c (0.500 g).

### Step 4: (1R,2S)-1-{[(4-chlorophenyl)amino]carbonylamino}-2-(hydroxymethyl)cyclopropyl)-N-{2-fluoro-4-[2-(methylsulfonyl)phenyl]phenyl}carboxamide (20d)

The compound 20c (0.500 g, 0.84 mol) was dissolved in THF (5 mL) and potassium trimethylsilanoate (0.433 g, 3.38 mmol) was added. The mixture was stirred at room temperature for 4 h and was concentrated under reduced pressure. The crude product was purified by preparative thin layer chromatography eluting with a mixture of hexane: acetone (1:1 v/v). The compound 20d was obtained as a white solid (0.298 g). MS (ES⁺): m/e 531 (M⁺); CHN calculated for C₂₅H₂₃N₃O₅ClFS: C, 56.49 %; H, 4.36% ; N, 7.70%.

Found: C, 57.36 %; H. 4.93 %; N, 6.75 %.

### Example 21

### (1R,2S)-1-[3-(4-Chloro-phenyl)-ureido]-2-hydroxymethyl-cyclopropanecarboxylic acid [2-fluoro-4-(2-oxo-piperidin-1-yl)-phenyl]-amide (Compound 21).

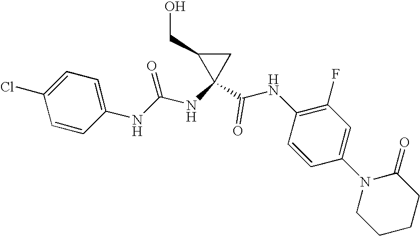

Compound 21 was synthesized following the general procedure as described for Example 20 with the only difference being the use of 1-(4-Amino-3-fluoro-phenyl)-piperidin-2-one in step 3. MS (ES−): m/e 472.8

### Example 22

### 3-[3-(4-Chloro-phenyl)-ureido]-3-(3-fluoro-2′-methanesulfonyl-biphenyl-4-ylcarbamoyl)-pyrrolidine-1-carboxylic acid benzyl ester (Compound 22)

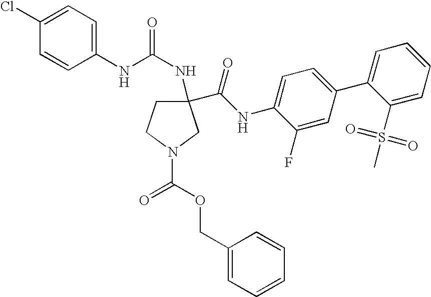

### Step 1: Methyl 3-amino-1-benzylpyrrolidine-3-carboxylate (22a)

3-(Benzhydrylidene-amino)-1-benzyl-pyrrolidine-3-carboxylic acid methyl ester (prepared according to C. Balsamini et al. Synthesis 1990, 779-781; and O. Mamoun et al. Synth. Comm., 1995, 25, 1295) (28.0 g, 70.3 mmol) was dissolved in diethyl ether (280 mL) and cooled in an ice-salt bath. Cold 1 M hydrochloric acid (190 mL, 190 mmol) was added over 40 min. The reaction mixture was stirred vigorously in the ice-salt bath for 30 min and then at RT for 20 h. The ether layer was decanted from the aqueous layer. Fresh diethyl ether (3×70 mL) was added, stirred and decanted. The aqueous layer was basified (pH 8) with saturated sodium bicarbonate and extracted with dichloromethane (4×100 mL). After drying over Na₂SO₄, the solvent was removed under reduced pressure. Compound 22a (15.1 g) was obtained as a thick oil.

### Step 2: Methyl 3-[(tert-butoxy)carbonylamino)-1-benzylpyrrolidine-3-carboxylate ( 22b )

Compound 22a (0.40 g, 1.72 mmol) was dissolved in dry dichloromethane (10 mL) and cooled in an ice-salt bath. Di-tert-butyldicarbonate (0.42 g, 1.90 mmol) dissolved in dry dichloromethane (10 mL) was added dropwise. This mixture was first stirred in the ice-salt bath for 5 min and then at RT for 36 h. Removal of solvents provided a thick oil which was purified by silica gel column chromatography with hexanes-THF gradient. Compound 22b (0.31 g) was isolated as a thick oil.

### Step 3: Methyl 3-[(tert-butoxy)carbonylamino]-1-[benzyloxy carbonyl]pyrrolidine-3-carboxylate ( 22c )

Compound 22b (0.30 g, 0.898 mmol) was dissolved in dry dichloromethane (20 mL) and cooled in an ice-salt bath. A solution of benzyl chloroformate (0.306 g, 1.796 mmol) in dichloromethane (10 mL) was added dropwise. The solution was stirred in the ice-salt bath for another 15 min, then at RT for 15 h and finally at 40-45° C. for 3 h. Concentration under reduced pressure gave a thick oil. Purification by silica gel column chromatography with hexanes-THF gradient provided compound 22c (0.31 g) as a thick oil.

### Step 4: 3-[(Tert-butoxy)carbonylamino]-1-[benzyloxycarbonyl]pyrrolidine-3-carboxylic acid (22d)

Compound 22c (0.30 g, 0.793 mmol) was dissolved in THF-water (20 mL/10 mL). Lithium hydroxide monohydrate (0.066 g, 1.587 mmol) was added, and the reaction stirred at RT for 16 h. The volume of the reaction mixture was reduced to one half under reduced pressure. After diluting with water (10 mL) and extracting with hexanes, the aqueous layer was acidified (pH 5) with 1 M citric acid and the volume reduced again to one half under reduced pressure. After standing overnight at room temperature, the resulting precipitate was collected and dried under high vacuum to give compound 22d (0.275 g) as a white solid.

### Step 5: Phenylmethyl-3-[(tert-butoxy)carbonylamino]-3-(N-{2-fluoro-4-[2-methylsulfonyl)phenyl]phenyl}carbamoyl)pyrrolidinecarboxylate (22e)

Triethylamine (1.05 g, 10.3 mmol) in dry chloroform (100 mL) was added to a mixture of compound 22d (1.985 g, 5.45 mmol), 1-(4-amino-3-fluorophenyl)-2-methylsulfonylbenzene (1.58 g, 6.01 mmol) and EEDQ (1.687 g, 6.83 mmol). The resulting solution was heated at reflux for 20 h. Solvent was removed under reduced pressure. The remaining residue was dissolved in dichloromethane (100 mL) and washed with 5 N HCl (4×50 mL). The organic layers were successively washed with water, saturated sodium bicarbonate, water, brine. After drying over anhydrous Na₂SO₄, dichloromethane was removed under reduced pressure to provide a solid. Compound 22e (0.761 g) was isolated as a white solid after silica gel chromatography using hexanes-THF-methanol gradient.

### Step 6: Phenylmethyl 3-amino-3-(N-{2-fluoro-4-[2-(methyl sulfonyl)phenyl)phenyl]-phenyl}carbamoyl)pyrrolidinecarboxylate (22f)

A cold solution of trifluoroacetic acid (5 mL) in dry chloroform (2 mL) was added dropwise to a solution of compound 22e (0.19 g, 0.31 mmol) in dry chloroform ( 2 mL) cooled in an ice bath. The solution was stirred in the ice bath for another 15 min and then at RT for 16 h. Solvents and excess trifluoroacetic acid were removed under vacuum. After dissolving the solid in dichloromethane (20 mL), it was washed successively with saturated sodium bicarbonate (2×10 mL), water, brine and dried over anhydrous Na₂SO₄. Removal of dichloromethane under reduced pressure and then under high vacuum provided compound 22f (0.145 g) as a white solid.

### Step 7: 3-[3-(4-Chloro-phenyl)-ureido]-3-(3-fluoro-2′-methanesulfonyl-biphenyl-4-ylcarbamoyl)-pyrrolidine-1-carboxylic acid benzyl ester (22)

To a mixture of compound 22f (0.230 g, 0.450 mmol) and p-chlorobenzyl isocyanate (0.076 g, 0.496 mmol) was added dry THF (15 mL) followed by triethylamine (0.145 mL). The resulting solution was stirred under nitrogen at RT for 14 h. After removal of solvents under reduced pressure, the remaining solid was purified by silica gel chromatography using hexanes-dichloromethane gradient. Compound 22 (0.156 g) was isolated as a white solid.

MS (ES+): m/e 665.31 (M+1).

### Example 23

### 2-[3-(4-Chloro-phenyl)-1-cyclopropylmethyl-ureido]-N-(3-fluoro-2′-methanesulfonyl-biphenyl-4-yl)-acetamide (Compound 23).

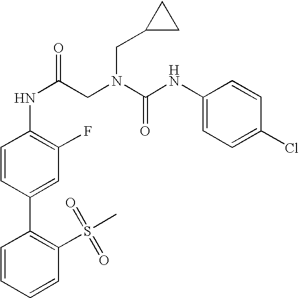

Step 1: 2-Bromo-N-(3-fluoro-2′-methanesulfonyl-biphenyl-4-yl)-acetamide (23a). To a solution of 3-fluoro-2′-methanesulfonyl-biphenyl-4-ylamine (2 g, 7.55 mmol) in anhydrous dichloromethane (0.1 M, 75 mL), cooled to 0° C. (ice bath), was added triethylamine (1.25 mL, 9.06 mmol), followed by the dropwise addition of bromoacetyl chloride (1.43g, 9.06 mmol). The reaction mixture was allowed to warm to room temperature and stirred for 2 h. The reaction was diluted with EtOAc (100 mL). The organic solution was extracted with 1N HCl (30 mL), washed with brine (30 mL), dried over MgSO₄, and concentrated to give a pale-white solid 23a (2.68 g, 92%). MS(APCI+): m/z⁺387 (M+1).

Step 2: 2-(Cyclopropylmethyl-amino)-N-(3-fluoro-2′-methanesulfonyl-biphenyl-4-yl)-acetamide (23b). A mixture of 23a (0.193 g, 0.5 mmol), diisopropylethyl amine (0.11 mL, 0.75 mmol), and (aminomethyl)cyclopropane (0.072 g, 1 mmol) in dichloromethane (5 mL) was stirred for 15 h at room temperature. The reaction was quenched with the addition of benzaldehyde polystyrene resin (1 g, L: 1.2 mmol/g), and the suspension was stirred for 3 hours. The resin was filtered and washed with dichloromethane (15 mL), followed by methanol (15 mL), twice. The filtrates were combined and concentrated. The residue was dissolved in ethyl acetate (20 mL), extracted with 1N HCl (15 mL), and washed with saturated sodium bicarbonate (20 mL). The organic solution was dried over MgSO₄, and concentrated to give a yellow oil. The product was purified by reverse phase chromatography (Varian Megabond Elut C 18 column; eluent: 20% acetonitrile −80% water in the presence of 0.1% TFA). The residual water in the product was azeotroped with toluene. The dried product was dissolved in dichloromethane (10 mL), washed with saturated sodium bicarbonate (10 mL), dried over MgSO₄ and concentrated to give 23b (0.180 g, 95%) as a white solid.

Step 3: 2-[3-(4-Chloro-phenyl)-1-cyclopropylmethyl-ureido]-N-(3-fluoro-2′-methanesulfonyl-biphenyl-4-yl)-acetamide (23). A mixture of 23b (0.180 g, 0.47 mmol), triethylamine (1 mmol), and 4-chlorophenyl isocyanate (0.87 g, 0.57 mmol ) in anhydrous dichloromethane (5 mL) was allowed to stir for 1.5 h at room temperature. The reaction was quenched with the addition of tris-amine resin (0.5 g, L: 1.6 mmol/g) and the suspension was allowed to stir for an additional 30 min. The resin was filtered off and washed with dichloromethane (20 mL), followed by methanol (20 mL), twice. The filtrates were combined and concentrated. The residue was dissolved in dichloromethane (30 mL), extracted with 1N HCl (15 mL). The organic layer was dried over MgSO₄ and concentrated. Purification by flash column chromatography (silica gel; eluent: 50% ethyl acetate in hexanes) gave compound 23 (0.048 g, 19%) as a white foam, LCMS: m/z⁺530 (M+1).

### Example 24

### 2-[3-(4-Chloro-phenyl)-1-(2-methoxy-ethyl)-ureido]-N-(3-fluoro-2′-methanesulfonyl-biphenyl-4-yl)-acetamide (Compound 24)

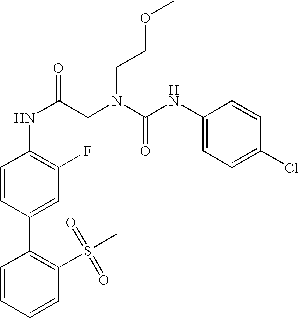

2-[3-(4-Chloro-phenyl)-1-(2-methoxy-ethyl)-ureido]-N-(3-fluoro-2′-methanesulfonyl-biphenyl-4-yl)-acetamide 24 was synthesized according to Example 23, Step 2, by substituting 2-methoxyethylamine for (aminomethyl)cyclopropane. The title compound 24 (0.089 g, 35%) was obtained as a white solid; LCMS: m/z 535⁺ (M+1).

### Example 25

### 2-[3-(4-Chloro-phenyl)-1-isobutyl-ureido]-N-(3-fluoro-2′-methanesulfonyl-biphenyl-4-yl)-acetamide (Compound 25)

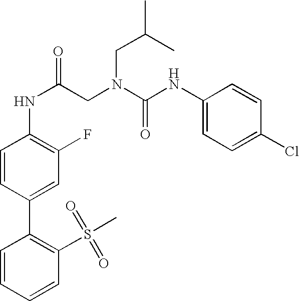

2-[3-(4-Chloro-phenyl)-1-isobutyl-ureido]-N-(3-fluoro-2′-methanesulfonyl-biphenyl-4-yl)-acetamide 25 was synthesized according to Example 23, Step 2, by substituting isobutyl amine for (aminomethyl)cyclopropane. The title compound 25 (0.084 g, 34%) was obtained as a white solid; LCMS: m/z 535⁺ (M+1).

### Example 26

### 2-[3-(4-Chloro-phenyl)-1-(2-dimethylamino-ethyl)-ureido]-N-(3-fluoro-2′-methanesulfonyl-biphenyl-4-yl)-acetamide (Compound 26)

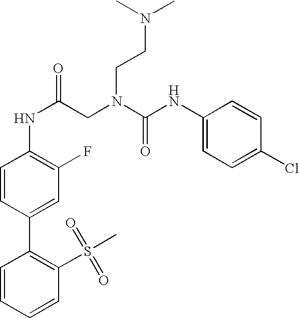

2-[3-(4-Chloro-phenyl)-1-(2-dimethylamino-ethyl)-ureido]-N-(3-fluoro-2′-methanesulfonyl-biphenyl-4-yl)-acetamide 26 was synthesized according to Example 23, Step 2, by substituting N,N-dimethylethylenediamine for (aminomethyl)cyclopropane. The title compound 26 (0.048 g, 19%) was obtained as a white solid; LCMS: m/z⁺ 547 (M+1).

### Example 27

### 2-[1-Benzyl-3-(4-chloro-phenyl)-ureido]-N-(3-fluoro-2′-methanesulfonyl-biphenyl-4-yl)-acetamide (Compound 27)

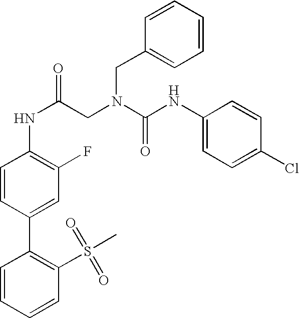

2-[1-Benzyl-3-(4-chloro-phenyl)-ureido]-N-(3-fluoro-2′-methanesulfonyl-biphenyl-4-yl)-acetamide 27 was synthesized according to Example 23, Step 2, by substituting benzylamine for (aminomethyl)cyclopropane. The title compound 27 (0.068 g, 26%) was obtained as a white solid; LCMS: m/z⁺ 567 (M+1).

### Example 28

### 2-[3-(4-Chloro-phenyl)-1-(4-methoxy-benzyl)ureido]-N-(3-fluoro-2′-methanesulfonyl-biphenyl-4-yl)-acetamide (Compound 28)

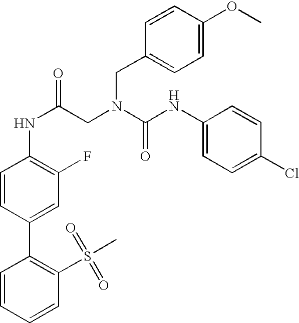

2-[3-(4-Chloro-phenyl)-1-(4-methoxy-benzyl)ureido]-N-(3-fluoro-2′methanesulfonyl-biphenyl-4-yl)-acetamide 28 was synthesized according to Example 23, Step 2, by substituting 4-methoxybenzylamine for (aminomethyl)cyclopropane. The title compound 28 (0.058 g, 35%) was obtained as a yellow solid; LCMS: m/z⁺ 597 (M+1).

### Example 29

### 2-[3-(4-Chloro-phenyl)-1-(2-methoxy-ethyl)-ureido]-N-[2-fluoro-4-(2-oxo-piperidin-1-yl)-phenyl]-acetamide (Compound 29)

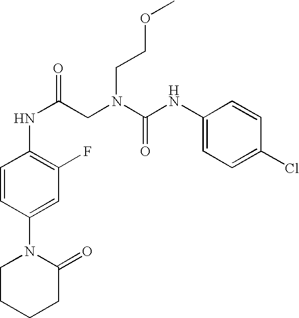

Compound 29 was synthesized according to Example 23, Step 1, by substituting 1-(4-amino-3-fluoro-phenyl)-piperidin-2-one for 3-fluoro-2′-methanesulfonyl-biphenyl-4-ylamine, and Step 2, by substituting 2-methoxyethylamine for (aminomethyl) cyclopropane. The title compound 29 (0.084 g, 37%) was obtained as a white solid; LCMS: m/z⁺ 477 (M+1).

It will be appreciated by those skilled in the art that compounds of the invention having one or more chiral centers may exist in and be isolated in optically active and racemic forms. Some compounds may exhibit polymorphism. It is to be understood that the present invention encompasses any racemic, optically-active, polymorphic, geometric, tautomeric, or stereoisomeric form, or mixtures thereof, of a compound of the invention, which possess the useful properties described herein; it being well known in the art how to prepare optically active forms (for example, by resolution of the racemic form by recrystallization techniques, by synthesis from optically-active starting materials, by chiral synthesis, or by chromatographic separation using a chiral stationary phase) and how to determine activity using the standard tests described herein, or using other similar tests which are well known in the art.

In addition, certain of the compounds of the present invention can exist in unsolvated forms as well as solvated forms, including hydrated forms. In general, the solvated forms, including hydrated forms, are equivalent to unsolvated forms and are intended to be encompassed within the scope of the present invention.

Biological Assays

The invention compounds have demonstrated factor Xa inhibitory activity in the standard assays commonly employed by those skilled in the art.

a. Determination of Factor Xa IC₅₀

The ability of compounds to act as inhibitors of human factor Xa catalytic activity was assessed by determination of that concentration of test substance that inhibited by 50% (IC₅₀) the ability of human factor Xa to cleave the fluorogenic substrate F-S2765 (N-α-Z-D-Arg-Gly-Arg-pNA HCl, California Peptide Research). Inhibitory amounts of compounds of the invention include those with IC₅₀ values of 1 to 500 nM for example. The IC₅₀ was determined at 3 pM and/or 30 pM concentrations of human factor Xa (Enzyme Research Laboratories). These concentrations were achieved by diluting a stock solution of human factor Xa in the appropriate amount of buffer containing 10 mM HEPES, 150 mM NaCl, and 0.1% BSA at pH 7.4 (HBSA buffer). Accordingly, 73 μL of the factor Xa/buffer solution was added to 2.5 μL of DMSO-reconstituted compound and incubated for 55 minutes at room temperature. After warming to 37° C. for am additional 5 minutes, 50 μL of rewarmed substrate was added and the IC₅₀ determined by monitoring the increase in absorbance at 390 nm excitation/460 nm emission (455 nm cutoff) in a fluorometric plate reader or 30 minutes. Results of the IC₅₀ at 3 pM and 30 pM enzyme concentrations are provided in Table 1.

b. Determination of Prothrombin Time (PT)

The prothrombin time (PT) is a measure of extrinsic and common pathway factors. In this assays, human tissue thromboplastin is added to human plasma which activates pathway factors, including factor X activation to factor Xa, leading to clot formation. Significant inhibition of factor Xa, by a small molecule inhibitor, will reduce the conversion of prothrombin to thrombin and thereby increase the time to clot formation in this assay. The value 2 X PT is the concentration of the inhibitor required to increase the clotting time by 2-fold. These values are also presented in Table 1.

Given the data presented in Table 1, compounds of the present invention act as inhibitors of Factor Xa. Accordingly, the compounds of the present invention are useful in pharmaceutical formulations for preventing and treating thrombotic disorders. Such disorders include venous thrombosis, deep vein thrombosis, thrombophlebitis, arterialembolism, coronary and cerebral arterial thrombosis, cerebral embolism, kidney embolism, pulmonary embolism, first or recurrent myocardial infarction, unstable angina, cerebral infarction, stroke, and atherosclerosis.

TABLE 1

| | FXa 3 pM | FxXa 30 pM | PT Conc. 2X |
|---|---|---|---|
| EXAMPLE | IC₅₀ (nM) | IC₅₀ (nM) | Prolong. (μM) |
| 1 | 38 | | 13.45 |
| 2 | 33 | | |
| 3 | 22 | | |
| 4 | | 255 | |
| 5 | | 36 | |
| 6 | | 192 | |
| 7 | 20 | | |
| 8 | | 287 | |
| 9 | | 44 | |
| 10 | 16 | | |
| 11 | | 66 | |
| 12 | | 20 | |
| 13 | | 17% @ 1 μM | |
| 14 | | 18% @ 1 μM | |
| 15 | | 29 | |
| 16 | | 252 | |
| 17 | | 65 | |
| 18 | | 21 | |
| 19 | | 129 | |
| 20 | | 35 | |
| 21 | | 140 | |
| 22 | | 175 | |
| 23 | | 4.2 | |
| 24 | | 26 | |
| 25 | | 105 | |
| 26 | | 13.7% @ 1 μM | |
| 27 | | 30% @ 1 μM | |
| 28 | | 80 | |
| 29 | | 224 | |

The compounds of the present invention can be administered alone or in combination with one or more therapeutic agents. These include, for example, other anticoagulant, antiplatelet, or platelet inhibitory agents which include non-steroidal anti-inflammatory agents such as aspirin, ibuprofen, naproxen sodium, indomethacin, piroxica, and ticlopidine; thrombin inhibitors such as argatroban, efegatran, inogatran, factor VIIa inhibitors, thrombolytic or fibrinolytic agents such as tissue plasminogen activator, urokinase or streptokinase; GP IIb-IIIa antagonists, and P2Y12 antagonists.

The compounds are thus well suited to formulation for convenient administration to mammals for the prevention and treatment of such disorders.

The following examples further illustrate typical formulations provided by the invention.

### Formulation 1

| Ingredient | Amount | |
|---|---|---|
| compound of Formula I | 0.5 to 800 | mg |
| sodium benzoate | 5 | mg |
| isotonic saline | 1000 | mL |

The above ingredients are mixed and dissolved in the saline for IV administration to a human suffering from, for example, arterial thrombosis.

### Formulation 2

| Ingredient | Amount | |
|---|---|---|
| compound of Formula I | 0.5 to 800 | mg |
| Cellulose, microcrystalline | 400 | mg |
| stearic acid | 5 | mg |
| silicon dioxide | 10 | mg |
| sugar, confectionery | 50 | mg |

The ingredients are blended to uniformity and pressed into a tablet that is well suited for oral administration to a human for preventing, for example, cerebral infarction.

### Formulation 3

| Ingredient | Amount | |
|---|---|---|
| compound of Formula I | 0.5 to 800 | mg |
| starch, dried | 250 | mg |
| magnesium stearate | 10 | mg |

The ingredients are combined and milled to afford material suitable for filling hard gelatin capsules administered to humans suffering from, for example, venous thrombosis.

### Formulation 4

| Ingredient | Amount % wt./(total wt.) |
|---|---|
| compound of Formula I | 1 to 50 |
| Polyethylene glycol 1000 | 32 to 75 |
| Polyethylene glycol 4000 | 16 to 25 |

The ingredients are combined via melting and then poured into molds containing 2.5 g total weight.

While embodiments of the invention have been illustrated and described, it is not intended that these embodiments illustrate and describe all possible forms of the invention. Rather, the words used in the specification are words of description rather than limitation, and it is understood that various changes may be made without departing from the spirit and scope of the invention.

What is claimed is:

1. A compound having Formula I: 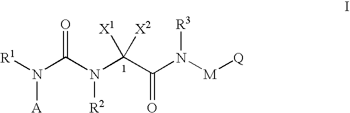
and pharmaceutically acceptable salts thereof, where:
X¹ and X² are hydrogen, alkyl, alkenyl, alkynyl, cycloalkyl, aryl, heteroaryl, aralkyl, cycloalkylalkyl, —(CH₂)ₘ-halogen, —(CH₂)ₘ-heteroaryl, —(CH₂)ₘ—SOR³, —(CH₂)ₘ—OCOR³, —(CH₂)ₘ—OSO₂R³, —(CH₂)ₘ—OSO₂NR⁴R⁵, —(CH₂)ₘ-NR⁶COR³, —(CH₂)ₘ—NR⁶SO₂R³, —(CH₂)ₘ—NR³SO₂NR⁴R⁵, —(CH₂)ₘNR⁴R⁵, —(CH₂)ₘOR³, —CN, —NO₂, —CF₍₃₋ₙ₎Hₙ, —(CH₂)ₘ—O(CH₂)ₘR³ , —(CH₂)ₘ—O(CH₂)ₘ—OR³ , —(CH₂)ₘ—O(CH₂)ₘ—NR⁴R⁵, —(CH₂)ₘR³, —(CH₂)ₘCO₂R³, —(CH₂)ₘCOR³, —(CH₂)ₘCONR⁴R⁵, —(CH₂)ₘNR⁶COR³, —(CH₂)ₘNR⁶CONR⁴R⁵, —(CH₂)ₘSO₂R³, —(CH₂)ₘSO₂NR⁴R⁵, 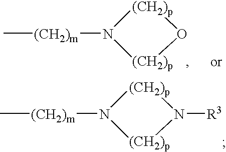
or are joined together to form a substituted or unsubstituted three to eight member ring wherein 0 to 3 atoms of the ring are heteroatoms;
A is aryl, arylcycloalkyl, heteroaryl, heteroarylcycloalkyl, cycloalkyl, or cycloalkenyl;
M is arylene, heteroarylene, cycloalkylene, heterocycloalkylene, cycloalkenylene or heterocycloalkenylene;
Q is —CONR⁴R⁵, aryl, heteroaryl, cycloalkyl, cycloalkenyl, heterocycloalkyl, or heterocycloalkenyl;
R¹ is hydrogen, alkyl, aryl, heteroaryl or alkenyl;
R² is hydrogen, alkyl, aryl, heteroaryl, alkenyl, cycloalkyl, cycloalkylalkyl, aralkyl, heteroaralkyl, heterocycloalkylalkyl, carboxy, —(CH₂)ₘNR⁴R⁵, —(CH₂)ₘOR³, —(CH₂)ₘSR³, —(CH₂)ₘCONR⁴R⁵, or —(CH₂)ₘNR⁶COR³;
R³ is hydrogen, alkyl, aryl, heteroaryl, alkenyl, alkynyl, cycloalkyl, cycloalkylalkyl, aralkyl, or heteroarylalkyl;
R⁶ is hydrogen, alkyl, aryl, heteroaryl, alkenyl, alkynyl, cycloalkyl, cycloalkylalkyl, aralkyl, or heteroarylalkyl;
R⁴ and R⁵ are each independently hydrogen, alkyl, aryl, heteroaryl, alkenyl, alkynyl, cycloalkyl, cycloalkylalkyl, aralkyl, heteroarylalkyl, 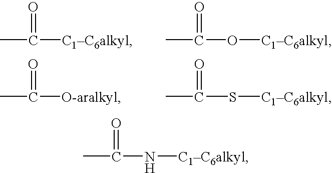
or joined together to form a 3 to 8 member ring;
m is 0 to 8;
n is 0 to 2; and
p is 1 to 3;
with the proviso that when R¹ and R² are H, neither X¹ nor X² is H.

2. The compound of claim 1, wherein A is aryl or heteroaryl.

3. The compound of claim 2, wherein A is 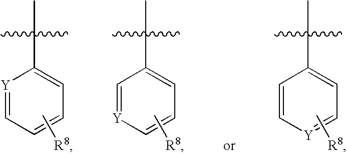
wherein Y is CH or N; and R⁸ is hydrogen, halo, or C₁-C₆ alkyl.

4. The compound of claim 3, wherein A is 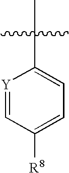
wherein Y is CH or N; and R⁸ is hydrogen, Cl, Br, or F.

5. The compound of claim 4, wherein A is 4-chlorophenyl or 5-chloro-2-pyridyl.

6. The compound of claim 1, wherein M is arylene or heteroarylene.

7. The compound of claim 6, wherein M is 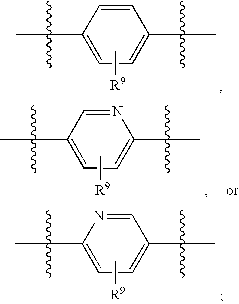
wherein R⁹ is hydrogen, halo, or C₁-C₆ alkyl.

8. The compound of claim 7, wherein M is 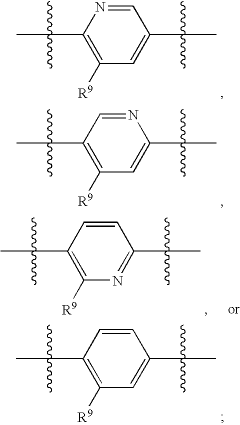
wherein R⁹ is hydrogen, methyl, trifluoromethyl, Cl, Br, or F.

9. The compound of claim 8, wherein M is phenylene-1,4-diyl, 2-fluoro-phenylene-1,4-diyl, 2-methyl-phenylene-1,4-diyl, 2-trifluoromethyl-phenylene-1,4-diyl, or pyridine-2,5-diyl.

10. The compound of claim 1, wherein Q is aryl, heteroaryl or heterocycloalkyl.

11. The compound of claim 10, wherein Q is 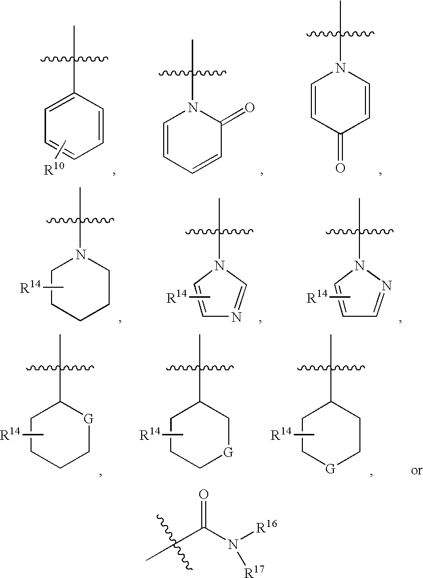
wherein G is O, S, NH, N—(C₁-C₆alkyl), 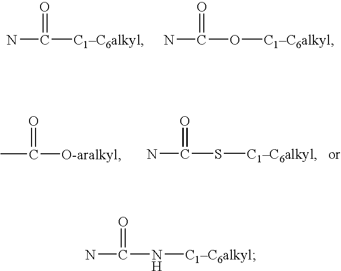
R¹⁴ is hydrogen, halo, C₁-C₆ alkyl, —SO₂NR¹²R¹³, —SO₂alkyl or oxo; R¹⁶ and R¹⁷ are independently hydrogen, C₁-C₆ alkyl, or are joined together to form a saturated or unsaturated 3 to 8 membered ring; and R¹⁰ is hydrogen, halo, C₁-C₆ alkyl, —SO₂NR¹²R¹³, or —SO₂alkyl, C₁-C₆ alkyl, wherein R¹² and R¹³ are independently hydrogen, C₁-C₆ alkyl, or are joined together to form a saturated 5 to 7 membered ring.

12. The compound of claim 11, wherein Q is 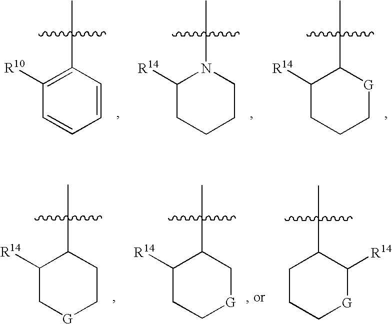
wherein G is NH, N—(C₁-C₆alkyl) or 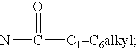
R¹⁴ is hydrogen, —SO₂NR¹²R¹³, —SO₂alkyl or oxo; and R¹⁰ is hydrogen, Cl, Br, F, —SO₂NR¹²R¹³, or —SO₂alkyl, wherein R¹² and R¹³ are independently hydrogen, or C₁-C₆ alkyl.

13. The compound of claim 11, wherein Q is 2-methanesulfonylphenyl, 2-sulfamoylphenyl, 2-oxo-2H-pyridin-1-yl, or 2-oxo-piperidin-1-yl.

14. The compound of claim 1, wherein X¹ and X² are hydrogen, alkyl, —(CH₂)ₘOR³, or alkenyl.

15. The compound of claim 1, wherein X¹ and X² are alkyl, —(CH₂)ₘOR³, alkenyl or —CH₂—NR⁷R^{7′} where R⁷ and R^{7′} are independently hydrogen, —C₁-C₆ alkyl

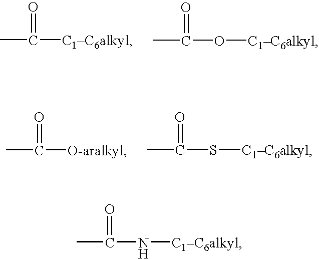

16. The compound of claim 1, wherein X¹ and X² are hydrogen, methyl, —CH₂—OH, —CH₂—NH₂, —CH₂—N(CH₃)₂, or —CH₂—N(CH₂CH₃)₂.

17. The compound of claim 1, wherein X¹ and X² together form a cyclopropyl, cyclobutyl, cyclopentyl, cyclohexyl, or cyclopentenyl ring.

18. The compound of claim 1, wherein X¹ and X²together are 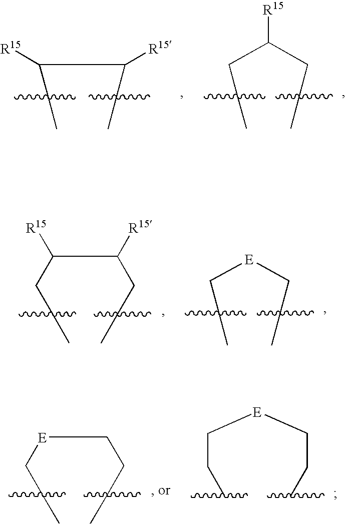
wherein R¹⁵ and R^{15′} are independently hydrogen, —(CH₂)₁₋₆—OH, —(CH₂)₁₋₆—O—C₁-C₆ alkyl, —(CH₂)₁₋₆—NH₂, —COOH, or —OH; and E is O, S, or NR¹⁶ wherein R¹⁶ is hydrogen, —C₁-C₆ alkyl,

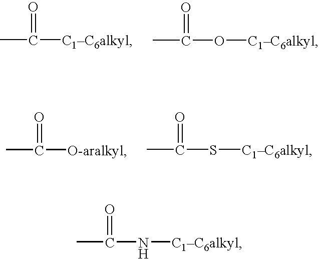

19. The compound of claim 1, wherein R² is alkyl, aryl, heteroaryl, cycloalkyl, cycloalkylalkyl, aralkyl, heteroaralkyl, heterocycloalkylalkyl, carboxy, —(CH₂)ₘNR⁴R⁵, —(CH₂)ₘOR³, —(CH₂)ₘSR³, —(CH₂)ₘCONR⁴R⁵, or —(CH₂)ₘNR⁶COR³; wherein R³, R⁴, R⁵ and R⁶ as as described in claim 1.

20. The compound of claim 19, wherein R² is C₁-C₆ alkyl, phenyl, pyridyl, cyclopropyl, cyclopropylmethyl, cyclobutylmethyl, cyclopentylmethyl, cyclohexylmethyl, 2-cyclopropylethyl, 2-cyclopentylethyl, benzyl, 2-pyridinylmethyl, 3-pyridinylmethyl, 4-pyridinylmethyl, 3-(2-pyridinyl)-propyl, thienylmethyl, 2-morpholin-4-yl-ethyl, 2-thiomorpholin-4-yl-ethyl, —(CH₂)₁₋₃NH₂, —(CH₂)₁₋₃N(C₁-C₆alkyl)₂, —(CH₂)₁₋₃NHC₁-C₆alkyl, —(CH₂)₁₋₃OC₁-C₆alkyl, —(CH₂)₁₋₃SC1-C₆alkyl, —(CH₂)₁₋₃CONH₂, —(CH₂)₁₋₃CON(C₁-C₆alkyl)₂, —(CH₂)₁₋₃CONHC₁-C₆alkyl, or —(CH₂)₁₋₃NHCOC₁-C₆alkyl.

21. The compound of claim 1, wherein where A is 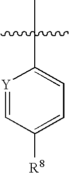
wherein Y is CH or N; and R⁸ is hydrogen, Cl, Br, or F;
M is 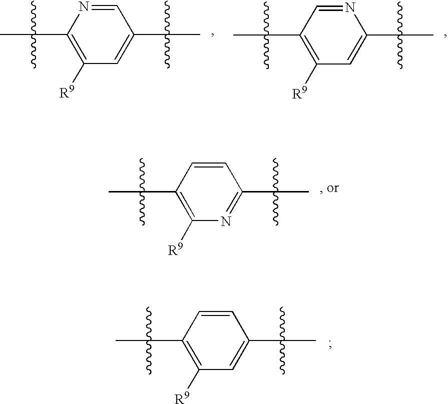
wherein R⁹ is hydrogen, Cl, Br, or F;
Q is 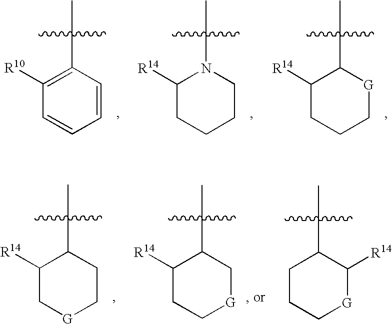
wherein G is NH, N—(C₁-C₆alkyl) or 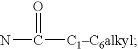
R¹⁴ is hydrogen, —SO₂NR¹²R¹³, —SO₂alkyl or oxo; and R¹⁰ is hydrogen, Cl, Br, F, —SO₂NR¹²R¹³, or —SO₂alkyl, where R¹² and R¹³ are independently hydrogen, or C₁-C₆ alkyl;
X¹ and X₂ are hydrogen, methyl, —CH₂—OH, —CH₂—NR⁷R^{7′} where R⁷ and R^{7′} are independently hydrogen or C₁-C₆ alkyl, or X¹ and X₂ together form a cyclopropyl, cyclobutyl, cyclopentyl, cyclohexyl, or cyclopentenyl ring or together are 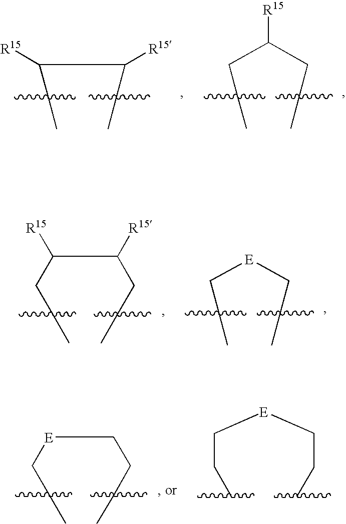
wherein R¹⁵ and R^{15′} are independently hydrogen, —(CH₂)₁₋₆—OH, —(CH₂)₁₋₆—O—C₁-C₆ alkyl, —(CH₂)₁₋₆—NH₂, —COOH, or —OH; and E is O, S, or NR¹⁶ where R¹⁶ is R¹⁶ is hydrogen, C₁-C₆ alkyl, 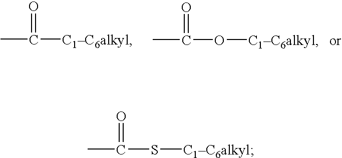
R¹ and R³ are each independently hydrogen, or C₁-C₆alkyl; and
R² is hydrogen, C₁-C₆ alkyl, phenyl, pyridyl, cyclopropyl, cyclopropylmethyl, cyclobutylmethyl, cyclopentylmethyl, cyclohexylmethyl, 2-cyclopropylethyl, 2-cyclopentylethyl, benzyl, 2-pyridinylmethyl, 3-pyridinylmethyl, 4-pyridinylmethyl, 3-(2-pyridinyl)-propyl, thienylmethyl, 2-morpholin-4-yl-ethyl, 2-thiomorpholin-4-yl-ethyl, —(CH₂)₁₋₃NH₂, —(CH₂)₁₋₃N(C₁-C₆alkyl)₂, —(CH₂)₁₋₃NHC₁-C₆alkyl, —(CH₂)₁₋₃OC₁-C₆alkyl, —(CH₂)₁₋₃SC1-C₆alkyl, —(CH₂)₁₋₃CONH₂, —(CH₂)₁₋₃CON(C₁-C₆alkyl)₂, —(CH₂)₁₋₃CONHC₁-C₆alkyl, or —(CH₂)₁₋₃NHCOC₁-C₆alkyl.

22. The compound of claim 1, wherein the compounds is
1-[3-(4-Chloro-phenyl)-ureido)-cyclopropanecarboxylic acid (3-fluoro-2′-methanesulfonyl-biphenyl-4-yl)-amide;
1-[3-(5-Chloro-pyridin-2-yl)-ureido]-cyclopropanecarboxylic acid (3-fluoro-2′-sulfamoyl-biphenyl-4-yl)-amide;
2-[3-(5-Chloro-pyridin-2-yl)-ureido]-N-(3-fluoro-2′-methanesulfonyl-biphenyl-4-yl)-2-methyl-propionamide;
2-[3-(4-Chloro-phenyl)-ureido]-N-(3-fluoro-2′-sulfamoyl-biphenyl-4-yl)-2-methyl-propionamide;
4-[3-(4-Chloro-phenyl)-ureido]-tetrahydro-thiopyran-4-carboxylic acid (3-fluoro-2′-sulfamoyl-biphenyl-4-yl)-amide;
1-[3-(4-Chloro-phenyl)-ureido]-cyclopropanecarboxylic acid (3-fluoro-2′-sulfamoyl-biphenyl-4-yl)-amide;
1-[3-(5-Chloro-pyridin-2-yl)-ureido]-cyclopropanecarboxylic acid (3-fluoro-2′-methanesulfonyl-biphenyl-4-yl)-amide;
4-[3-(4-Chloro-phenyl)-ureido]-tetrahydro-pyran-4-carboxylic acid (3-fluoro-2′-sulfamoyl-biphenyl-4-yl)-amide;
1-[3-(4-Chloro-phenyl)-ureido]-cyclopentanecarboxylic acid (2′-methanesulfonyl-biphenyl-4-yl)-amide;
1-[3-(4-Chloro-phenyl)-ureido]-cyclohexanecarboxylic acid (2′-methanesulfonyl-biphenyl-4-yl)-amide;
2-[3-(4-Chloro-phenyl)-1-methyl-ureido]-N-(3-fluoro-2′-methanesulfonyl-biphenyl-4-yl)-acetamide;
2-[3-(4-Chloro-phenyl)-1,3-dimethyl-ureido]-N-(3-fluoro-2′-methanesulfonyl-biphenyl-4-yl)-acetamide;
2-[3-(4-Chloro-phenyl)-ureido]-3-hydroxy-2-hydroxymethyl-N-(2′-sulfamoyl-biphenyl-4-yl)-propionamide;
1-[3-(4-Chloro-phenyl)-ureido]-cyclopropanecarboxylic acid (2′-methanesulfonyl-biphenyl-4-yl)-amide;
2-[3-(4-Chloro-phenyl)-ureido]-N-(3-fluoro-2′-methanesulfonyl-biphenyl-4-yl)-2-methyl-propionamide;
2-[3-(4-Chloro-phenyl)-ureido]-N-(3-fluoro-2′-methanesulfonyl-biphenyl-4-yl)-acetamide;
1-[3-(4-Chloro-phenyl)-ureido]-cyclopent-3-enecarboxylic acid (3-fluoro-2′-sulfamoyl-biphenyl-4-yl)-amide; and
2-[3-(4-Chloro-phenyl)-3-methyl-ureido]-N-(3-fluoro-2′-methanesulfonyl-biphenyl-4-yl)-acetamide;
(1S,2S)-1-[3-(4-Chloro-phenyl)-ureido]-2-hydroxymethyl-cyclopropanecarboxylic acid (3-fluoro-2′-methanesulfonyl-biphenyl-4-yl)-amide;
(1R,2S)-1-[3-(4-Chloro-phenyl)-ureido]-2-hydroxymethyl-cyclopropanecarboxylic acid (3-fluoro-2′-methanesulfonyl-biphenyl-4-yl)-amide;
(1R,2S)-1-[3-(4-Chloro-phenyl)-ureido]-2-hydroxymethyl-cyclopropanecarboxylic acid [2-fluoro-4-(2-oxo-piperidin-1-yl)-phenyl]-amide;
(1S,2S)-1-[3-(4-Chloro-phenyl)-ureido]-2-hydroxymethyl-cyclopropanecarboxylic acid [2-fluoro-4-(2-oxo-piperidin-1-yl)-phenyl]-amide;
2-[3-(4-Chloro-phenyl)-ureido]-N-[2-fluoro-4-(2-oxo-piperidin-1-yl)-phenyl]-2-methyl-propionamide;
2-[3-(5-Chloro-pyridin-2-yl)-ureido]-2-methyl-N-[4-(2-oxo-piperidin-1-yl)-phenyl]-propionamide;
2-[3-(4-Chloro-phenyl)-ureido]-2-methyl-N-[4-(2-oxo-piperidin-1-yl)-phenyl]-propionamide;
2-[3-(5-Chloro-pyridin-2-yl)-ureido]-N-[2-fluoro-4-(2-oxo-piperidin-1-yl)-phenyl]-2-methyl-propion amide;
N-[2-Fluoro-4-(2-oxo-piperidin-1-yl)-phenyl]-2-[3-(4-fluoro-phenyl)-ureido]-2-methyl-propionamide;
1-[3-(4-Chloro-phenyl)-ureido]-cyclopropanecarboxylic acid [2-fluoro-4-(2-oxo-piperidin-1-yl)-phenyl]-amide;
1-[3-(5-Chloro-pyridin-2-yl)-ureido]-cyclopropanecarboxylic acid [4-(2-oxo-piperidin-1-yl)-phenyl]-amide;
1-[3-(4-Chloro-phenyl)-ureido]-cyclopropanecarboxylic acid [4-(2-oxo-piperidin-1-yl)-phenyl]-amide;
1-[3-(5-Chloro-pyridin-2-yl)-ureido]-cyclopropanecarboxylic acid [2-fluoro-4-(2-oxo-piperidin-1-yl)-phenyl]-amide;
1-[3-(4-Fluoro-phenyl)-ureido]-cyclopropanecarboxylic acid [2-fluoro-4-(2-oxo-piperidin-1-yl)-phenyl]-amide;
1-[3-(4-Chloro-phenyl)-ureido]-cyclohexanecarboxylic acid [2-fluoro-4-(2-oxo-piperidin-1-yl)-phenyl]-amide;
1-[3-(5-Chloro-pyridin-2-yl)-ureido]-cyclohexanecarboxylic acid [4-(2-oxo-piperidin-1-yl)-phenyl]-amide;
1-[3-(4-Chloro-phenyl)-ureido]-cyclohexanecarboxylic acid [4-(2-oxo-piperidin-1-yl)-phenyl]-amide;
1-[3-(5-Chloro-pyridin-2-yl)-ureido]-cyclohexanecarboxylic acid [2-fluoro-4-(2-oxo-piperidin-1-yl)-phenyl]-amide;
1-[3-(4-Fluoro-phenyl)-ureido]-cyclohexanecarboxylic acid [2-fluoro-4-(2-oxo-piperidin-1-yl)-phenyl]-amide;
2-[3-(4-Chloro-phenyl)-ureido]-N-[2-fluoro-4-(2-oxo-piperidin-1-yl)-phenyl]-3-hydroxy-2-hydroxymethyl-propionamide;
2-[3-(5-Chloro-pyridin-2-yl)-ureido]-3-hydroxy-2-hydroxymethyl-N-[4-(2-oxo-piperidin-1-yl)-phenyl]-propionamide;
2-[3-(4-Chloro-phenyl)-ureido]-3-hydroxy-2-hydroxymethyl-N-[4-(2-oxo-piperidin-1-yl)-phenyl]-propionamide;
2-[3-(5-Chloro-pyridin-2-yl)-ureido]-N-[2-fluoro-4-(2-oxo-piperidin-1-yl)-phenyl]-3-hydroxy-2-hydroxymethyl-propionamide;
N-[2-Fluoro-4-(2-oxo-piperidin-1-yl)-phenyl]-2-[3-(4-fluoro-phenyl)-ureido]-3-hydroxy-2-hydroxymethyl-propionamide;
2-[3-(4-Chloro-phenyl)-ureido]-N-[2-fluoro-4-(2-oxo-piperidin-1-yl)-phenyl]-acetamide;
2-[3-(5-Chloro-pyridin-2-yl)-ureido]-N-[2-fluoro-4-(2-oxo-piperidin-1-yl)-phenyl]-acetamide;
2-[3-(5-Chloro-pyridin-2-yl)-ureido]-N-[4-(2-oxo-piperidin-1-yl)-phenyl]-acetamide;
2-[3-(4-Chloro-phenyl)-ureido]-N-[4-(2-oxo-piperidin-1-yl)-phenyl]-acetamide;
1-[3-(4-Chloro-phenyl)-ureido]-cyclopropanecarboxylic acid [5-(2-methanesulfonyl-phenyl)-pyridin-2-yl]-amide;
1-[3-(4-Chloro-phenyl)-ureido]-cyclopropanecarboxylic acid [5-(2-sulfamoyl-phenyl)-pyridin-2-yl]-amide;
1-[3-(5-Chloro-pyridin-2-yl)-ureido]-cyclopropanecarboxylic acid [5-(2-methanesulfonyl-phenyl)-pyridin-2-yl]-amide;
1-[3-(5-Chloro-pyridin-2-yl)-ureido]-cyclopropanecarboxylic acid [5-(2-sulfamoyl-phenyl)-pyridin-2-yl]-amide;
1-[3-(4-Chloro-phenyl)-ureido]-cyclopropanecarboxylic acid (2′-methanesulfonyl-3-trifluoromethyl-biphenyl-4-yl)-amide;
1-[3-(4-Chloro-phenyl)-ureido]-cyclopropanecarboxylic acid (2′-sulfamoyl-3-trifluoromethyl-biphenyl-4-yl)-amide;
1-[3-(5-Chloro-pyridin-2-yl)-ureido]-cyclopropanecarboxylic acid (2′-methanesulfonyl-3-trifluoromethyl-biphenyl-4-yl)-amide;
1-[3-(5-Chloro-pyridin-2-yl)-ureido)-cyclopropanecarboxylic acid (2′-sulfamoyl-3-trifluoromethyl-biphenyl-4-yl)-amide;
1-[3-(4-Chloro-phenyl)-ureido]-cyclopropanecarboxylic acid (2′-methanesulfonyl-3-methyl-biphenyl-4-yl)-amide;
1-[3-(4-Chloro-phenyl)-ureido]-cyclopropanecarboxylic acid (3-methyl-2′-sulfamoyl-biphenyl-4-yl)-amide;
1-[3-(5-Chloro-pyridin-2-yl)-ureido]-cyclopropanecarboxylic acid (2′-methanesulfonyl-3-methyl-biphenyl-4-yl)-amide;
1-[3-(5-Chloro-pyridin-2-yl)-ureido]-cyclopropanecarboxylic acid (3-methyl-2′-sulfamoyl-biphenyl-4-yl)-amide;
2-[3-(5-Chloro-pyridin-2-yl)-1-methyl-ureido]-N-(2′-methanesulfonyl-biphenyl-4-yl)-acetamide;
2-[3-(4-Chloro-phenyl)-1-methyl-ureido]-N-(2′-sulfamoyl-biphenyl-4-yl)-acetamide;
2-[3-(5-Chloro-pyridin-2-yl)-1-methyl-ureido]-N-(3-fluoro-2′-sulfamoyl-biphenyl-4-yl)-acetamide;
2-[3-(4-Chloro-phenyl)-1-methyl-ureido]-N-(3-fluoro-2′-sulfamoyl-biphenyl-4-yl)-acetamide;
2-[3-(5-Chloro-pyridin-2-yl)-1-methyl-ureido]-N-(3-fluoro-2′-methanesulfonyl-biphenyl-4-yl)-acetamide;
2-[3-(4-Chloro-phenyl)-1-methyl-ureido]-N-(2′-methanesulfonyl-biphenyl-4-yl)-acetamide;
1-[3-(4-Chloro-phenyl)-ureido]-2-hydroxymethyl-cyclopropanecarboxylic acid (2′-methanesulfonyl-biphenyl-4-yl)-amide;
1-[3-(4-Chloro-phenyl)-ureido]-2-hydroxymethyl-cyclopropanecarboxylic acid (2′-sulfamoyl-biphenyl-4-yl)-amide;
1-[3-(4-Chloro-phenyl)-ureido]-2-hydroxymethyl-cyclopropanecarboxylic acid (3-fluoro-2′-methanesulfonyl-biphenyl-4-yl)-amide;
1-[3-(4-Chloro-phenyl)-ureido]-2-hydroxymethyl-cyclopropanecarboxylic acid (3-fluoro-2′-sulfamoyl-biphenyl-4-yl)-amide;
3-[3-(4-Chloro-phenyl)-ureido]-pyrrolidine-3-carboxylic acid (3-fluoro-2′-methanesulfonyl-biphenyl-4-yl)-amide;
3-[3-(4-Chloro-phenyl)-ureido]-pyrrolidine-3-carboxylic acid (3-fluoro-2′-sulfamoyl-biphenyl-4-yl)-amide;
1-[3-(4-Chloro-phenyl)-ureido]-3-hydroxymethyl-cyclobutanecarboxylic acid (3-fluoro-2′-methanesulfonyl-biphenyl-4-yl)-amide;
1-[3-(4-Chloro-phenyl)-ureido]-3-hydroxymethyl-cyclobutanecarboxylic acid (3-fluoro-2′-sulfamoyl-biphenyl-4-yl)-amide;
1-[3-(4-Chloro-phenyl)-ureido]-2-methoxymethyl-cyclopropanecarboxylic acid (3-fluoro-2′-methanesulfonyl-biphenyl-4-yl)-amide;
1-[3-(4-Chloro-phenyl)-ureido]-2-methoxymethyl-cyclopropanecarboxylic acid (3-fluoro-2′-sulfamoyl-biphenyl-4-yl)-amide;
2-Aminomethyl-1-[3-(4-chloro-phenyl)-ureido]-cyclopropanecarboxylic acid (3-fluoro-2′-methanesulfonyl-biphenyl-4-yl)-amide;
2-Aminomethyl-1-[3-(4-chloro-phenyl)-ureido]-cyclopropanecarboxylic acid (3-fluoro-2′-sulfamoyl-biphenyl-4-yl)-amide;
2-[3-(4-Chloro-phenyl)-ureido]-2-(3-fluoro-2′-methanesulfonyl-biphenyl-4-ylcarbamoyl)-cyclopropanecarboxylic acid;
2-[3-(4-Chloro-phenyl)-ureido]-2-(3-fluoro-2′-sulfamoyl-biphenyl-4-ylcarbamoyl)-cyclopropanecarboxylic acid;
3-[3-(4-Chloro-phenyl)-ureido]-1-methyl-pyrrolidine-3-carboxylic acid (3-fluoro-2′-methanesulfonyl-biphenyl-4-yl)-amide;
3-[3-(4-Chloro-phenyl)-ureido]-1-methyl-pyrrolidine-3-carboxylic acid (3-fluoro-2′-sulfamoyl-biphenyl-4-yl)-amide;
1-Acetyl-3-[3-(4-chloro-phenyl)-ureido]-pyrrolidine-3-carboxylic acid (3-fluoro-2′-methanesulfonyl-biphenyl-4-yl)-amide;
1-Acetyl-3-[3-(4-chloro-phenyl)-ureido]-pyrrolidine-3-carboxylic acid (3-fluoro-2′-sulfamoyl-biphenyl-4-yl)-amide;
1-[3-(4-Chloro-phenyl)-ureido]-3-methoxymethyl-cyclobutanecarboxylic acid (3-fluoro-2′-methanesulfonyl-biphenyl-4-yl)-amide;
1-[3-(4-Chloro-phenyl)-ureido]-3-methoxymethyl-cyclobutanecarboxylic acid (3-fluoro-2′-sulfamoyl-biphenyl-4-yl)-amide;
3-Aminomethyl-1-[3-(4-chloro-phenyl)-ureido]-cyclobutanecarboxylic acid (3-fluoro-2′-methanesulfonyl-biphenyl-4-yl)-amide;
3-Aminomethyl-1-[3-(4-chloro-phenyl)-ureido]-cyclobutanecarboxylic acid (3-fluoro-2′-sulfamoyl-biphenyl-4-yl)-amide;
3-[3-(4-Chloro-phenyl)-ureido]-3-(3-fluoro-2′-methanesulfonyl-biphenyl-4-ylcarbamoyl)-cyclobutanecarboxylic acid;
3-[3-(4-Chloro-phenyl)-ureido]-3-(3-fluoro-2′-sulfamoyl-biphenyl-4-ylcarbamoyl)-cyclobutanecarboxylic acid;
4-[3-(4-Chloro-phenyl)-ureido]-piperidine-4-carboxylic acid (3-fluoro-2′-methanesulfonyl-biphenyl-4-yl)-amide;
4-[3-(4-Chloro-phenyl)-ureido]-piperidine-4-carboxylic acid (3-fluoro-2′-sulfamoyl-biphenyl-4-yl)-amide;
4-[3-(4-Chloro-phenyl)-ureido]-1-methyl-piperidine-4-carboxylic acid (3-fluoro-2′-methanesulfonyl-biphenyl-4-yl)-amide;
4-[3-(4-Chloro-phenyl)-ureido]-1-methyl-piperidine4-carboxylic acid (3-fluoro-2′-sulfamoyl-biphenyl-4-yl)-amide;
1-Acetyl-4-[3-(4-chloro-phenyl)-ureido]-piperidine-4-carboxylic acid (3-fluoro-2′-methanesulfonyl-biphenyl-4-yl)-amide;
1-Acetyl-4-[3-(4-chloro-phenyl)-ureido]-piperidine-4-carboxylic acid (3-fluoro-2′-sulfamoyl-biphenyl-4-yl)-amide;
1-[3-(4-Chloro-phenyl)-ureido]-3,4-dihydroxy-cyclopentanecarboxylic acid (3-fluoro-2′-methanesulfonyl-biphenyl-4-yl)-amide;
1-[3-(4-Chloro-phenyl)-ureido]-3,4-dihydroxy-cyclopentanecarboxylic acid (3-fluoro-2′-sulfamoyl-biphenyl-4-yl)-amide;
3-[3-(4-Chloro-phenyl)-ureido)-tetrahydro-furan-3-carboxylic acid (3-fluoro-2′-methanesulfonyl-biphenyl-4-yl)-amide;
3-[3-(4-Chloro-phenyl)-ureido]-tetrahydro-furan-3-carboxylic acid (3-fluoro-2′-sulfamoyl-biphenyl-4-yl)-amide;
3-[3-(4-Chloro-phenyl)-ureido]-tetrahydro-thiophene-3-carboxylic acid (3-fluoro-2′-methanesulfonyl-biphenyl-4-yl)-amide;
3-[3-(4-Chloro-phenyl)-ureido]-1-methyl-pyrrolidine-3-carboxylic acid [2-fluoro-4-(2-oxo-piperidin-1-yl)-phenyl]-amide;
1-Acetyl-3-[3-(4-chloro-phenyl)-ureido]-pyrrolidine-3-carboxylic acid [2-fluoro-4-(2-oxo-piperidin-1-yl)-phenyl -amide;
1-Acetyl-3-[3-(4-chloro-phenyl)-ureido]-azetidine-3-carboxylic acid (3-fluoro-2′-methanesulfonyl-biphenyl-4-yl)-amide;
1-Acetyl-3-[3-(4-chloro-phenyl)-ureido]-azetidine-3-carboxylic acid (3-fluoro-2′-sulfamoyl-biphenyl-4-yl)-amide;
1-[3-(4-Chloro-phenyl)-1-methyl-ureido]-2-hydroxymethyl-cyclopropanecarboxylic acid [2-fluoro-4-(2-oxo-piperidin-1-yl)-phenyl]-amide;
1-[3-(4-Chloro-phenyl)-1-methyl-ureido]-2-hydroxymethyl-cyclopropanecarboxylic acid (3-fluoro-2′-sulfamoyl-biphenyl-4-yl)-amide;
1-[3-(4-Chloro-phenyl)-1-methyl-ureido]-2-hydroxymethyl-cyclopropanecarboxylic acid (3-fluoro-2′-methanesulfonyl-biphenyl-4-yl)-amide;
3-[3-(4-Chloro-phenyl)-ureido]-tetrahydro-thiophene-3-carboxylic acid (3-fluoro-2′-sulfamoyl-biphenyl-4-yl)-amide;
3-[3-(4-Chloro-phenyl)-ureido]-1-methyl-azetidine-3-carboxylic acid (3-fluoro-2′-methanesulfonyl-biphenyl-4-yl)-amide;
3-[3-(4-Chloro-phenyl)-ureido]-1-methyl-azetidine-3-carboxylic acid (3-fluoro-2′-sulfamoyl-biphenyl-4-yl)-amide;
1-[3-(4-Chloro-phenyl)-ureido]-2-hydroxymethyl-cyclopropanecarboxylic acid [2-fluoro-4-(2-oxo-piperidin-1-yl)-phenyl]-amide;
1-[3-(4-Chloro-phenyl)-ureido]-2-methoxymethyl-cyclopropanecarboxylic acid [2-fluoro-4-(2-oxo-piperidin-1-yl)-phenyl]-amide;
3-Amino-2-aminomethyl-2-[3-(4-chloro-phenyl)-ureido]-N-(3-fluoro-2′-sulfamoyl-biphenyl-4-yl)-propionamide;
3-Amino-2-aminomethyl-2-[3-(4-chloro-phenyl)-ureido]-N-(3-fluoro-2′-methanesulfonyl-biphenyl-4-yl)-propionamide;
2-[3-(4-Chloro-phenyl)-ureido]-3-ethylamino-2-ethylaminomethyl-N-(3-fluoro-2′-methanesulfonyl-biphenyl-4-yl)-propionamide;
2-[3-(4-Chloro-phenyl)-1-cyclopropylmethyl-ureido]-N-(3-fluoro-2′-sulfamoyl-biphenyl-4-yl)-acetamide;
2-[3-(4-Chloro-phenyl)-1-cyclopropylmethyl-ureido]-N-[2-fluoro-4-(2-oxo-piperidin-1-yl)-phenyl]-acetaminde;
2-[3-(4-Chloro-phenyl)-1-cyclopropylmethyl-ureido]-N-[2-fluoro-4-(2-oxo-2H-pyridin-1-yl)-phenyl]-acetamide;
2-[3-(5-Chloro-pyridin-2-yl)-1-cyclopropylmethyl-ureido]-N-(3-fluoro-2′-methanesulfonyl-biphenyl-4-yl)-acetamide;
2-[3-(4-Chloro-phenyl)-1-cyclopropyl-ureido]-N-(3-fluoro-2′-methanesulfonyl-biphenyl-4-yl)-acetamide;
2-[3-(5-Chloro-pyridin-2-yl)-1-cyclopropylmethyl-ureido]-N-(3-fluoro-2′-sulfamoyl-biphenyl-4-yl)-acetamide;
2-[3-(5-Chloro-pyridin-2-yl)-1-cyclopropylmethyl-ureido]-N-[2-fluoro-4-(2-oxo-piperidin-1-yl)-phenyl]-acetamide;
2-[3-(5-Chloro-pyridin-2-yl)-1-cyclopropylmethyl-ureido]-N-[2-fluoro-4-(2-oxo-2H-pyridin-1-yl)-phenyl]-acetamide;
2-[3-(4-Chloro-phenyl)-1-isopropyl-ureido]-N-(3-fluoro-2′-methanesulfonyl-biphenyl-4-yl)-acetamide;
2-[3-(4-Chloro-phenyl)-1-cyclopentyl-ureido]-N-(3-fluoro-2′-methanesulfonyl-biphenyl-4-yl)-acetamide;
2-[3-(4-Chloro-phenyl)-1-cyclopentylmethyl-ureido]-N-(3-fluoro-2′-methanesulfonyl-biphenyl-4-yl)-acetamide;
2-[3-(4-Chloro-phenyl)-1-(2-cyclopropyl-ethyl)-ureido]-N-(3-fluoro-2′-methanesulfonyl-biphenyl-4-yl)-acetamide;
2-[3-(4-Chloro-phenyl)-1-phenyl-ureido]-N-(3-fluoro-2′-methanesulfonyl-biphenyl-4-yl)-acetamide;
2-[3-(4-Chloro-phenyl)-1-thiophen-3-ylmethyl-ureido]-N-(3-fluoro-2′-methanesulfonyl-biphenyl-4-yl)-acetamide;
2-[3-(4-Chloro-phenyl)-1-pyridin-3-ylmethyl-ureido]-N-(3-fluoro-2′-methanesulfonyl-biphenyl-4-yl)-acetamide;
2-[3-(4-Chloro-phenyl)-1-cyclohexylmethyl-ureido]-N-(3-fluoro-2′-methanesulfonyl-biphenyl-4-yl)-acetamide;
2-[3-(4-Chloro-phenyl)-1-(2-cyclopentyl-ethyl)-ureido]-N-(3-fluoro-2′-methanesulfonyl-biphenyl-4-yl)-acetamide;
2-[3-(4-Chloro-phenyl)-1-thiophen-2-ylmethyl-ureido) -N-(3-fluoro-2′-methanesulfonyl-biphenyl-4-yl)-acetamide;
2-[3-(4-Chloro-phenyl)-1-pyridin-2-ylmethyl-ureido)-N-(3-fluoro-2′-methanesulfonyl-biphenyl-4-yl)-acetamide;
2-[3-(4-Chloro-phenyl)-1-pyridin-4-ylmethyl-ureido]-N-(3-fluoro-2′-methanesulfonyl-biphenyl-4-yl)-acetamide;
2-[3-(4-Chloro-phenyl)-1-(2-ethoxy-ethyl)-ureido]-N-(3-fluoro-2′-methanesulfonyl-biphenyl-4-yl)-acetamide;
2-[3-(4-Chloro-phenyl)-1-(2-methylsulfanyl-ethyl)-ureido]-N-(3-fluoro-2′-methanesulfonyl-biphenyl-4-yl)-acetamide;
{3-(4-Chloro-phenyl)-1-[(3-fluoro-2′-methanesulfonyl-biphenyl-4-ylcarbamoyl)-methyl]-ureido}-acetic acid;
2-[3-(4-Chloro-phenyl)-1-(2-morpholin-4-yl-ethyl)-ureido]-N-(3-fluoro-2′-methanesulfonyl-biphenyl-4-yl)-acetamide;
2-[3-(4-Chloro-phenyl)-1-(2-thiomorpholin-4-yl-ethyl)-ureido]-N-(3-fluoro-2′-methanesulfonyl-biphenyl-4-yl)-acetamide;
2-[3-(4-Chloro-phenyl)-l1-phenethyl-ureido]-N-(3-fluoro-2′-methanesulfonyl-biphenyl-4-yl)-acetamide;
2-[3-(4-Chloro-phenyl)-1-(2-methylsulfanyl-ethyl)-ureido]-N-(3-fluoro-2′-methanesulfonyl-biphenyl-4-yl)-acetamide;
2-[3-(4-Chloro-phenyl)-1-methylcarbamoylmethyl-ureido]-N-(3-fluoro-2′-methanesulfonyl-biphenyl-4-yl)-acetamide;
2-{3-(4-Chloro-phenyl)-1-[2-(4-methyl-piperazin-1-yl)-ethyl]-ureido}-N-(3-fluoro-2′-methanesulfonyl-biphenyl-4-yl)-acetamide;
2-[1-(2-Acetylamino-ethyl)-3-(4-chloro-phenyl)-ureido]-N-(3-fluoro-2′-methanesulfonyl-biphenyl-4-yl)-acetamide;
2-[3-(4-Chloro-phenyl)-1-(2,2-dimethyl-propyl)-ureido]-N-(3-fluoro-2′-methanesulfonyl-biphenyl-4-yl)-acetamide;
3-[3-(4-Chloro-phenyl)-ureido]-3-(3-fluoro-2′-methanesulfonyl-biphenyl-4-ylcarbamoyl)-pyrrolidine-1-carboxylic acid benzyl ester;
2-[3-(4-Chloro-phenyl)-1-(2,2-dimethyl-propyl)-ureido]-N-[2-fluoro-4-(2-oxo-piperidin-1-yl)-phenyl]-acetamide;
2-[3-(4-Chloro-phenyl)-1-cyclobutylmethyl-ureido]-N-(3-fluoro-2′-methanesulfonyl-biphenyl-4-yl)-acetamide;
2-[3-(4-Chloro-phenyl)-1-cyclopropylmethyl-ureido]-N-(3-fluoro-2′-methanesulfonyl-biphenyl-4-yl)-acetamide;
2-[3-(4-Chloro-phenyl)-1-(2-methoxy-ethyl)-ureido]-N-(3-fluoro-2′-methanesulfonyl-biphenyl-4-yl)-acetamide;
2-[3-(4-Chloro-phenyl)-1-isobutyl-ureido]-N-(3-fluoro-2′-methanesulfonyl-biphenyl-4-yl)-acetamide;
2-[3-(4-Chloro-phenyl)-1-(2-dimethylamino-ethyl)-ureido]-N-(3-fluoro-2′-methanesulfonyl-biphenyl-4-yl)-acetamide;
2-[1-Benzyl-3-(4-chloro-phenyl)-ureido]-N-(3-fluoro-2′methanesulfonyl-biphenyl-4-yl)-acetamide;
2-[3-(4-Chloro-phenyl)-1-(4-methoxy-benzyl) ureido)-N-(3-fluoro-2′-methanesulfonyl-biphenyl-4-yl)-acetamide;
(1R,2S)-(1-[3-(4-Chloro-phenyl)-ureido]-2-hydroxymethyl-cyclopropanecarboxylic acid [2-fluoro4-(2-oxo-2H-pyridin-1-yl)-phenyl]-amide;
(1S,2S)-1-[3-(4-Chloro-phenyl)-ureido]-2-hydroxymethyl-cyclopropanecarboxylic acid [2-fluoro-4-(2-oxo-2H-pyridin-1-yl)-phenyl]-amide;
1-[3-(4-Chloro-phenyl)-ureido]-cyclopropanecarboxylic acid [2-fluoro-4-(2-oxo-2H-pyridin-1-yl)-phenyl]-amide;
1-[3-(4-Chloro-phenyl)-ureido]-cyclopropanecarboxylic acid [2-fluoro-4-(5-methyl-pyrazol-1-yl)-phenyl]-amide;
(1R,2S)-1-[3-(4-Chloro-phenyl)-ureido]-2-hydroxymethyl-cyclopropanecarboxylic acid [2-fluoro-4-(5-methyl-pyrazol-1-yl)-phenyl)-amide;
(1S,2S)-1-[3-(4-Chloro-phenyl)-ureido]-2-hydroxymethyl-cyclopropanecarboxylic acid [2-fluoro-4-(5-methyl-pyrazol-1-yl)-phenyl)-amide;
2-[3-(4-Chloro-phenyl)-1-cyclopropylmethyl-ureido]-N-[2-fluoro-4-(5-methyl-pyrazol-1-yl)-phenyl]-acetamide;
1-[3-(4-Chloro-phenyl)-ureido]-cyclopropanecarboxylic acid [4-(3,5-dimethyl-pyrazol-1-yl)-2-fluoro-phenyl]-amide;
2-[3-(4-Chloro-phenyl)-1-cyclopropylmethyl-ureido]-N-[4-(3,5-dimethyl-pyrazol-1-yl)-2-fluoro-phenyl]-acetamide;
(1R,2S)-1-[3-(4-Chloro-phenyl)-ureido)-2-hydroxymethyl-cyclopropanecarboxylic acid (2-fluoro-4-pyrazol-1-yl-phenyl)-amide;
(1S,2S)-1-[3-(4-Chloro-phenyl)-ureido]-2-hydroxymethyl-cyclopropanecarboxylic acid (2-fluoro-4-pyrazol-1-yl-phenyl)-amide;
(1R,2S)-1-[3-(4-Chloro-phenyl)-ureido]-2-hydroxymethyl-cyclopropanecarboxylic acid [2-fluoro-4-(3-methyl-pyrazol-1-yl)-phenyl]-amide;
(1S,2S)-1-[3-(4-Chloro-phenyl)-ureido]-2-hydroxymethyl-cyclopropanecarboxylic acid [2-fluoro-4-(3-methyl-pyrazol-1-yl)-phenyl]-amide;
(1R,2S)-1-[3-(4-Chloro-phenyl)-ureido]-2-hydroxymethyl-cyclopropanecarboxylic acid [2-fluoro-4-(2-methyl-imidazol-1-yl)-phenyl]-amide;
(1S,2S)-1-[3-(4-Chloro-phenyl)-ureido]-2-hydroxymethyl-cyclopropanecarboxylic acid [2-fluoro-4-(2-methyl-imidazol-1-yl)-phenyl]-amide;
(1R,2S)-1-[3-(4-Chloro-phenyl)-ureido]-2-hydroxymethyl-cyclopropanecarboxylic acid [4-(2,5-dihydro-pyrrole-1-carbonyl)-2-fluoro-phenyl]-amide;
(1S,2S)-1-[3-(4-Chloro-phenyl)-ureido]-2-hydroxymethyl-cyclopropanecarboxylic acid [4-(2,5-dihydro-pyrrole-1-carbonyl)-2-fluoro-phenyl]-amide;
(1R,2S)-1-[3-(4-Chloro-phenyl)-ureido]-2-hydroxymethyl-cyclopropanecarboxylic acid [2-fluoro-4-(pyrrolidine-1-carbonyl)-phenyl]-amide;
(1S,2S)-1-[3-(4-Chloro-phenyl)-ureido]-2-hydroxymethyl-cyclopropanecarboxylic acid [2-fluoro-4-(pyrrolidine-1-carbonyl)-phenyl]-amide;
(1R,2S)-2-(Acetylamino-methyl)-1-[3-(4-chloro-phenyl)-ureido]-cyclopropanecarboxylic acid (3-fluoro-2′-methanesulfonyl-biphenyl-4-yl)-amide;
(1S,2S)-2-(Acetylamino-methyl)-1-[3-(4-chloro-phenyl)-ureido]-cyclopropanecarboxylic acid (3-fluoro-2′-methanesulfonyl-biphenyl-4-yl)-amide;
or a pharmaceutically acceptable salt thereof.

23. A process for the preparation of compounds of Formula I, wherein P¹ is a protecting group, Y¹ is a halogen and X¹, X², A, M, and Q are as defined above, comprising
(a) contacting an amino acid having Formula III with a reagent capable of forming a protecting group on the amino group of the amino acid to form a compound with Formula IV 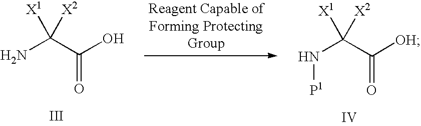
(b) activating of the carboxylic acid of Formula IV and contacting it with an amino compound of the formula H₂N-M-Y¹ to form a compound of Formula V 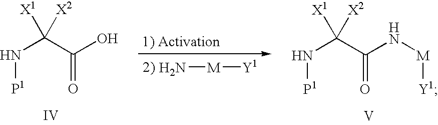
(c) coupling the compound of Formula V with a compound having Q to form a compound of Formula VI 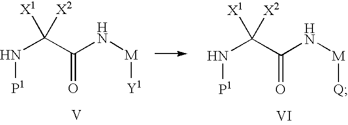
(d) removing the amino protecting group of the compound of Formula VI and contacting the resulting free amine with an isocyanate having A to form a compound of Formula I

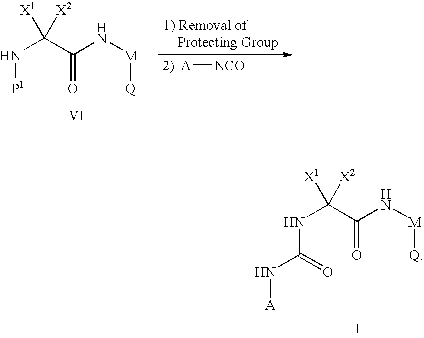

24. A process for the preparation of compounds of Formula I, wherein P¹ is a protecting group, and X¹, X², A, M, and Q are as defined above, comprising
(a) contacting an amino acid having Formula X with a reagent capable of forming a protecting group on the amino group of the amino acid to form a compound with Formula XI 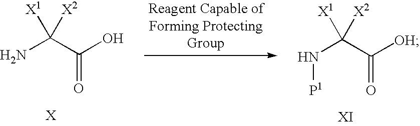
(b) activating of the carboxylic acid of Formula XI and contacting it with an amino compound of the formula H₂N-M-Q to form a compound of Formula XII
and 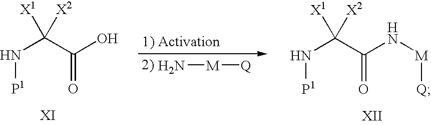
(c) removing the amino protecting group of the compound of Formula XII and contacting the resulting free amine with an isocyanate having A to form a compound of Formula I

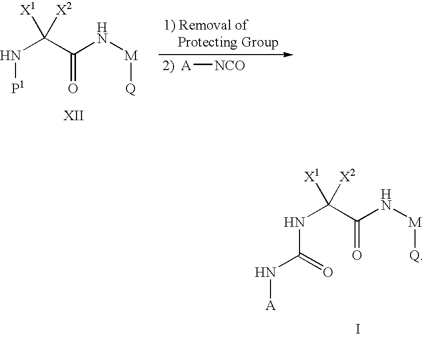

25. A process for the preparation of compounds of Formula I, wherein A, M, Q and R² are as defined above, comprising
(a) contacting a compound of Formula XVII with a bromoacetyl chloride of the Formula XX to form a compound of Formula XXI 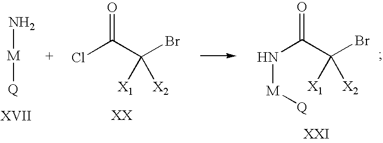
(b) contacting a compound of Formula XXI with an amine of Formula XXII to form a compound of Formula XXIII 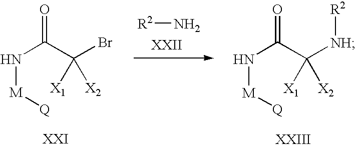
and
(c) contacting a compound of Formula XXIII with an isocyanate having A to form a compound of Formula I

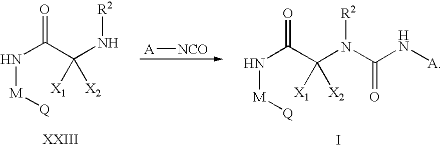

26. A process for the preparation of compounds of Formula I, wherein P¹ and P² are independent protecting groups and A, M, and Q are as defined above, comprising
(a) base catalyzed ring opening of a compound of Formula XXVIII to form a compound of Formula XXIX 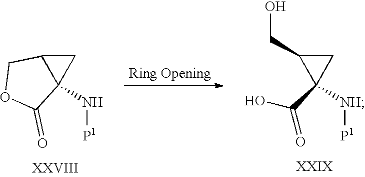
(b) contacting a compound of Formula XXIX with a reagent capable of forming a protecting group on the hydroxyl groups followed by contacting the resulting intermediate with a reagent capable of selective deprotection of the carboxylic acid hydroxyl group to form a compound with Formula XXX 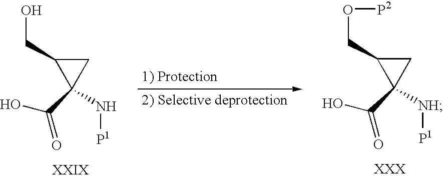
(c) activating the carboxylic acid of Formula XXX and contacting it with an amino compound of the formula XXXI to form a compound of Formula XXXII 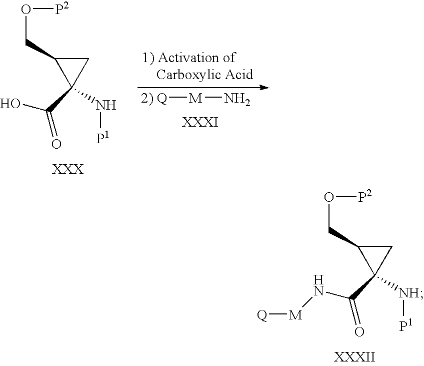
and
(d) removing the amino protecting group of the compound of Formula XXXII and contacting the resulting free amine with an isocyanate having A to from a compound of Formula I

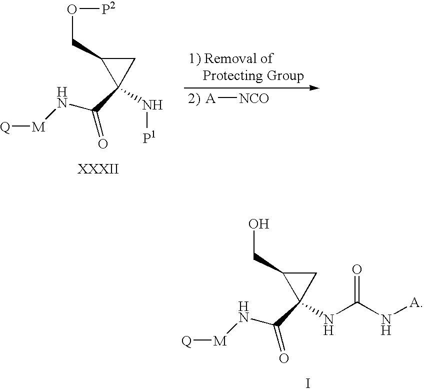

27. A process for the preparation of compounds of Formula I, wherein P¹ and P² are independent protecting groups and A, M, and Q are as defined above, comprising
(a) contacting a compound of Formula XXXIII with a reagent capable of selectively forming a protecting group on the alcohol hydroxyl group to form a compound with Formula XXXIV 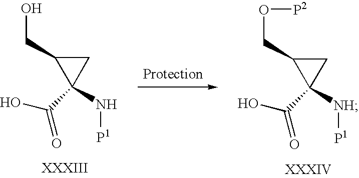
(b) activating the carboxylic acid of Formula XXXIV and contacting it with an amino compound of the formula XXXV to form a compound of Formula XXXVI 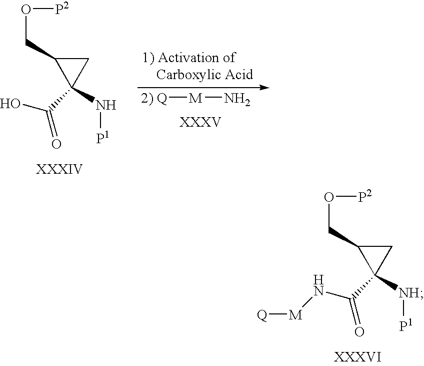
(c) removing the amino protecting group of the compound of Formula XXXVI and contacting the resulting free amine with an isocyanate having A to from a compound of Formula XXXVII 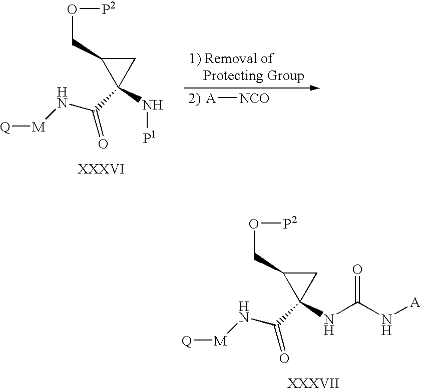
and
(d) removing the alcohol hydroxy protecting group of the compound of Formula XXXVII to from a compound of Formula I

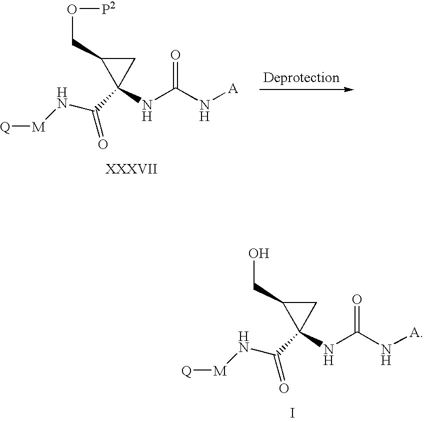

28. A process for the preparation of compounds of Formula I, wherein P¹ and P² are independently protecting groups and A, M, and Q are as defined above, comprising
(a) contacting a compound of Formula XXXVIII with acid to form a compound of Formula XXXIX 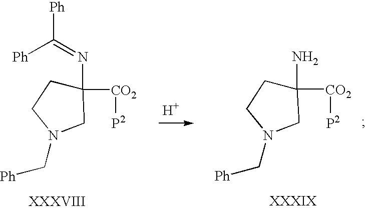
(b) contacting a compound of Formula XXXIX with a reagent capable of forming a protecting group on the amino moiety to form a compound of Formula XL 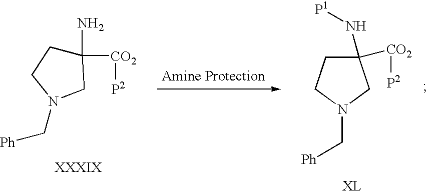
(c) contacting a compound of Formula XL with a reagent capable of forming a protecting group on the heterocycle nitrogen to form a compound of Formula XLI 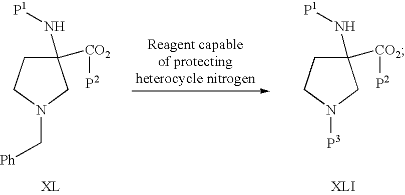
(d) contacting a compound of Formula XLI with a reagent capable of removing the protecting group of the carboxylic acid to form a compound of Formula XLII 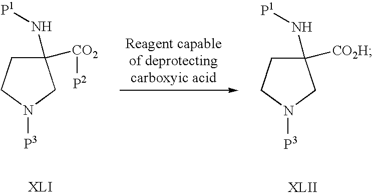
(e) activating the carboxylic acid of Formula XLII and contacting it with an amino compound of the formula XLIII to form a compound of Formula XLIV 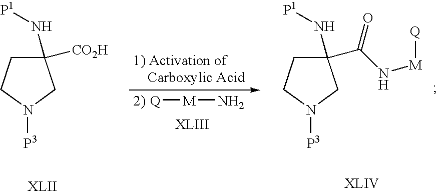
and
(f) removing the amino protecting group of the compound of Formula XLIV and contacting the resulting free amine with an isocyanate having A to from a compound of Formula I

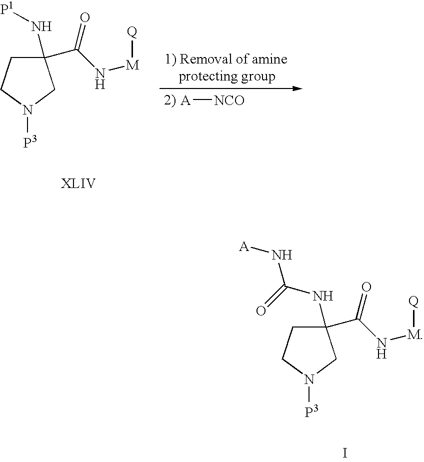

29. A method of treating or preventing thrombotic disorders in a mammal comprising administering to said mammal a therapeutically effective amount of a compound according to claims 1 or 22.

30. The method of claim 29, wherein said disorder is venous thrombosis.

31. The method of claim 29, wherein said disorder is arterial thrombosis.

32. The method of claim 29, wherein said disorder is pulmonary embolism.

33. The method of claim 29, wherein said disorder is myocardial infarction.

34. The method of claim 29, wherein said disorder is cerebral infarction.

35. The method of claim 29, wherein said disorder is restenosis.

36. The method of claim 29, wherein said disorder is cancer.

37. The method of claim 29, wherein said disorder is angina.

38. The method of claim 29, wherein said disorder is diabetes.

39. The method of claim 29, wherein said disorder is atrial fibrillation.

40. The method of claim 29, wherein said disorder is heart failure.

41. A method of inhibiting Factor Xa in a mammal comprising administering to said mammal a compound according to claims 1 or 22.l

42. A pharmaceutical formulation comprising a compound of claim 1 or 22 admixed with a carrier, diluent, or excipient.
